(19) United States
(12) Patent Application Publication
(10) Pub. No.: US 2024/0156980 A1
(43) Pub. Date: May 16, 2024

(54) PROTEASE CLEAVABLE PRODRUGS

(71) Applicant: MOLECULAR PARTNERS AG, Schlieren (CH)

(72) Inventors: Andreas BOSSHART, Schlieren (CH); Sebastian GRIMM, Schlieren (CH); Julia AHLSKOG, Schlieren (CH); Bernd SCHLERETH, Schlieren (CH)

(21) Appl. No.: 18/281,152

(22) PCT Filed: Mar. 9, 2022
(86) PCT No.: PCT/IB2022/052118

§ 371 Date: Sep. 8, 2023

Related U.S. Application Data
(60) Provisional application No. 63/158,785, filed on Mar. 9, 2021.
(60) Provisional application No. 63/159,043, filed on Mar. 10, 2021.

(51) Int. Cl. A61K 47/68; A61P 35/00

(52) U.S. Cl. CPC A61K 47/6889; A61K 47/6811; A61K 47/6843; A61K 47/6849; A61K 47/6851; A61P 35/00

(57) ABSTRACT

The application relates to prodrugs comprising a drug molecule connected by a protease-cleavable peptide linker to a binder, which reversibly inhibits a biological activity of the drug molecule, and to the inhibitory binders themselves. Also described are nucleic acids encoding the recombinant proteins described herein, and methods of making said recombinant proteins, as well as methods of treatment and medical uses of the recombinant proteins.

### CROSS-REFERENCE TO RELATED APPLICATIONS

The present application claims the benefit of priority to U.S. 63/158,785, filed on Mar. 9, 2021; and U.S. 63/159,043, filed on Mar. 10, 2021. The disclosures of these patent applications are incorporated herein for all purposes by reference in their entirety.

### FIELD OF THE INVENTION

This application relates to prodrugs comprising a drug molecule connected by a protease-cleavable peptide linker to a binder, which reversibly inhibits a biological activity of the drug molecule, and to the inhibitory binders themselves. Also described are nucleic acids encoding the recombinant proteins described herein, and methods of making said recombinant proteins, as well as methods of treatment and medical uses of the recombinant proteins.

### BACKGROUND OF THE INVENTION

Drug candidates must meet certain efficacy standards, but they must also exhibit an acceptable safety profile. Many drug molecules have some adverse off-target and/or on-target side effects, and some drug molecules may also cause adverse on-target effects due to exaggerated and adverse pharmacologic effects at the intended target of the drug molecule. For certain drug classes adverse effects cannot be avoided so they must be mitigated. There are several options for this. For example, nonsteroidal anti-inflammatory drugs (NSAIDs) can damage the lining of the stomach, so they are often co-administered with an agent to protect the stomach, such as the proton-pump inhibitor omeprazole. For other drug molecules, the adverse effects, or the risk thereof, can be mitigated using a particular dosage regime such that the drug is administered to the patient in multiple doses or continuously over a longer period of time. Examples of this may include taking a drug multiple times a day, or intravenous (IV) infusion of a drug.

While divided doses and IV drug infusions are recognised and acceptable ways of dosing medication, they are not without drawbacks. Dosage regimes that are onerous on the patient, such as those requiring medication multiple times over a relatively short time period, are associated with poor patient compliance and consequently worse treatment outcomes. While IV infusion methods are less likely to suffer from poor patient compliance, the patient must be under medical care. This is disruptive for patients and results in added strain on the healthcare system. Consequently, there is a need in the art for improved ways of mitigating adverse drug effects.

One class of drugs typically associated with adverse effects are anti-cancer agents. T-cell engager drugs (TCEs) direct cytotoxic T-cell responses towards tumor cells by binding simultaneously to a tumor-associated antigen (TAA) on target cells and to CD3 receptors on T-cells, thereby forming an artificial immune synapse. They have been shown to be very potent anti-tumor drugs, as exemplified by blinatumomab, an α-CD19×α-CD3 bispecific antibody. However, the development of TCEs for hematological and solid tumors has been hampered by several factors. As well as their anti-tumor activity, TCEs are also associated with systemic endothelial activation and massive lymphocyte redistribution, as well as neurological toxicities, particularly following first dose administration (Velasquez, Blood, 2018, 131(1), 30-38). TCEs are also associated with severe toxicity elicited by on-target/off-tumor recruitment of T-cells and cytokine release syndrome (CRS) and hypercytokinemia, also known as a “cytokine storm”.

CRS or hypercytokinemia typically occurs rapidly after the first dose of a drug and is characterised by an uncontrolled and excessive release of cytokines in the body. While cytokine release is a critical part of normal immune function, release of too many cytokines into the blood too quickly can cause symptoms such as high fever, inflammation, severe fatigue, nausea, and sometimes even multiple organ failure and death. A clinical trial for the drug Theralizumab, intended for the treatment of B cell chronic lymphocytic leukaemia and rheumatoid arthritis, had to be abandoned after the participants developed severe hypercytokinemia. The onset of symptoms occurred within an hour of dosing, and all of the participants in the trial required urgent hospital care. CRS is also caused by a large, rapid release of cytokines into the blood from immune cells affected by the immunotherapy. Symptoms of CRS include fever, nausea, headache, rash, rapid heartbeat, low blood pressure, and trouble breathing. Sometimes, CRS may be severe or life threatening.

Severe adverse effects of immunotherapies due to on-target/off-tumor toxicity arise in patients who have target antigen expressed on both tumor and healthy tissue. Such an expression pattern is typical for many target antigens used in targeted cancer therapies, such as, e.g., certain members of the epidermal growth factor receptor (EGFR) family. An example of such an antigen is Her2, which is an attractive target for cancer therapy since it can be overexpressed 40- to 100-fold in tumors. Her2 has long been targeted therapeutically using monoclonal antibodies such as trastuzumab (Herceptin®). Her2 has also been targeted by immunotherapy. A Her2 CAR-T cell therapy based on the trastuzumab sequence was used to treat a patient with colorectal cancer, and, unfortunately, off-tumor targeting of the patient's cardiopulmonary system caused lethal toxicity (Morgan et al., Mol Ther., 2010; 18(4):843-851). Serum samples after cell infusion showed marked increases of various cytokines, consistent with a cytokine storm, and this cytokine storm was likely triggered by the recognition of low levels of Her2 on lung epithelial cells by the administered cells. This adverse effect was not foreseen based on clinical studies of trastuzumab or based on preclinical animal studies.

These toxicities often impact clinical trial design and dose escalation strategies, and have proven dose limiting due to severity, especially in patients with high disease burden. Pre-medication and/or active intervention may also be required, ultimately leading to complex clinical trial design. Several strategies have been devised for clinical management of CRS associated with the administration of T-cell engager drugs. These include step-dosing (stepwise dose-escalation), pre-treatment with steroids (especially dexamethasone) or treatment with tocilizumab (anti-IL6 receptor antibody) (see e.g., Aldoss et al., Current Oncology Reports. 2019, 21:4). Pre-treatment with steroids delays the onset of treatment, which is not recommended for aggressive disease states, and use of steroids may be contraindicated in patients with high body mass index (BMI) and/or blood pressure. Treatment with tocilizumab to avoid CRS was approved by the FDA in 2017. However, the immunosuppressive effect of this drug can leave patients vulnerable to other infectious diseases.

Taken together, there remains a need for novel or improved approaches to avoiding, reducing or mitigating the adverse effects, or the risk thereof, of drugs used for the treatment of diseases, including cancer.

### SUMMARY OF THE INVENTION

This application seeks to provide a novel approach to avoiding or mitigating adverse effects, or the risk thereof, following administration of a drug molecule. The present invention provides a method of inhibiting the biological activity of a drug molecule by a binding moiety which reversibly binds to the drug molecule and which is connected to the drug molecule by a protease-cleavable peptide linker. The biological activity of the drug molecule that is inhibited by the binding moiety may be, for example, the binding of the drug molecule to a biological target. Upon cleavage of the peptide linker by a protease, such as a protease expressed in tumor tissue, the binding moiety dissociates from the drug molecule, thereby releasing active drug molecule into the body at the site of proteolytic cleavage. This method avoids a peak in active drug molecule concentration in the body shortly after administration and localizes the release of active drug molecule to sites of expression of an appropriate protease. An example of a beneficial application of this approach is the reduction of the risk of on-target/off-tumor toxicity and CRS following administration of a prodrug TCE protein, comprising a TCE and an inhibitory binding moiety connected to it by a protease-cleavable peptide linker.

The application describes novel prodrug proteins comprising (i) a binding moiety and (ii) a drug molecule; wherein said binding moiety reversibly binds to said drug molecule; wherein said binding moiety, when bound, inhibits a biological activity of said drug molecule; wherein said binding moiety and said drug molecule are connected by a peptide inker; and wherein said peptide linker comprises one or more protease cleavage site(s). Preferentially, protease(s) that can cleave the peptide linker are expressed at elevated levels in the target tissue of the drug molecule, such as a tumor tissue. Further described in this application are specific binding moieties which can be used in such a prodrug approach in combination with various drug molecules. The binding moieties of the invention may be anti-idiotypic binders of the drug molecule. In some embodiments, prodrug proteins of the invention further comprise a serum half-life extending moiety. Preferentially, such half-life extending moiety is covalently connected to the inhibitory binding moiety, such that the half-life extending moiety is cleaved off the drug molecule together with the binding moiety upon proteolytic cleavage of the peptide linker. In this way, the prodrug protein has an extended serum half-life, but the active drug molecule, released upon proteolytic cleavage of the peptide linker, does not. Biological activity of the drug molecule is therefore relatively short in duration, thereby further contributing to the avoidance of on-target/off-tumor toxicity that could occur upon distribution of active drug molecule from the tumor tissue to other sites in the body.

In a specific application of this prodrug approach, the present invention provides a DARPin® TCE prodrug (CD3-PDD) comprising a TAA-binder and a CD3-binder, linked via a protease-cleavable linker to an anti-idiotypic anti-CD3-binder binding moiety (termed Binder hereafter), see FIG. 1. This α-TAA×α-CD3×Binder prodrug is unable to bind and recruit T-cells in its non-cleaved state, but is designed to become activated in the tumor microenvironment (TME) upon cleavage of the linker by tumor-associated proteases.

The underlying idea of such a protease-activatable prodrug is to exploit the miss-regulation of proteases in the tumor-microenvironment, namely by constructing a prodrug molecule that is non-active in circulation and healthy tissue, but becomes activated once it is in the TME by cleavage of the protease-susceptible linker by tumor-associated proteases, see FIG. 1.

The blocking concept of the prodrug relies on the phenomenon of “forced proximity”, i.e. the very high concentration of the Binder in proximity of the CD3-binder. This is due to the distance constraints imposed by the linker, allowing the Binder to only access a certain volume around the CD3-binder. Upon cleavage of the linker by tumor-associated proteases, the forced proximity is abolished, and the Binder can diffuse away freely, restricted only by the off-rate between Binder and CD3-binder.

In one embodiment, a half-life extending moiety (such as a HSA-binder) is attached to the blocking moiety (i.e. the Binder) of the TCE prodrug molecule. This provides another layer of safety: upon cleavage, the T-cell engager is rendered active, but at the same time loses its half-life extending moiety. Therefore, an active TCE that leaks back from the TME into circulation is cleared quickly from the system due to its small size and its short half-life.

In summary, a conditionally activatable TCE prodrug is described herein, which shows similar efficacy, but none of the toxicity, of the corresponding constitutively active (i.e. non-blocked) TCE. The described prodrug approach holds promise for the development of future prodrug TCE therapeutics, enabling the utilization of tumor-associated antigens (TAAs), even if they are also expressed in some non-targeted tissue(s) (i.e. healthy tissue(s)), as targets for highly potent TCEs.

Taken together, the present invention provides conditionally activatable prodrugs comprising a drug molecule connected by protease-cleavable peptide linker to a binding moiety, which, when bound, inhibits a biological activity of the drug molecule. Furthermore, the present invention provides inhibitory binding moieties as such, which can be used in various prodrugs. Also provided are nucleic acids encoding the recombinant proteins described herein, and methods of making said recombinant proteins using host cells, as well as medical uses and methods of treatment using the recombinant proteins.

Based on the disclosure provided herein, those skilled in the art will recognize, or be able to ascertain using no more than routine experimentation, many equivalents to the specific embodiments of the invention described herein. Such equivalents are intended to be encompassed by the following embodiments (E).

In a first embodiment, the invention relates to a recombinant protein comprising (i) a binding moiety and (ii) a drug molecule;
wherein said binding moiety reversibly binds to said drug molecule;
wherein said binding moiety, when bound, inhibits a biological activity of said drug molecule; wherein said binding moiety and said drug molecule are connected by a peptide linker; and wherein said peptide linker comprises a protease cleavage site.

In a second embodiment, the invention relates to the recombinant protein of embodiment 1, wherein said binding moiety comprises an antibody, an alternative scaffold, or a polypeptide.

In a third embodiment, the invention relates to the recombinant protein of embodiments 1 or 2, wherein said binding moiety comprises an immunoglobulin molecule or a fragment thereof.

In a fourth embodiment, the invention relates to the recombinant protein of embodiments 1 or 2, wherein said binding moiety comprises a non-immunoglobulin molecule.

In a fifth embodiment, the invention relates to the recombinant protein of any of embodiments 1 to 4, wherein said binding moiety comprises an antigen binding domain that is derived from a monoclonal antibody, a polyclonal antibody, a recombinant antibody, a chimeric antibody, a human antibody, a humanized antibody, a single-domain antibody, a heavy chain variable domain (VH), a light chain variable domain (VL), or a variable domain (VHH).

In a sixth embodiment, the invention relates to the recombinant protein of any of embodiments 1 to 4, wherein said binding moiety comprises an antigen binding domain that is derived from or is related to an adnectin, a monobody, an affibody, an affilin, an affimer, an aptamer, an affitin, an alphabody, an anticalin, a repeat protein domain, an armadillo repeat domain, an atrimer, an avimer, an ankyrin repeat domain, a fynomer, a knottin, a Kunitz domain, or a T cell receptor (TCR).

In a seventh embodiment, the invention relates to the recombinant protein of any preceding embodiment, wherein said biological activity of said drug molecule is binding of said drug molecule to a biological target.

In an eighth embodiment, the invention relates to the recombinant protein of any preceding embodiment, wherein said biological activity of said drug molecule is an enzymatic activity.

In a ninth embodiment, the invention relates to the recombinant protein of any preceding embodiment, wherein cleavage of said peptide linker at said protease cleavage site upon administration of said recombinant protein to a mammal allows release of said drug molecule from the said binding moiety.

In a tenth embodiment, the invention relates to the recombinant protein of embodiment 9, wherein said mammal is a human.

In an eleventh embodiment, the invention relates to the recombinant protein of any of embodiments 9 and 10, wherein said cleavage of said peptide inker occurs in tumor tissue.

In a twelfth embodiment, the invention relates to the recombinant protein of any preceding embodiment, wherein said protease cleavage site is a site recognized by a protease present in tumor tissue.

In a thirteenth embodiment, the invention relates to the recombinant protein of any preceding embodiment, wherein said binding moiety binds said drug molecule with a dissociation constant (K_{D}) of less than about 1 μM, such as less than about 1 μM, less than about 500 nM, less than about 250 nM, less than about 100 nM or less than about 50 nM.

In a fourteenth embodiment, the invention relates to the recombinant protein of any preceding embodiment, wherein said binding moiety binds said drug molecule with a dissociation constant (K_{D}) of between about 1 μM and about 10 μM, such as of between about 1 μM and about 10 μM, of between about 1 μM and about 20 μM, of between about 1 μM and about 50 μM, or of between about 1 μM and about 100 μM.

In a fifteenth embodiment, the invention relates to the recombinant protein of embodiments 13 or 14, wherein said dissociation constant (K_{D}) is measured in phosphate buffered saline (PBS).

In a sixteenth embodiment, the invention relates to the recombinant protein of any preceding embodiment, wherein said binding moiety comprises a designed ankyrin repeat domain.

In a seventeenth embodiment, the invention relates to the recombinant protein of embodiment 16, wherein said designed ankyrin repeat domain comprises an ankyrin repeat module comprising an amino acid sequence selected from the group consisting of (1) SEQ ID NOs: 45 to 64 and (2) sequences in which up to 9 amino acids in any of SEQ ID NOs: 45 to 64 are substituted by other amino acids.

In an eighteenth embodiment, the invention relates to the recombinant protein of embodiments 16 or 17, wherein said designed ankyrin repeat domain comprises an amino acid sequence selected from the group consisting of (1) SEQ ID NOs: 1 to 12 and (2) sequences that have at least 85% amino acid sequence identity with any of SEQ ID NOs: 1 to 12.

In a nineteenth embodiment, the invention relates to the recombinant protein of any preceding embodiment, wherein said drug molecule comprises an antibody, an alternative scaffold, or a polypeptide.

In a twentieth embodiment, the invention relates to the recombinant protein of any preceding embodiment, wherein said drug molecule comprises an immunoglobulin molecule or a fragment thereof.

In a twenty-first embodiment, the invention relates to the recombinant protein of any preceding embodiment, wherein said drug molecule comprises a non-immunoglobulin molecule.

In a twenty-second embodiment, the invention relates to the recombinant protein of any preceding embodiment, wherein said drug molecule comprises an antigen binding domain that is derived from a monoclonal antibody, a polyclonal antibody, a recombinant antibody, a chimeric antibody, a human antibody, a humanized antibody, a single-domain antibody, a heavy chain variable domain (VH), a light chain variable domain (VL), or a variable domain (VHH).

In a twenty-third embodiment, the invention relates to the recombinant protein of any preceding embodiment, wherein said drug molecule comprises an antigen binding domain that is derived from or is related to an adnectin, a monobody, an affibody, an affilin, an affimer, an aptamer, an affitin, an alphabody, an anticalin, a repeat protein domain, an armadillo repeat domain, an atrimer, an avimer, an ankyrin repeat domain, a fynomer, a knottin, a Kunitz domain, or a T cell receptor (TCR).

In a twenty-fourth embodiment, the invention relates to the recombinant protein of any preceding embodiment, wherein said drug molecule has binding specificity for CD3.

In a twenty-fifth embodiment, the invention relates to the recombinant protein of any of any preceding embodiment, wherein said drug molecule comprises at least one binding domain with binding specificity for a tumor-associated antigen (TAA).

In a twenty-sixth embodiment, the invention relates to the recombinant protein of any preceding embodiment, wherein said drug molecule comprises a designed ankyrin repeat domain.

In a twenty-seventh embodiment, the invention relates to the recombinant protein of embodiment 26, wherein said designed ankyrin repeat domain has binding specificity for CD3.

In a twenty-eighth embodiment, the invention relates to the recombinant protein of any of embodiments 26 and 27, wherein said designed ankyrin repeat domain comprises an amino acid sequence selected from the group consisting of (1) SEQ ID NOs: 13 to 17 and (2) sequences that have at least 85% amino acid sequence identity with any of SEQ ID NOs: 13 to 17.

In a twenty-ninth embodiment, the invention relates to the recombinant protein of any of embodiments 27 and 28, wherein said designed ankyrin repeat domain binds to CD3 with a dissociation constant (K_{D}) of less than about 100 nM.

In a thirtieth embodiment, the invention relates to the recombinant protein of any of embodiments 1 to 25, wherein said drug molecule comprises an antibody.

In a thirty-first embodiment, the invention relates to the recombinant protein of embodiment 30, wherein said antibody has binding specificity for CD3.

In a thirty-second embodiment, the invention relates to the recombinant protein of any preceding embodiment, wherein said drug molecule is a T-cell engager drug molecule (TCE).

In a thirty-third embodiment, the invention relates to the recombinant protein of embodiment 32, wherein said TCE comprises a binding domain which binds to CD3 and further comprises a binding domain which binds a tumor-associated antigen (TAA).

In a thirty-fourth embodiment, the invention relates to the recombinant protein of any of embodiments 32 and 33, wherein binding of said binding moiety to said TCE drug molecule inhibits binding of said TCE drug molecule to T cells and/or activation of T cells.

In a thirty-fifth embodiment, the invention relates to the recombinant protein of any one of embodiments 32 to 34, wherein said TCE is a bispecific or multispecific antibody.

In a thirty-sixth embodiment, the invention relates to the recombinant protein of any one of embodiments 32 to 34, wherein said TCE is a bispecific or multispecific ankyrin repeat protein.

In a thirty-seventh embodiment, the invention relates to the recombinant protein of any one of embodiments 33 to 36, wherein said binding domain which binds to CD3 is located on the C-terminal side of said binding domain which binds a tumor-associated antigen (TAA).

In a thirty-eighth embodiment, the invention relates to the recombinant protein of any preceding embodiment, wherein said binding moiety is an anti-idiotypic binder of said drug molecule.

In a thirty-ninth embodiment, the invention relates to the recombinant protein of embodiment 38, wherein said binding moiety is an anti-idiotypic binder of said designed ankyrin repeat domain having binding specificity for CD3.

In a fortieth embodiment, the invention relates to the recombinant protein of embodiment 38, wherein said binding moiety is an anti-idiotypic binder of said antibody having binding specificity for CD3.

In a forty-first embodiment, the invention relates to the recombinant protein of any preceding embodiment, wherein said binding moiety, said drug molecule and said peptide linker are arranged, from N-terminus to C-terminus, in the following format: drug molecule—peptide linker—binding moiety.

In a forty-second embodiment, the invention relates to the recombinant protein of any of embodiments 1 to 41, wherein said binding moiety, said binding domain which binds to CD3, said binding domain which binds a tumor-associated antigen (TAA), and said peptide linker are arranged, from N-terminus to C-terminus, in the following format: binding domain which binds a tumor-associated antigen (TAA)—binding domain which binds to CD3—peptide inker—binding moiety.

In a forty-third embodiment, the invention relates to the recombinant protein of any preceding embodiment, further comprising an agent which extends the serum half-life of the recombinant protein in a mammal.

In a forty-fourth embodiment, the invention relates to the recombinant protein of embodiment 43, wherein said agent which extends the serum half-life of the recombinant protein in a mammal has binding specificity for serum albumin.

In a forty-fifth embodiment, the invention relates to the recombinant protein of embodiment 44, wherein said agent which extends the serum half-life of the recombinant protein in a mammal comprises a designed ankyrin repeat domain with binding specificity for serum albumin.

In a forty-sixth embodiment, the invention relates to the recombinant protein of embodiment 45, wherein said designed ankyrin repeat domain with binding specificity for serum albumin comprises an amino acid sequence selected from the group consisting of (1) SEQ ID NOs: 65 to 67 and (2) sequences that have at least 85% amino acid sequence identity with any of SEQ ID NOs: 65 to 67. In a further embodiment, the invention relates to the recombinant protein of embodiment 46, wherein said designed ankyrin repeat domain binds to human serum albumin with a dissociation constant (K_{D}) of less than about 100 nM.

In a forty-seventh embodiment, the invention relates to the recombinant protein of any of embodiments 43 to 46, wherein said agent which extends the serum half-life of the recombinant protein in a mammal is located at the same side of said peptide linker as said binding moiety.

In a forty-eighth embodiment, the invention relates to the recombinant protein of embodiment 47, wherein said binding moiety and said agent which extends the serum half-life of the recombinant protein in a mammal are both located at the C-terminal side of said peptide linker.

In a forty-ninth embodiment, the invention relates to the recombinant protein of any of embodiments 43 to 47, wherein said agent which extends the serum half-life of the recombinant protein in a mammal is located at the C-terminal side of said binding moiety.

In a fiftieth embodiment, the invention relates to the recombinant protein of any of embodiments 43 to 49, wherein said binding moiety, said binding domain which binds to CD3, said binding domain which binds a tumor-associated antigen (TAA), said peptide linker, and said agent which extends the serum half-life of the recombinant protein in a mammal are arranged, from N-terminus to C-terminus, in the following format: binding domain which binds a tumor-associated antigen (TAA)—binding domain which binds to CD3—peptide linker—binding moiety—agent which extends the serum half-life of the recombinant protein in a mammal.

In a fifty-first embodiment, the invention relates to a nucleic acid encoding the recombinant protein of any of the preceding embodiments.

In a fifty-second embodiment, the invention relates to a host cell comprising the nucleic acid molecule of embodiment 51.

In a fifty-third embodiment, the invention relates to a method of making the recombinant protein of any one of embodiments 1 to 50, comprising culturing the host cell of embodiment 52 under conditions wherein said recombinant protein is expressed.

In a fifty-fourth embodiment, the invention relates to the method of embodiment 53, wherein said host cell is a prokaryotic host cell.

In a fifty-fifth embodiment, the invention relates to the method of embodiment 53, wherein said host cell is a eukaryotic host cell.

In a fifty-sixth embodiment, the invention relates to a pharmaceutical composition comprising the recombinant protein of any one of embodiments 1 to 50 or the nucleic acid of embodiment 51 and additionally comprising a pharmaceutically acceptable carrier or excipient.

In a fifty-seventh embodiment, the invention relates to the recombinant protein of any one of embodiments 1 to 50, the nucleic acid of embodiment 51 or the pharmaceutical composition of embodiment 56 for use in therapy.

In a fifty-eighth embodiment, the invention relates to the recombinant protein, nucleic acid or pharmaceutical composition for use according to embodiment 57, for use in treating a proliferative disease, optionally wherein said proliferative disease is cancer.

In a fifty-ninth embodiment, the invention relates to a method of treatment comprising the step of administering to a subject in need thereof the recombinant protein of any one of embodiments 1 to 50, the nucleic acid of embodiment 51 or the pharmaceutical composition of embodiment 56.

In a sixtieth embodiment, the invention relates to the method of embodiment 59, wherein said method is a method of treating a proliferative disease, optionally wherein said proliferative disease is cancer.

In a sixty-first embodiment, the invention relates to a method of T cell activation in a subject in need thereof, the method comprising the step of administering to said subject the recombinant protein of any one of embodiments 1 to 50, the nucleic acid of embodiment 51 or the pharmaceutical composition of embodiment 56.

In a sixty-second embodiment, the invention relates to a method of controlling release of an active drug molecule in vivo comprising administering the recombinant protein of any one of embodiments 1 to 50, the nucleic acid of embodiment 51 or the pharmaceutical composition of embodiment 56 to a subject in need thereof.

In a sixty-third embodiment, the invention relates to the method of any one of embodiments 59 to 62, wherein said subject is a human.

In a sixty-fourth embodiment, the invention relates to a method of controlling the biological activity of a drug molecule, the method comprising connecting a binding moiety as defined in any one of embodiments 1 to 6, 13 to 18 and 38 to 40 with a drug molecule as defined in any one of embodiments 19 to 37 with a peptide linker comprising a protease cleavage site to form a recombinant protein and administering said recombinant protein to a patient in need thereof, wherein said protease cleavage site is recognized by a protease present in tumor tissue.

In a sixty-fifth embodiment, the invention relates to the method of embodiment 64, wherein said biological activity of said drug molecule is binding of said drug molecule to a biological target.

In a sixty-sixth embodiment, the invention relates to the method of embodiment 64, wherein said biological activity of said drug molecule is an enzymatic activity.

In a sixty-seventh embodiment, the invention relates to a binding moiety having binding specificity for a drug molecule, wherein said binding moiety, when connected to said drug molecule by a peptide linker, inhibits a biological activity of said drug molecule.

In a sixty-eighth embodiment, the invention relates to the binding moiety of embodiment 67, wherein binding of said binding moiety to said drug molecule forms a complex that reversibly inhibits a biological activity of said drug molecule.

In a sixty-ninth embodiment, the invention relates to the binding moiety of any of embodiments 67 or 68, wherein said binding moiety is an anti-idiotypic binder of said drug molecule.

In a seventieth embodiment, the invention relates to the binding moiety of any of embodiments 67 to 69, wherein said biological activity of said drug molecule is binding of said drug molecule to a biological target.

In a seventy-first embodiment, the invention relates to the binding moiety of any of embodiments 67 to 69, wherein said biological activity of said drug molecule is an enzymatic activity.

In a seventy-second embodiment, the invention relates to the binding moiety of any one of embodiments 67 to 71, having a binding affinity (K_{D}) to said drug molecule of less than about 1 μM, such as less than about 1 μM, less than about 500 nM, less than about 250 nM, less than about 100 nM or less than about 50 nM.

In a seventy-third embodiment, the invention relates to the binding moiety of any one of embodiments 67 to 72, wherein said binding moiety binds said drug molecule with a dissociation constant (K_{D}) of between about 1 μM and about 10 μM, such as of between about 1 μM and about 10 μM, of between about 1 μM and about 20 μM, of between about 1 μM and about 50 μM, or of between about 1 μM and about 100 μM.

In a seventy-fourth embodiment, the invention relates to the binding moiety of embodiments 72 or 73, wherein said dissociation constant (K_{D}) is measured in phosphate buffered saline (PBS).

In a seventy-fifth embodiment, the invention relates to the binding moiety of any one of embodiments 67 to 74, wherein said binding moiety comprises an antibody, an alternative scaffold, or a polypeptide.

In a seventy-sixth embodiment, the invention relates to the binding moiety of any one of embodiments 67 to 75, wherein said binding moiety comprises an immunoglobulin molecule or a fragment thereof.

In a seventy-seventh embodiment, the invention relates to the binding moiety of any one of embodiments 67 to 76, wherein said binding moiety comprises a non-immunoglobulin molecule.

In a seventy-eighth embodiment, the invention relates to the binding moiety of any one of embodiments 67 to 77, wherein said binding moiety comprises an antigen binding domain that is derived from a monoclonal antibody, a polyclonal antibody, a recombinant antibody, a chimeric antibody, a human antibody, a humanized antibody, a single-domain antibody, a heavy chain variable domain (VH), a light chain variable domain (VL), or a variable domain (VHH).

In a seventy-ninth embodiment, the invention relates to the binding moiety of any one of embodiments 67 to 78, wherein said binding moiety comprises an antigen binding domain that is derived from or is related to an adnectin, a monobody, an affibody, an affilin, an affimer, an aptamer, an affitin, an alphabody, an anticalin, a repeat protein domain, an armadillo repeat domain, an atrimer, an avimer, an ankyrin repeat domain, a fynomer, a knottin, a Kunitz domain, or a T cell receptor (TCR).

In an eightieth embodiment, the invention relates to the binding moiety of any one of embodiments 67 to 79, wherein said binding moiety comprises a designed ankyrin repeat domain.

In an eighty-first embodiment, the invention relates to the binding moiety of embodiment 80, wherein said designed ankyrin repeat domain comprises an ankyrin repeat module comprising an amino acid sequence selected from the group consisting of (1) SEQ ID NOs: 45 to 64 and (2) sequences in which up to 9 amino acids in any of SEQ ID NOs: 45 to 64 are substituted by other amino acids.

In an eighty-second embodiment, the invention relates to the binding moiety of any of embodiments 80 or 81, wherein said designed ankyrin repeat domain comprises an amino acid sequence selected from the group consisting of (1) SEQ ID NOs: 1 to 12 and (2) sequences that have at least 85% amino acid sequence identity with any of SEQ ID NOs: 1 to 12.

In an eighty-third embodiment, the invention relates to a nucleic acid encoding the binding moiety of any of embodiments 67 to 82.

In an eighty-fourth embodiment, the invention relates to a nucleic acid encoding the designed ankyrin repeat domain of any of embodiments 80 to 82.

In an eighty-fifth embodiment, the invention relates to a host cell comprising the nucleic acid molecule of embodiments 83 or 84.

In an eighty-sixth embodiment, the invention relates to a method of making the binding moiety according to any one of embodiments 67 to 82, comprising culturing the host cell of embodiment 85 under conditions wherein said binding moiety is expressed.

In an eighty-seventh embodiment, the invention relates to the method of embodiment 86, wherein said host cell is a prokaryotic host cell.

In an eighty-eighth embodiment, the invention relates to the method of embodiment 86, wherein said host cell is a eukaryotic host cell.

In an eighty-ninth embodiment, the invention relates to a pharmaceutical composition comprising the binding moiety of any one of embodiments 67 to 82 or the nucleic acid of any one of embodiments 83 and 84 and additionally comprising a pharmaceutically acceptable carrier or excipient.

In a ninetieth embodiment, the invention relates to the binding moiety of any one of embodiments 67 to 82, the nucleic acid of any one of embodiments 83 and 84 or the pharmaceutical composition of embodiment 89 for use in therapy.

In a ninety-first embodiment, the invention relates to a method of treatment comprising the step of administering to a subject in need thereof the binding moiety of any one of embodiments 67 to 82, the nucleic acid of any one of embodiments 83 and 84 or the pharmaceutical composition of embodiment 89.

### BRIEF DESCRIPTION OF THE FIGURES

FIG. 1: Graphical illustration of the conditionally activatable prodrug approach. The prodrug molecule comprises (1) a drug molecule (in the illustrated example comprising a tumor associated antigen (TAA)-binding domain (α-TAA) and a CD3-binding domain (α-CD3)), (2) a binding moiety which can reversibly bind to the drug molecule and, when bound, inhibit a biological activity of the drug molecule (in the illustrated example an anti-Idiotypic binding domain against the paratope of α-CD3 (Binder)), and (3) optionally a half-fife extending moiety (in the illustrated example an HSA-binding domain (α-HSA) for half-life extension of the intact prodrug molecule). The linker between the drug molecule and the binding moiety (in the illustrated example the linker between α-CD3 and Binder) comprises a peptide linker that is cleavable by a protease (in the illustrated example by one or more proteases present in the tumor microenvironment (TME)). The prodrug molecule (in the illustrated example a DARPin® TCE prodrug (CD3-PDD)) is inactive upon injection into circulation, as the binding of the drug molecule to its target (in the illustrated example the binding to T-cells via α-CD3) is inhibited by the covalently linked binding moiety (in the illustrated example the Binder). Once reaching the target tissue of the drug molecule (in the illustrated example the tumor tissue), the peptide linker between the drug molecule and the binding moiety (in the illustrated example the peptide linker between α-CD3 and Binder) is cut by a protease present in the target tissue (in the illustrated example one or more tumor-associated proteases). Once the peptide linker is cleaved, the binding moiety diffuses away from the drug molecule and the drug molecule can exert its biological activity (in the illustrated example binding to TAA on tumor cells via its α-TAA arm and binding to CD3 on T-cells via its α-CD3 arm, leading to T-cell mediated tumor cell killing).

FIG. 2: Affinities (K_{D} in nM) of four different binding moieties (Binder #1 to Binder #4; SEQ ID NOs: 1 to 4, see Example 1) against five different CD3-specific binding domains (SEQ ID NOs: 13 to 17) (on top), determined by surface plasmon resonance (SPR) at room temperature. K_{D} values in nM were determined by using biotinylated Binders as ligands and CD3-specific binding domains as analytes. Parental Binder #1 (SEQ ID NO: 1) showed the highest affinity towards the five different CD3-binding domains, followed by Binder #4 (SEQ ID NO: 4) and the two low affinity binding moieties Binder #2 (SEQ ID NO: 2) and Binder #3 (SEQ ID NO: 3).

FIGS. 3A and B: Standard tumor cell killing assay using HCT 116 tumor cells and pan T-cells from one representative donor (out of three donors) and comparing active TCE constructs with two different anti-CD3 binding domains (C7v119 (SEQ ID NO: 15) with lower affinity for CD3 or C7v122 (SEQ ID NO: 16) with higher affinity for CD3) to their corresponding, non-cleavable DARPin® CD3-Prodrug (CD3-PDD NCL) counter-parts (i.e. containing a non-cleavable peptide linker instead of a protease-cleavable peptide linker). A) T-cell mediated killing of HCT 116 tumor cells by pan T-cells in the context of the CD3-binding domain C7v119 using either active TCE (empty square), CD3-PDD NCL containing Binder #3 (SEQ ID NO: 3) (empty triangle, down) or CD3-PDD NCL containing Binder #4 (SEQ ID NO: 4) (empty triangle, up) linked via non-cleavable linkers to the CD3-binding domain. Binder #4 with higher affinity towards the CD3-binding domain (see FIG. 2) shows a higher masking efficiency than lower affinity Binder #3. B) Same experimental setup as for A), but in the context of the CD3-binding domain C7v122 using active TCE (filled square) or CD3-PDD NCL (filled triangle, up and down). Masking efficiencies of the Binders #3 and #4 in the context of C7v122 CD3-binding domain are higher than for constructs shown in A), in line with the affinities shown in FIG. 2.

FIG. 4: Standard T-cell activation assay with active TCE (containing C7v14 CD3-binding domain (SEQ ID NO: 13)) and CD3-PDD NCL (containing C7v14 CD3-binding domain (SEQ ID NO: 13) and Binder #3 (SEQ ID NO: 3)), using EGFR-high expressing A431 tumor cells with approx. 200 k antigen binding sites (ABS) per cell (EGFR⁺⁺⁺) and EGFR-mid expressing HCT 116 tumor cells with approx. 20 k ABS/cell (EGFR⁺). The masking efficiency of the CD3-PDD NCL compared to the active TCE is much higher for EGFR⁺ HCT 116 cells (empty triangle vs empty square) compared to EGFR⁺⁺⁺ A431 tumor cells (filled triangle vs filled square).

FIG. 5: CD3-PDD NCL (triangle) and two CD3-PDD containing different cleavable linkers (CL, black and grey circle, respectively) without addition of matriptase (left part of gel) and after addition of matriptase and incubation for 24 h at 37° C. (right side of gel). CD3-PDD NCL with a non-cleavable linker is not impacted by addition of matriptase, whereas the two CD3-PDD CL are virtually fully cleaved into active TCE (2 domains; 2D) and Binder (1 domain; 1D). The bands of the full length 3-domain CD3-PDD (3D), 2-domain active TCE (2D) and 1-domain cleaved-off Binder (1D) are indicated.

FIG. 6: Cleavage rates of non-cleavable CD3-PDD NCL and of three different cleavable CD3-PDD CL that differ in their cleavable linker sequence. Cleavage rate was investigated at substrate concentrations of 2.5 μM for five different recombinant tumor-associated proteases (Matriptase, Urokinase, matrix metalloproteinase-2 (MMP-2), matrix metalloproteinase-7 (MMP-7), matrix metalloproteinase-9 (MMP-9)) at 37° C. and is indicated as cleavage rate (1/min).

FIG. 7: Standard tumor cell killing (A) and T-cell activation (B) assays using active TCE (square), non-cleavable CD3-PDD NCL (triangle) and cleavable CD3-PDD CL (circle) against HCT 116 wild-type (wt) or HCT 116 EGFR-knockout (KO) cells as target cells. Potent tumor cell killing and T-cell activation is observed for active TCE on HCT 116 wt cells, and to a lesser extent for CD3-PDD CL, presumably due to cleavage of CD3-PDD CL by proteases secreted by HCT 116 cells (see FIG. 7). Non-cleavable CD3-PDD NCL shows no tumor cell king or T-cell activation with HCT 116 wt cells. In absence of TAA (HCT 116 KO cells) no tumor cell killing or T-cell activation could be observed for any of the constructs.

FIG. 8: Immunoprecipitation-Western blot of non-cleavable and cleavable CD3-PDD (two different cleavable linkers #2 and #3, see also FIG. 6) samples at the end of a T-cell activation assay (48 h) using pan T-cells and A431 tumor cells. Samples containing the highest concentration of compound (10 nM) were immunoprecipitated and visualized on a Western blot with detection via anti-DARPin antibodies. CD3-PDD NCL showed no cleavage at the end of the assay, whereas the two different cleavable constructs exhibited different degree of cleavage, corresponding roughly with the respective T-cell activation masking window of the two constructs depicted on the left graph.

FIG. 9: Effect on masking efficiency of an α-HSA binding domain linked at the C-terminal end of the CD3-PDD NCL, determined by a standard tumor cell killing assay using pan T-cells and HCT 116 tumor cells. Masking efficiency between active TCE (square) and CD3-PDD NCL (triangle) or half-life extended (HLE) CD3-PDD NCL (diamond) is almost unchanged in constructs shown in Figure A). This figure depicts CD3-PDD constructs with Binder #1 (SEQ ID NO: 1) which exhibits high affinity (<1 nM K_{D}) towards CD3-binding domain. B) Constructs containing lower affinity Binder #3 (SEQ ID NO: 3) (>100 nM K_{D}) show a minor reduction of masking efficiency upon addition of an α-HSA binding domain to the C-terminus of the CD3-PDD NCL.

FIG. 10: Tumor cell binding (HCT 116 cells), T-cell binding (Jurkat cells), T-cell activation and tumor cell killing in vitro data (HCT 116 cells and pan T-cells) of constructs that were utilized for in vivo experiments (shown in FIG. 11). Data was generated for active TCE (square), non-cleavable CD3-PDD NCL (triangle), cleavable CD3-PDD CL (circle) and pre-cleaved CD3-PDD CL (half-filled circle) either containing the CD3-binding domain C7v119 (SEQ ID NO: 15) (lower affinity for CD3) or C7v122 (SEQ ID NO: 16) (higher affinity for CD3). For all CD3-PDD constructs Binder #4 (SEQ ID NO: 4) was used to mask the CD3-binding domain C7v119 or C7v122. T-cell activation and tumor cell killing for cleavable CD3-PDD CL shows a reduced masking compared to CD3-PDD NCL, which is due to the cleavage of the construct by proteases secreted by the tumor cells (see FIG. 8). The activation and killing data shown are representative experiments that were performed three times with pan T-cells from three individual human donors.

FIG. 11: In vivo experiment of active TCE, cleavable CD3-PDD CL and non-cleavable CD3-PDD NCL in a humanized mouse model (humanized with hematopoietic stem cells (CD34+) from human cord blood), engrafted with HCT 116 tumor ceils. The model was chosen for both anti-tumor efficacy and safety readout, as the EGFR-binding domain of the constructs is human-mouse cross-reactive and was shown to elicit strong toxicity in hHSC-humanized mice. A) Study design of 4 groups with each 6 mice, treated daily (vehicle control, CD3-PDD CL and CD3-PDD NCL) or intermittently (active TCE) due to strong toxicity findings. All constructs are non-half-life extended. B) Mean tumor growth curves for the four groups, with treatments indicated at the top by black arrows for each group. Error bars depict SEM values with n=6 except for group #2 (active TCE), where 2 mice were lost on day 12 and 1 mouse on day 13 due to strong toxicity. C) Individual tumor growth curves of the four groups, with treatments indicated by black arrows above each graph. D) Body weight (BW) change over the course of the experiment, with the average BW of each group set to 100% at the day before the first injection. Treatment for each group is indicated at the top of the graph, as well as the initiation and end of treatment. A hash symbol depicts animals that died or had to be sacrificed for humane reasons due to bad clinical score. E) Average clinical health score (scoring of different clinical signs as observed by the experimenter) of each group, with treatment of each group indicated at the top of the graph by arrows. Darker fields indicate more severe (i.e. worse) clinical health scores. F) Human cytokines (TNF-α, IFN-γ, IL-2 and IL-6) before tumor cell engraftment (pre-dose/basal) and 4 h after first treatment. Elevated levels of cytokines were only observed for the active TCE, whereas the CD3-PDD NCL and CD3-PDD CL both showed no significantly elevated cytokine levels. Each treated group was compared to the vehicle control (***, p<0.001).

### DETAILED DESCRIPTION OF THE INVENTION

Overview

This application relates to prodrugs comprising a drug molecule connected by a protease-cleavable peptide linker to a binder, which reversibly inhibits biological activity of the drug molecule, and to the inhibitory binders themselves. Also described are nucleic acids encoding the recombinant proteins described herein, and methods of making said recombinant proteins, as well as methods of treatment and medical uses of the recombinant proteins.

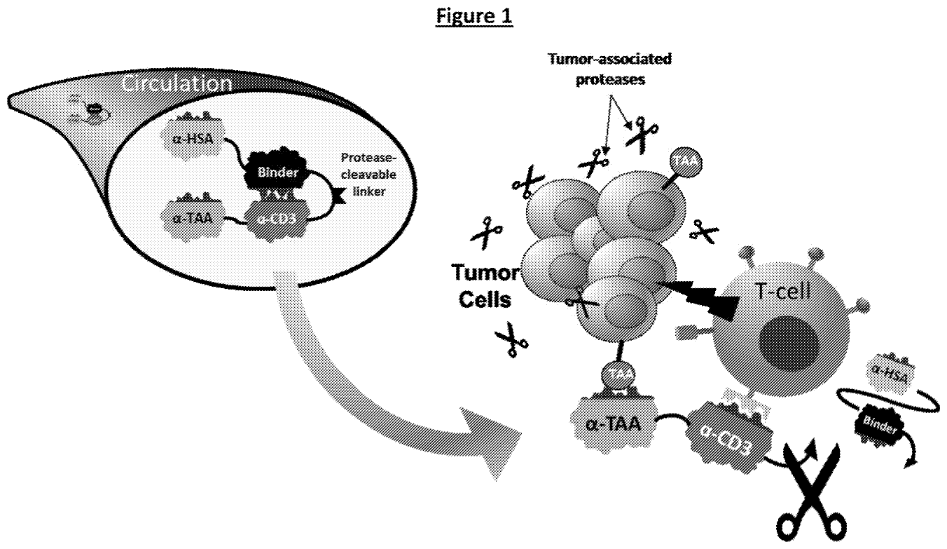

The protease-cleavable prodrug of the invention comprises a binding moiety. e.g. an anti-idiotypic binding moiety, and a drug molecule, linked by a protease-cleavable linker. The binding moiety reversibly binds to the drug molecule and, when bound, inhibits a biological activity of the drug molecule. Upon administration in vivo, proteases (e.g. proteases present in tumor tissue) cleave the linker between the binding moiety and the drug molecule, releasing the drug molecule into the body (see FIG. 1). Conditional release of active drug molecule upon administration can minimise adverse effects, or the risk thereof, otherwise associated with the drug molecule.

Without wishing to be bound by theory, it is understood that the binding moieties described herein inhibit the biological activity (i.e., mode of action) of a drug molecule when bound to it, such as when the binding moiety is connected to the drug molecule by a peptide linker. Upon administration to a patient, proteases, such as proteases present in tumor tissue, can cleave the peptide linker between the binding moiety and the drug molecule, leading to release of active drug molecule due to dissociation and diffusion away of the binding moiety from the drug molecule.

The invention relates to recombinant proteins comprising a drug molecule and a binding moiety, connected by a protease-cleavable linker, and to the binding moieties themselves.

The invention further relates to pharmaceutical compositions comprising said prodrugs and to use of said compositions in therapy. For example, the present invention relates to use of such compositions in the treatment of proliferative diseases, such as cancer.

The invention further relates to nucleic acids encoding said binding moieties and methods of making these using host cells.

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry described herein are those well-known and commonly used in the art.

The terms “comprising”, “having”, “including” and “containing” are to be construed as open-ended terms unless otherwise noted. If aspects of the invention are described as “comprising” a feature, embodiments also are contemplated “consisting of” or “consisting essentially of” the feature. The use of any and all examples, or exemplary language (e.g., “such as”) provided herein, is intended merely to better illustrate the disclosure and does not pose a limitation on the scope of the disclosure unless otherwise claimed. No language in the specification should be construed as indicating any non-claimed element as essential to the practice of the disclosure. Other than in the operating examples, or where otherwise indicated, all numbers expressing quantities of ingredients or reaction conditions used herein should be understood as modified in all instances by the term “about” as that term would be interpreted by the person skilled in the relevant art. The term “about” as used herein is equivalent to ±10% of a given numerical value, unless otherwise stated.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

In the context of the present invention the term “protein” refers to a molecule comprising a polypeptide, wherein at least part of the polypeptide has, or is able to acquire, a defined three-dimensional arrangement by forming secondary, tertiary, and/or quaternary structures within a single polypeptide chain and/or between multiple polypeptide chains. If a protein comprises two or more polypeptide chains, the individual polypeptide chains may be linked non-covalently or covalently, e.g., by a disulfide bond between two polypeptides. A part of a protein, which individually has, or is able to acquire, a defined three-dimensional arrangement by forming secondary and/or tertiary structure, is termed “protein domain”. Such protein domains are well known to the practitioner skilled in the art.

The term “drug molecules” (used interchangeably herein with the term “drugs”) refers to therapeutic agents that comprise a polypeptide or a protein, wherein said polypeptide or protein contains a site that is capable of being bound by a binding moiety. Preferred drug molecules for use in the present invention are T-cell engager or TCE drug molecules.

The term “recombinant” as used in recombinant protein, recombinant polypeptide and the like, means that said protein or polypeptide is produced by the use of recombinant DNA technologies well known to the practitioner skilled in the art. For example, a recombinant DNA molecule (e.g., produced by gene synthesis) encoding a polypeptide can be cloned into a bacterial expression plasmid (e.g. pQE30, QIAgen), yeast expression plasmid, mammalian expression plasmid, or plant expression plasmid, or a DNA enabling in vitro expression. If, for example, such a recombinant bacterial expression plasmid is inserted into appropriate bacteria (e.g., Escherichia coli), these bacteria can produce the polypeptide(s) encoded by this recombinant DNA. The correspondingly produced polypeptide or protein is called a recombinant polypeptide or recombinant protein. A recombinant polypeptide or recombinant protein can also be expressed from other nucleic acid molecules, such as mRNA encoding said polypeptide or protein.

In the context of the present invention, the term “binding moiety” or “binder” refers to a binding agent that comprises a polypeptide or a protein, wherein said polypeptide or protein is capable of non-covalently binding to a drug molecule. The binding moiety does not necessarily need to bind to an active site of the drug molecule. The binding moiety must, however, bind in such a way as to inhibit the mode of action of the drug. This may be by binding to the active site of the drug but may also be by binding to another site on the drug molecule to either change the conformation of said drug (i.e., allosteric inhibition), or to sterically hinder the active site of the drug molecule. The active site of the drug molecule is involved in a biological activity of the drug molecule. The biological activity can be an enzymatic activity or binding to a biological target. Binding of a binding moiety to a drug molecule inhibits a biological activity of the drug molecule. For example, binding of a binding moiety to a drug molecule inhibits the enzymatic activity of the drug molecule or inhibits binding of the drug molecule to a biological target. Binding of a binding moiety to a drug molecule may be anti-idiotypic. Thus, a binding moiety may be an anti-idiotypic binder of a drug molecule.

The binding moieties used in the present invention include antibodies, alternative scaffolds, and polypeptides. As used herein, the term “antibody” refers not only to intact antibody molecules, such as those typically produced by the immune system when it detects foreign antigens, but also to any fragments, variants and synthetic or engineered analogues of antibody molecules that retain antigen-binding ability. Such fragments, variants and analogues are also well known in the art and are regularly employed in vitro or in vivo. Accordingly, the term “antibody” encompasses intact immunoglobulin molecules, antibody fragments such as, e.g., Fab, Fab′, F(ab′)2, and single chain V region fragments (scFv), bispecific or multispecific antibodies, chimeric antibodies, humanized antibodies, antibody fusion proteins, unconventional antibodies, and proteins comprising an antigen binding domain derived from an immunoglobulin molecule. As used herein, the term “alternative scaffolds” refers to any molecule comprising or consisting of a protein, but that is not an antibody.

A binding moiety of any of these different structural types can bind to a drug molecule, and, when bound, inhibit the mode of action of the drug molecule. In one preferred embodiment, a binding moiety comprises an ankyrin repeat domain with binding specificity for a drug molecule. In another preferred embodiment, a binding moiety comprises an antibody with binding specificity fora drug molecule. In another preferred embodiment, a binding moiety comprises an alternative scaffold with binding specificity for a drug molecule, wherein the alternative scaffold does not comprise an ankyrin repeat domain. In one preferred embodiment, the drug molecule comprises an antibody and the binding moiety is an anti-idiotypic antibody with binding specificity for said antibody comprised in said drug molecule. In another preferred embodiment, the drug molecule comprises an antibody and the binding moiety is an anti-idiotypic alternative scaffold, such as, e.g., an ankyrin repeat domain, with binding specificity for said antibody comprised in said drug molecule. In another preferred embodiment, the drug molecule comprises an alternative scaffold, such as, e.g., an ankyrin repeat domain, and the binding moiety is an anti-Idiotypic antibody with binding specificity for said alternative scaffold comprised in said drug molecule. In another preferred embodiment, the drug molecule comprises an alternative scaffold, such as, e.g., an ankyrin repeat domain, and the binding moiety is an anti-Idiotypic alternative scaffold, such as, e.g., an ankyrin repeat domain, with binding specificity for said alternative scaffold comprised in said drug molecule.

The term “nucleic acid” or “nucleic acid molecule” refers to a polynucleotide molecule, which may be a ribonucleic acid (RNA) or deoxyribonucleic acid (DNA) molecule, either single stranded or double stranded, and includes modified and artificial forms of DNA or RNA. A nucleic acid molecule may either be present in isolated form or be comprised in recombinant nucleic acid molecules or vectors.

The term “biological target” refers to an individual molecule such as a nucleic acid molecule, a polypeptide or protein, a carbohydrate, or any other naturally occurring molecule, including any part of such individual molecule, or to complexes of two or more of such molecules, or to a whole cell or a tissue sample, or to any non-natural compound. Preferably, a target is a naturally occurring or non-natural polypeptide or protein, or a polypeptide or protein containing chemical modifications, for example, naturally occurring or non-natural phosphorylation, acetylation, or methylation. In some embodiments, the biological target is an immune cell, such as a T cell, a B cell, a natural killer (NK) cell, or another type of immune cell. In some other embodiments, the biological target is a tumor cell.

In the context of the present invention, the term “polypeptide” relates to a molecule consisting of a chain of multiple, i.e., two or more, amino acids linked via peptide bonds. Preferably, a polypeptide consists of more than eight amino acids linked via peptide bonds. The term “polypeptide” also includes multiple chains of amino acids, linked together by S—S bridges of cysteines. Polypeptides are well-known to the person skilled in the art.

Patent application WO2002/020565 and Forrer et al., 2003 (Forrer, P., Stumpp, M. T., Binz, H. K., Plückthun, A., 2003. FEBS Letters 539, 2-6), contain a general description of repeat protein features and repeat domain features, techniques and applications. The term “repeat protein” refers to a protein comprising one or more repeat domains. Preferably, a repeat protein comprises one, two, three, four, five or six repeat domains. Furthermore, said repeat protein may comprise additional non-repeat protein domains, polypeptide tags and/or peptide linkers. The repeat domains can be binding domains. The term “repeat domain” refers to a protein domain comprising two or more consecutive repeat modules as structural units, wherein said repeat modules have structural and sequence homology. Preferably, a repeat domain also comprises an N-terminal and/or a C-terminal capping module. For clarity, a capping module can be a repeat module. Such repeat domains, repeat modules, and capping modules, sequence motives, as well as structural homology and sequence homology are well known to the practitioner in the art from examples of ankyrin repeat domains (Binz et al., J. Mol. Biol. 332, 489-503, 2003; Binz et al., Nature Biotech. 22(5): 575-582 (2004); WO20021020565; WO2012/069655), leucine-rich repeat domains (WO2002/020565), tetratricopeptide repeat domains (Main, E. R., Xiong, Y., Cocco, M. J., D'Andrea, L., Regan, L., Structure 11(5), 497-508, 2003), and armadillo repeat domains (WO2009/040338). It is further well known to the practitioner in the art, that such repeat domains are different from proteins comprising repeated amino acid sequences, where every repeated amino acid sequence is able to form an individual domain (for example FN3 domains of Fibronectin).

The term “ankyrin repeat domain” refers to a repeat domain comprising two or more consecutive ankyrin repeat modules as structural units. Ankyrin repeat domains may be modularly assembled into larger ankyrin repeat proteins, optionally with half-life extension domains, using standard recombinant DNA technologies (see, e.g., Forrer, P., et al., FEBS letters 539, 2-6, 2003, WO2002/020565, WO2016/156596, WO2018/054971).

The term “designed” as used in designed ankyrin repeat protein and designed ankyrin repeat domain and the like refers to the property that such repeat proteins and repeat domains, respectively, are man-made and do not occur in nature. The designed repeat proteins described herein comprise at least one designed repeat domain. Preferably, the designed repeat domain is a designed ankyrin repeat domain.

The term “target interaction residues” refers to amino acid residues of a binding moiety which contribute to the direct interaction with a drug molecule. For example, if a binding moiety is a designed ankyrin repeat domain, then the term “target interaction residues” refers to amino acid residues of the designed ankyrin repeat domain which contribute to the direct interaction with a drug molecule.

The terms “framework residues” or “framework positions” refer to amino acid residues of a repeat module, which contribute to the folding topology, i.e., which contribute to the fold of said repeat module or which contribute to the interaction with a neighbouring module. Such contribution may be the interaction with other residues in the repeat module, or the influence on the polypeptide backbone conformation as found in α-helices or O-sheets, or the participation in amino acid stretches forming linear polypeptides or loops. Such framework and target interaction residues may be identified by analysis of the structural data obtained by physicochemical methods, such as X-ray crystallography, NMR and/or CD spectroscopy, or by comparison with known and related structural information well known to practitioners in structural biology and/or bioinformatics.

The term “repeat modules” refers to the repeated amino acid sequence and structural units of designed repeat domains, which are originally derived from the repeat units of naturally occurring repeat proteins. Each repeat module comprised in a repeat domain is derived from one or more repeat units of a family or subfamily of naturally occurring repeat proteins, preferably the family of ankyrin repeat proteins. Furthermore, each repeat module comprised in a repeat domain may comprise a “repeat sequence motif” deduced from homologous repeat modules obtained from repeat domains selected on a target and having the same target specificity.

Accordingly, the term “ankyrin repeat module” refers to a repeat module, which is originally derived from the repeat units of naturally occurring ankyrin repeat proteins. Ankyrin repeat proteins are well known to the person skilled in the art. Designed ankyrin repeat proteins have been described previously; see, e.g., International Patent Publication Nos. WO2002/020565, WO2010/060748, WO2011/135067, WO2012/069654, WO2012/069655, WO2014/001442, WO2014/191574. WO2014/083208, WO2016/156596, and WO2018/054971, all of which are incorporated by reference in their entireties. Typically, an ankyrin repeat module comprises about 31 to 33 amino acid residues that form two alpha helices, separated by loops.

Repeat modules may comprise positions with amino acid residues which have not been randomized in a library for the purpose of selecting target-specific repeat domains (“non-randomized positions” or “fixed positions” used interchangeably herein) and positions with amino acid residues which have been randomized in the library for the purpose of selecting target-specific repeat domains (“randomized positions”). The non-randomized positions comprise framework residues. The randomized positions comprise target interaction residues. “Have been randomized” means that two or more amino acids were allowed at an amino acid position of a repeat module, for example, wherein any of the usual twenty naturally occurring amino acids were allowed, or wherein most of the twenty naturally occurring amino acids were allowed, such as amino acids other than cysteine, or amino acids other than glycine, cysteine and proline.

The term “repeat sequence motif” refers to an amino acid sequence, which is deduced from one or more repeat modules. Preferably, said repeat modules are from repeat domains having binding specificity for the same target. Such repeat sequence motifs comprise framework residue positions and target interaction residue positions. Said framework residue positions correspond to the positions of framework residues of the repeat modules. Likewise, said target interaction residue positions correspond to the positions of target interaction residues of the repeat modules. Repeat sequence motifs comprise non-randomized positions and randomized positions.

The term “repeat unit” refers to amino acid sequences comprising sequence motifs of one or more naturally occurring proteins, wherein said “repeat units” are found in multiple copies and exhibit a defined folding topology common to all said motifs determining the fold of the protein. Examples of such repeat units include leucine-rich repeat units, ankyrin repeat units, armadillo repeat units, tetratricopeptide repeat units. HEAT repeat units, and leucine-rich variant repeat units.

A binding moiety “specifically binds” or “preferentially binds” (used interchangeably herein) to a drug molecule if it reacts or associates more frequently, more rapidly, with greater duration, with greater affinity and/or with greater avidity with a particular drug molecule than it does with alternative targets (e.g., cells or substances). Binding moieties can be tested for specificity of binding by comparing binding to an appropriate drug molecule to binding to an alternate drug molecule under a given set of conditions. In some embodiments, if the binding molecule binds to the appropriate drug molecule with at least 2-fold, at least 5-fold, at least 10-fold, at least 100-fold, or at least 1000-fold higher affinity than to the alternate drug molecule, then it is considered to be specific. It is also understood by reading this definition that, for example, a binding moiety which specifically or preferentially binds to a first drug molecule may or may not specifically or preferentially bind to a second drug molecule. As such, “specific binding” does not necessarily require (although it can include) exclusive binding. In general, under designated assay conditions, a binding moiety binds preferentially to a particular drug molecule and does not bind in a significant amount to other components present in a test sample.

A variety of assay formats may be used to select or characterize a binding moiety that specifically binds a drug molecule of interest. For example, solid-phase ELISA immunoassay, immunoprecipitation, BIAcore™ (GE Healthcare, Piscataway, NJ), fluorescence-activated cell sorting (FACS), Octet™ (ForteBio, Inc., Menlo Park, CA) and Western blot analysis are among many assays that may be used to identify a binding moiety that specifically binds to a target drug molecule. Typically, a specific or selective binding wilt be at least twice the background signal or noise and more typically more than 10 times the background signal. More particularly, a binding moiety is said to “specifically bind” a target when the equilibrium dissociation constant (K_{D}) value is <1 μM, such as <500 nM, <100 nM, <10 nM, <1 nM, <100 pM or <10 pM.

A variety of methods of measuring binding affinity are known in the art, any of which can be used for purposes of the present invention. For example, as exemplified herein, the binding affinity of a particular binding moiety to a drug molecule target can be expressed as K_{D} value, which refers to the dissociation constant of the binding moiety and the drug molecule target. K_{D} is the ratio of the rate of dissociation, also called the “off-rate (k_{off})”, to the association rate, or “on-rate (kₒₙ)”. Thus, K_{D} equals k_{off}/kₒₙ and is expressed as a molar concentration (M), and the smaller the K_{D}, the stronger the affinity of binding.

K_{D} values can be determined using any suitable method. One exemplary method for measuring K_{D} is surface plasmon resonance (SPR) (see. e.g., Nguyen et al. Sensors (Basel). 2015 May 5; 15(5):10481-510). K_{D} value may be measured by SPR using a biosensor system such as a BIACORE® system. BIAcore kinetic analysis comprises, e.g., analysing the binding and dissociation of an antigen from chips with immobilized molecules (e.g., molecules comprising epitope binding domains), on their surface.

Another method for determining the K_{D} of a protein is by using Bio-Layer Interferometry (see, e.g., Shah et al. J Vis Exp. 2014; (84): 51383). K_{D} value may be measured using OCTET® technology (Octet QKe system, ForteBio). Alternatively, or in addition, a KinExA® (Kinetic Exclusion Assay) assay, available from Sapidyne Instruments (Boise, Id.) can also be used. Any method suitable for assessing the binding affinity between two binding partners is encompassed herein. Surface plasmon resonance (SPR) is particularly preferred. Most preferably, the K_{D} values are determined in PBS and by SPR.

The term “PBS” means a phosphate buffered water solution containing 137 mM NaCl, 10 mM phosphate and 2.7 mM KCl and having a pH of 7.4.

The term “treat,” as well as words related thereto, does not necessarily imply 100% or complete cure. Rather, there are varying degrees of treatment of which one of ordinary skill in the art recognizes as having a potential benefit or therapeutic effect. In this respect, the methods of treatment and medical uses described herein can provide any amount or any level of treatment. Furthermore, the treatment provided by the method of the present disclosure can include treatment of (i.e., relief from) one or more conditions or symptoms. In exemplary aspects, the invention provides methods of treatment with a prodrug molecule comprising the administration of the prodrug molecule to a patient, wherein the adverse effects, or the risk thereof, experienced by the patient are reduced compared to the adverse effects, or the risk thereof, the patient would experience if the same amount of drug molecule was administered without being in a prodrug form (i.e., not connected to a binding moiety of the invention by a protease-cleavable linker). Thus, the use of a binding moiety of the invention to form a prodrug molecule allows methods of treatment with reduced adverse effects, or a reduced risk thereof, and/or methods of treatment with a higher dose of the drug molecule or with administration of the drug molecule over a shorted period of time.

Therapeutic responses in any given disease or condition can be determined by standardized response criteria specific to that disease or condition. The subject undergoing therapy may experience the beneficial effect of an improvement in the symptoms associated with the disease, together with a reduction in adverse effects, or the risk thereof, associated with administration of the therapeutic agent.

As used herein, the term “proliferative disease” refers to diseases characterised by excessive production of cells. Examples of proliferative diseases include, but are not limited to, cancer, atherosclerosis, rheumatoid arthritis, psoriasis, idiopathic pulmonary fibrosis, scleroderma and cirrhosis of the liver. In a preferred embodiment, the proliferative disease is cancer.

Binding Moiety

The binding moieties described herein are polypeptides or proteins with a variety of different structures, which can specifically bind to a drug molecule. Examples of binding moieties for use in the present invention include antibodies, alternative scaffolds, and polypeptides.

Antibodies Include any polypeptides or proteins comprising an antigen binding domain that is derived from an antibody or immunoglobulin molecule. The antigen binding domain can be derived, for example, from monoclonal antibodies, polyclonal antibodies, recombinant antibodies, human antibodies, humanized antibodies, and single-domain antibodies, e.g., a heavy chain variable domain (VH), a light chain variable domain (VL) and a variable domain (VHH) from, e.g., human or camelid origin. In some instances, it is beneficial for the antigen binding domain to be derived from the same species in which the binding moiety will ultimately be used in. For example, for use in humans, it may be beneficial for the antigen binding domain of the binding moiety described herein, to comprise a human or a humanized antigen binding domain. Antibodies can be obtained using techniques well known in the art.

In one embodiment, the binding moiety is a camelid nanobody. Camelid nanobodies (also known as camelid single-domain antibodies or VHHs) are derived from the Camelidae family of mammals such the llamas, camels, and alpacas. Unlike other antibodies, camelid antibodies lack a light chain and are composed of two identical heavy chains. Camelid antibodies typically have a relatively low molecular weight in the region of around 15 kDa.

In one embodiment, the binding moiety is a shark antibody domain. Shark antibody domains, like camelid nanobodies, also lack a light chain.

Alternative scaffolds include any polypeptides or proteins comprising a binding domain that is capable of binding an antigen (such as a drug molecule) and that is not derived from an antibody or immunoglobulin molecule. The binding domain of alternative scaffolds may comprise or may be derived from a variety of different polypeptide or protein structures. Alternative scaffolds include, but are not limited to, adnectins (monobodies), affibodies, affilins, affimers and aptamers, affitins, alphabodies, anticalins, armadillo repeat protein-based scaffolds, atrimers, avimers, ankyrin repeat protein-based scaffolds (such as DARPin® proteins), fynomers, knottins, and Kunitz domain peptides. Alternative scaffolds are described, e.g., in Yu et al., Annu Rev Anal Chem (Palo Alto Calif). 2017 Jun. 12; 10(1): 293-320. doi:10.1146/annurevanchem-061516-045205.

Adnectins are originally derived from the tenth extracellular domain of human fibronectin type III protein (10Fn3). The fibronectin type III domain has 7 or 8 beta strands, which are distributed between two beta sheets, which themselves pack against each other to form the core of the protein, and further contain loops (analogous to CDRs), which connect the beta strands to each other and are solvent exposed. There are at least three such loops at each edge of the beta sheet sandwich, where the edge is the boundary of the protein perpendicular to the direction of the beta strands (see U.S. Pat. No. 6,818,418). Because of this structure, this non-antibody scaffold mimics antigen binding properties that are similar in nature and affinity to those of antibodies. These scaffolds can be used in a loop randomization and shuffling strategy in vitro that is similar to the process of affinity maturation of antibodies in vivo.

Affibody affinity ligands are composed of a three-helix bundle based on the scaffold of one of the IgG-binding domains of Protein A, which is a surface protein from the bacterium Staphylococcus aureus. This scaffold domain consists of 58 amino acids, 13 of which are randomized to generate affibody libraries with a large number of ligand variants (See e.g., U.S. Pat. No. 5,831,012). Affibody molecules mimic antibodies, but are considerably smaller, having a molecular weight of around 6 kDa, compared to around 150 kDa for antibodies. Despite the size difference, the binding site of affibody molecules has similarity to that of an antibody.

Affilins are synthetic antibody mimetics that are structurally derived from human ubiquitin (historically also from gamma-B crystallin). Affilins consists of two identical domains with mainly beta sheet structure and a total molecular mass of about 20 kDa. They contain several surface-exposed amino acids that are suitable for modification. Affilins resemble antibodies in their affinity and specificity to antigens but not in structure.

Affimers are a type of peptide aptamer, having a structure known as SQT (Stefin A quadruple mutant-Tracy). Aptamers and affimers are short peptides responsible for affinity binding with an inert and rigid protein scaffold for structure constraining in which both N- and C-termini of the binding peptide are embedded in the inert scaffold.

Affitins are variants of the DNA binding protein Sac7d that are engineered to obtain specific binding affinities. Sac7d is originally derived from the hyperthermophile archaea Sulfolobus acidocaldarius and binds with DNA to prevent it from thermal denaturation. Affitins are commercially known as Nanofitins.

Alphabodies are small (approximately 10 kDa) proteins that are engineered to bind to a variety of antigens and are therefore antibody mimetics. The alphabody scaffold is computationally designed based on coiled-coil structures. The standard alphabody scaffold contains three α-helices, composed of four heptad repeats (stretches of 7 residues) each, connected via glycine/serine-rich linkers. The standard heptad sequence is “IAAIQKQ”. Alphabodies' ability to target extracellular and intracellular proteins in combination with their high binding affinities may allow them to bind to targets that cannot be reached with antibodies.

Anticalins are a group of binding proteins with a robust and conservative n-barrel structure found in lipocalins. Lipocalins are a class of extracellular proteins comprising one peptide chain (150-190 amino acids) that is in charge of recognition, storage, and transport of various biological molecules such as signalling molecules.

Armadillo repeat protein-based scaffolds are abundant in eukaryotes and are involved in a broad range of biological processes, especially those related to nuclear transport. Armadillo repeat protein-based scaffolds usually consist of three to five internal repeats and two capping elements. They also have a tandem elongated superhelical structure that enables binding with their corresponding peptide ligands in an extended conformation.

Atrimers are a scaffold derived from a trimeric plasma protein known as tetranectin, belonging to a family of C-type lectins consisting of three identical units. The structure of the C-type lectin domain (CTLD) within the tetranectin has five flexible loops that mediate interaction with targeting molecules.

Avimers are derived from natural A-domain containing proteins such as HER3 and consist of a number of different “A-domain” monomers (2-10) linked via amino acid linkers. Avimers can be created that can bind to the target antigen using the methodology described in, for example, U.S. Patent Application Publication Nos. 2004/0175756; 2005/0053973; 2005/0048512; and 2006/0008844.

In one embodiment, the binding moiety is an ankyrin repeat protein. Designed or engineered ankyrin repeat proteins (such as DARPin® proteins) can function like antibody mimetic proteins, typically exhibiting highly specific and high-affinity target binding. Designed ankyrin repeat proteins comprise one or more designed ankyrin repeat domains. Designed ankyrin repeat domains are derived from natural ankyrin repeat proteins and each designed ankyrin repeat domain typically binds a target protein with high specificity and affinity. Due to their high specificity, stability, potency and affinity and due to their flexibility in formatting to generate mono-, bi- or multi-specific proteins, designed ankyrin repeat proteins are particularly suitable for use as high-affinity binding moieties. Designed ankyrin repeat protein drug candidates also display favourable development properties including rapid, low-cost and high-yield manufacturing and up to several years of shelf-life at 4° C. Designed ankyrin repeat proteins are a preferred embodiment of binding moieties of the invention. DARPin® is a registered trademark owned by Molecular Partners AG.

Fynomers are small globular proteins (approximately 7 kDa) that evolved from amino acids 83-145 of the Src homology domain 3 (SH3) of the human Fyn tyrosine kinase. Fynomers are attractive binding molecules due to their high thermal stability, cysteine-free scaffold, and human origin, which reduce potential immunogenicity.

Knottins, also known as cysteine knot miniproteins, are typically proteins 30 amino acids in length comprising three antiparallel β-sheets and constrained loops laced by a disulfide bond, which creates a cysteine knot. This disulfide bond confers high thermal stability making knottins attractive antibody mimetics.

Kunitz domain peptides or Kunitz domain inhibitors are a class of protease inhibitors with irregular secondary structures containing ˜60 amino acids with three disulfide bonds and three loops that can be mutated without destabilizing the structural framework.

In one embodiment, the binding moiety is a polypeptide or protein comprising an antigen binding domain derived from a T cell receptor (TCR).

Examples of Binding Moieties

Examples of ankyrin repeat domains for use as binding moieties in the present invention are provided by SEQ ID NOs: 1 to 12.

The ankyrin repeat domains of SEQ ID NOs:1 to 12 specifically bind to a CD3-specific binding molecule having an amino acid sequence selected from SEQ ID NOs: 13 to 17. For example, the ankyrin repeat domains of SEQ ID NOs: 1 to 12 specifically bind to a CD3-specific binding molecule having the amino acid sequence of SEQ ID NO: 13 or the amino acid sequence of SEQ ID NO: 14 or the amino acid sequence of SEQ ID NO: 15 or the amino acid sequence of SEQ ID NO: 16 or the amino acid sequence of SEQ ID NO: 17.

In one embodiment, the ankyrin repeat domains of SEQ ID NOs: 1 to 12 specifically bind to a CD3-specific binding molecule having the amino acid sequence of SEQ ID NO: 13. In another embodiment, the ankyrin repeat domains of SEQ ID NOs: 1 to 12 specifically bind to a CD3-specific binding molecule having the amino acid sequence of SEQ ID NO: 14. In another embodiment, the ankyrin repeat domains of SEQ ID NOs: 1 to 12 specifically bind to a CD3-specific binding molecule having the amino acid sequence of SEQ ID NO: 15. In another embodiment, the ankyrin repeat domains of SEQ ID NOs: 1 to 12 specifically bind to a CD3-specific binding molecule having the amino acid sequence of SEQ ID NO: 16. In another embodiment, the ankyrin repeat domains of SEQ ID NOs: 1 to 12 specifically bind to a CD3-specific binding molecule having the amino acid sequence of SEQ ID NO: 17.

Thus, in one embodiment, the binding moiety of the invention is an ankyrin repeat domain comprising an amino acid sequence that has at least about 85% sequence identity with an ankyrin repeat domain selected from the group consisting of SEQ ID NOs: 1 to 12.

In one embodiment, the binding moiety is an ankyrin repeat domain comprising an amino acid sequence that has at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% sequence identity with an ankyrin repeat domain selected from the group consisting of SEQ ID NOs: 1 to 12.

In one embodiment, the binding moiety is an ankyrin repeat domain, wherein said ankyrin repeat domain is selected from the group consisting of SEQ ID NOs: 1 to 12.

In one embodiment, the binding moiety is a designed ankyrin repeat domain comprising an amino acid sequence selected from the group consisting of (1) SEQ ID NOs: 1 to 12 and (2) sequences that have at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% amino acid sequence identity with any of SEQ ID NOs: 1 to 12.

In one embodiment, the binding moiety is a designed ankyrin repeat protein comprising an ankyrin repeat module comprising an amino acid sequence selected from the group consisting of (1) SEQ ID NOs: 45 to 64 and (2) sequences in which up to 9 amino acids in any of SEQ ID NOs: 45 to 64 are substituted by another amino acid. In one embodiment, the binding moiety is a designed ankyrin repeat protein comprising an ankyrin repeat module comprising an amino acid sequence selected from the group consisting of (1) SEQ ID NOs: 45 to 64 and (2) sequences in which up to 8 amino acids in any of SEQ ID NOs: 45 to 64 are substituted by another amino acid. In one embodiment, the binding moiety is a designed ankyrin repeat protein comprising an ankyrin repeat module comprising an amino acid sequence selected from the group consisting of (1) SEQ ID NOs: 45 to 64 and (2) sequences in which up to 7 amino acids in any of SEQ ID NOs: 45 to 64 are substituted by another amino acid. In one embodiment, the binding moiety is a designed ankyrin repeat protein comprising an ankyrin repeat module comprising an amino acid sequence selected from the group consisting of (1) SEQ ID NOs: 45 to 64 and (2) sequences in which up to 6 amino acids in any of SEQ ID NOs: 45 to 64 are substituted by another amino acid. In one embodiment, the binding moiety is a designed ankyrin repeat protein comprising an ankyrin repeat module comprising an amino acid sequence selected from the group consisting of (1) SEQ ID NOs: 45 to 64 and (2) sequences in which up to 5 amino acids in any of SEQ ID NOs: 45 to 64 are substituted by another amino acid. In one embodiment, the binding moiety is a designed ankyrin repeat protein comprising an ankyrin repeat module comprising an amino acid sequence selected from the group consisting of (1) SEQ ID NOs: 45 to 64 and (2) sequences in which up to 4 amino acids in any of SEQ ID NOs: 45 to 64 are substituted by another amino acid. In one embodiment, the binding moiety is a designed ankyrin repeat protein comprising an ankyrin repeat module comprising an amino acid sequence selected from the group consisting of (1) SEQ ID NOs: 45 to 64 and (2) sequences in which up to 3 amino acids in any of SEQ ID NOs: 45 to 64 are substituted by another amino acid. In one embodiment, the binding moiety is a designed ankyrin repeat protein comprising an ankyrin repeat module comprising an amino acid sequence selected from the group consisting of (1) SEQ ID NOs: 45 to 64 and (2) sequences in which up to 2 amino acids in any of SEQ ID NOs: 45 to 64 are substituted by another amino acid. In one embodiment, the binding moiety is a designed ankyrin repeat protein comprising an ankyrin repeat module comprising an amino acid sequence selected from the group consisting of (1) SEQ ID NOs: 45 to 64 and (2) sequences in which up to 1 amino acid in any of SEQ ID NOs: 45 to 64 is substituted by another amino acid.

The amino acid sequences described herein may be substituted by one or more amino acids. In some embodiments, up to 15, up to 14, up to 13, up to 12, up to 11, up to 10, up to 9, up to 8, up to 7, up to 6, up to 5, up to 4, up to 3, up to 2, or up to 1 substitution is made in any of the binding moieties described herein.

In some embodiments, up to 15, up to 14, up to 13, up to 12, up to 11, up to 10, up to 9, up to 8, up to 7, up to 6, up to 5, up to 4, up to 3, up to 2, or up to 1 substitution is made in any ankyrin repeat domain relative to any of the sequences of SEQ ID NOs: 1 to 12. In some embodiments, up to 15 substitutions are made relative to any of the sequences of SEQ ID NOs: 1 to 12. In some embodiments, up to 14 substitutions are made relative to any of the sequences of SEQ ID NOs: 1 to 12. In some embodiments, up to 13 substitutions are made relative to any of the sequences of SEQ ID NOs: 1 to 12. In some embodiments, up to 12 substitutions are made relative to any of the sequences of SEQ ID NOs: 1 to 12. In some embodiments, up to 11 substitutions are made relative to any of the sequences of SEQ ID NOs: 1 to 12. In some embodiments, up to 10 substitutions are made relative to any of the sequences of SEQ ID NOs: 1 to 12. In some embodiments, up to 9 substitutions are made relative to any of the sequences of SEQ ID NOs: 1 to 12. In some embodiments, up to 8 substitutions are made relative to any of the sequences of SEQ ID NOs:1 to 12. In some embodiments, up to 7 substitutions are made relative to any of the sequences of SEQ ID NOs: 1 to 12. In some embodiments, up to 6 substitutions are made relative to any of the sequences of SEQ ID NOs: 1 to 12. In some embodiments, up to 5 substitutions are made relative to any of the sequences of SEQ ID NOs: 1 to 12. In some embodiments, up to 4 substitutions are made relative to any of the sequences of SEQ ID NOs: 1 to 12. In some embodiments, up to 3 substitutions are made relative to any of the sequences of SEQ ID NOs: 1 to 12. In some embodiments, up to 2 substitutions are made relative to any of the sequences of SEQ ID NOs: 1 to 12. In some embodiments, up to 1 substitution is made relative to any of the sequences of SEQ ID NOs: 1 to 12.

In some embodiments, the amino acid substitution(s) are all made in framework positions. In some embodiments, the amino acid substitution(s) are all made in non-randomized positions. The location of randomized positions in a designed ankyrin repeat domain is disclosed, e.g., in Binz et al., Nature Biotech. 22(5): 575-582 (2004).

In some embodiments, the amino acid substitution(s) do not change the K_{D} value by more than about 1000-fold, more than about 100-fold, or more than about 10-fold, compared to the K_{D} value of the unsubstituted binding moieties. For example, in some embodiments, the amino acid substitution(s) do not change the K_{D} value by more than about 1000-fold, more than about 300-fold, more than about 100-fold, more than about 50-fold, more than about 25-fold, more than about 10-fold, or more than about 5-fold, compared to the K_{D} value of the binding of a binding moiety comprising any of the sequences of SEQ ID NOs: 1 to 12 to a CD3-binding domain comprising any of the sequences of SEQ ID NOs: 13 to 17.

In certain embodiments, the amino acid substitution in the binding moiety is a conservative substitution according to Table 1 below.

TABLE 1
Amino Acid Substitutions

| Original | Conservative | |
|---|---|---|
| Residue | Substitutions | Exemplary Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gin |
| Gly (G) | Ala | Ala |
| His (H) | Arg | Asn; Gln; Lys; Arg |
| Ile (I) | Leu | Leu; Val; Met; Ala; Phe; |
| | | Norleucine |
| Leu (L) | Ile | Norleucine; Ile; Val; Met; Ala; |
| | | Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr(Y) | Phe | Trp; Phe; Thr; Ser |
| Val (V) | Leu | Ile; Leu; Met; Phe; Ala; |
| | | Norleucine |

When the binding moiety is an ankyrin repeat domain, in some embodiments, the substitution may be made outside the structural core residues of the ankyrin repeat domain, e.g., in the beta loops that connect the alpha-helices. In other embodiments, the substitution may be made within the structural core residues of the ankyrin repeat domain. For example, the ankyrin domain may comprise the consensus sequence: xDxxGxTPLHLAxxxGxxxJVxVLLxxGADVNA (SEQ ID NO: 68), wherein “x” denotes any amino acid (preferably not cysteine, glycine, or proline); or xDxxGxTPLHLAAxxGHLEIVEVLLKzGADVNA (SEQ ID NO: 69), wherein “x” denotes any amino acid (preferably not cysteine, glycine, or proline), and “z” is selected from the group consisting of asparagine, histidine, or tyrosine. In one embodiment, the substitution is made to residues designated as “x”. In another embodiment, the substitution is made outside the residues designated as “x”.

In addition, the second last position of any ankyrin repeat domain of a binding moiety can be “A” or “L”, and/or the last position can be “A” or “N”. Accordingly, in some embodiments, an ankyrin repeat domain of a binding moiety comprises an amino acid sequence that is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to any one of SEQ ID NOs:1 to 12, and wherein optionally A at the second last position is substituted with L and/or A at the last position is substituted with N. In an exemplary embodiment, an ankyrin repeat domain of a binding moiety comprises an amino acid sequence that is at least about 90% identical to any one of SEQ ID NOs: 1 to 12, and wherein optionally A at the second last position is substituted with L and/or A at the last position is substituted with N. Furthermore, the sequence of any ankyrin repeat domain of a binding moiety may optionally comprise at its N-terminus, a G, an S, or a GS (see below).

In addition, each ankyrin repeat domain of a binding moiety may optionally comprise a “G,” an “S,” or a “GS” sequence at its N-terminus. Accordingly, in some embodiments, an ankyrin repeat domain of a binding moiety comprises an amino acid sequence that is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to any one of SEQ ID NOs: 1 to 12, and further optionally comprises a G, an S, or a GS at its N-terminus. In an exemplary embodiment, an ankyrin repeat domain of a binding moiety comprises an amino acid sequence that is at least about 90% identical to any one of SEQ ID NOs: 1 to 12, and wherein said ankyrin repeat domain further optionally comprises a G, an S, or a GS at its N-terminus. Furthermore, the sequence of any ankyrin repeat domain of a binding moiety may optionally have A at the second last position substituted with L and/or A at the last position substituted with N (see above).

N-Terminal and C-Terminal Capping Sequences

When the binding moieties described herein comprise ankyrin repeat domains, the ankyrin repeat domains may comprise N-terminal or C-terminal capping sequences. Capping sequences refer to additional polypeptide sequences fused to the N- or C-terminal end of ankyrin repeat sequence motif(s) or module(s), wherein said capping sequences form tight tertiary interactions (i.e., tertiary structure interactions) with neighbouring ankyrin repeat sequence motif(s) or module(s) of the ankyrin repeat domains, thereby providing a cap that shields the hydrophobic core of the ankyrin repeat domain at the side from exposure to solvent.

The N- and/or C-terminal capping sequences may be derived from, a capping unit or other structural unit found in a naturally occurring repeat protein adjacent to a repeat unit. Examples of capping sequences are described in International Patent Publication Nos. WO2002/020565 and WO2012/069655, in U.S. Patent Publication No. US 2013/0296221, and by Interlandi et al., J Mal Biol. 2008 Jan. 18; 375(3):837-54. Examples of N-terminal ankyrin capping modules (i.e., N-terminal capping repeats) include SEQ ID NOs: 70 to 73 and examples of C-terminal capping modules (i.e., C-terminal capping repeats) include SEQ ID NOs: 74 to 77.

Drug Molecules

Within the context of the present invention, drug molecules are therapeutic agents that comprise a polypeptide or a protein, wherein said polypeptide or protein contains a site that is capable of being bound by a binding moiety. There is no particular limitation on the drug molecules for use in the present invention, provided that these can be bound by a binding moiety. This means that, for example, the drug molecule may belong to the same “class” as the binding moiety or to a different class as the binding moiety, such that, for example, both the drug molecule and the binding moiety may be antibodies, or both the drug molecule and the binding moiety may be alternative scaffolds (e.g. ankyrin repeat proteins), or the drug molecule may be an antibody and the binding moiety may be an alternative scaffold (e.g. an ankyrin repeat protein), or the drug molecule may be an alternative scaffold (e.g. an ankyrin repeat protein) and the binding moiety may be an antibody. This further means, for example, that the drug molecule itself may comprise different structural moieties, for example, combining an antibody moiety and an alternative scaffold moiety, or combining moieties of different alternative scaffold structures. In case both the drug molecule and the binding moiety are antibodies, it would be clear to the skilled person that the antibodies would be different from each other, including with respect to binding specificity. Similarly, in case both the drug molecule and the binding moiety are alternative scaffolds, it would be clear to the skilled person that the alternative scaffolds would be different from each other, including with respect to binding specificity.

Furthermore, a drug molecule for use in the present invention may contain a half-life extending moiety. A half-life extending moiety extends the serum half-life in vivo of a drug molecule, compared to the same molecule without the half-life extending moiety. Examples of half-life extending moieties include, but are not limited to, polyhistidine, Glu-Glu, glutathione S transferase (GST), thioredoxin, protein A, protein G, an immunoglobulin domain, maltose binding protein (MBP), human serum albumin (HSA) binding domain, or polyethylene glycol (PEG). In some instances, the half-life extending moiety may comprise an ankyrin repeat domain with binding specificity for HSA. In other instances, the half-life extending moiety may comprise an immunoglobulin domain, such as an Fc domain, e.g., the Fc domain of human IgG1, or a variant or derivative thereof.

In some embodiments, drug molecules for use in the present invention comprise alternative scaffolds, wherein the alternative scaffolds are selected from adnectins (monobodies), affibodies, affilins, affimers and aptamers, affitins, alphabodies, anticalins, armadillo repeat protein-based scaffolds, atrimers, avimers, ankyrin repeat protein-based scaffolds (such as DARPin® proteins), fynomers, knottins, and Kunitz domain peptides. Alternative scaffolds are described, e.g., in Yu et al., Annu Rev Anal Chem (Palo Alto Calif). 2017 Jun. 12; 10(1): 293-320. doi:10.1146/annurevanchem-061516-045205.

Drug molecules for use in the present invention also include, but are not limited to, different categories of drugs that are currently approved for clinical use, such as:
(1) immune-checkpoint inhibitors (ICIs);
(2) bispecific antibodies; and
(3) genetically modified immune cells, such as T cells, in particular, chimeric antigen receptor (CAR)-expressing immune cells, such as CAR-T cells.

Drug molecules for use in the present invention also include, but are not limited to, drug molecules that up- or down-regulate the activity of immune checkpoints, herein called “immune checkpoint regulators”. Immune checkpoints are molecules in the immune system that either turn up (co-stimulatory molecules) or turn down (inhibitory molecules) immune signals. In cancer patients, tumors can use these immune checkpoints to protect themselves from immune system attacks, particularly by T cells. Drug molecules used in immune checkpoint therapy can block inhibitory immune checkpoint molecules or activate stimulatory immune checkpoint molecules, thereby restoring immune system function. In recent years, immune checkpoint drugs have become important novel cancer treatment options. Immune checkpoint molecules include, but are not limited to, CD27, CD137, CD137L, 2B4, TIGIT, CD155, ICOS, HVEM, CD40L, LIGHT, TIM-1, OX40, OX40L, DNAM-1, PD-L1, PD1, PD-L2, CTLA-4. CD8, CD40, CEACAMI, CD48, CD70, A2AR, CD39, CD73, B7-H3, B7-H4, BTLA, C5AR1, CCR8, CD226, CD28, CD33, CD38, CD3e, CD47, CD94, ETAR, NKG2A, SIRPα, TLR8, TNFRSF18, IDO1, IDO2, TDO, KIR, LAG-3, TIM-3, TIM-4 and VISTA. In one embodiment, the drug molecule is an immune-checkpoint regulator.

In one embodiment, the drug molecule for use in the present invention comprises an antibody. In another embodiment, the drug molecule comprises a bispecific antibody. In another embodiment, the drug molecule comprises a multispecific antibody. In another embodiment, the drug molecule comprises an antibody that is a T-cell engager drug molecule (TCE).

Bispecific antibodies include TCEs. An example of bispecific antibodies that are TCEs are the molecules known as BiTE™ molecules. These are anti-cancer drugs consisting of two single-chain variable fragments (scFvs) on a single peptide chain. Such TCEs bind to the CD3 (cluster of differentiation 3) molecule on the surface of T cells through one of the scFvs, while the other scFv binds to a tumor-associated antigen (TAA) on the surface of tumor cells. By binding to CD3 and linking a T cell to a tumor cell, the T cell is “activated” and can exert cytotoxic activity on the tumor cell. One example of a bispecific antibody that is a TCE is blinatumomab, which binds to CD3 on the surface of T cells and to CD19 on the surface of B cells. Blinatumomab is approved for use in the treatment of acute lymphoblastic leukaemia.

In one embodiment, the drug molecule for use in the present invention comprises an alternative scaffold. In another embodiment, the drug molecule comprises a bispecific alternative scaffold. In another embodiment, the drug molecule comprises a multispecific alternative scaffold. In another embodiment, the drug molecule comprises an alternative scaffold molecule that is a T-cell engager drug molecule (TCE). In a preferred embodiment, said alternative scaffold is an ankyrin repeat domain. In a further preferred embodiment, the drug molecule for use in the present invention comprises an alternative scaffold, wherein said alternative scaffold is an ankyrin repeat domain having binding specificity for CD3, and wherein said ankyrin repeat domain comprises an amino acid sequence selected from SEQ ID NOs: 13 to 17. In a further preferred embodiment, the drug molecule comprises an ankyrin repeat domain having binding specificity for CD3, wherein said ankyrin repeat domain comprises an amino acid sequence selected from SEQ ID NOs: 13 to 17. In one embodiment, the drug molecule comprises an ankyrin repeat domain having binding specificity for CD3, wherein said ankyrin repeat domain comprises SEQ ID NO: 13. In one embodiment, the drug molecule comprises an ankyrin repeat domain having binding specificity for CD3, wherein said ankyrin repeat domain comprises SEQ ID NO: 14. In one embodiment, the drug molecule comprises an ankyrin repeat domain having binding specificity for CD3, wherein said ankyrin repeat domain comprises SEQ ID NO: 15. In one embodiment, the drug molecule comprises an ankyrin repeat domain having binding specificity for CD3, wherein said ankyrin repeat domain comprises SEQ ID NO: 16. In one embodiment, the drug molecule comprises an ankyrin repeat domain having binding specificity for CD3, wherein said ankyrin repeat domain comprises SEQ ID NO: 17.

Bispecific or multispecific alternative scaffold molecules include TCEs. In one embodiment, the drug molecule is a bispecific or multispecific alternative scaffold molecule, wherein said alternative scaffold molecule is a TCE comprising (i) a CD3-specific binding domain that is an ankyrin repeat domain, and (ii) a TAA-specific binding domain that is an ankyrin repeat domain. Similar to TCEs known as BiTE™, molecules, such TCEs comprising alternative scaffolds are anti-cancer drugs that bind to the CD3 molecule on the surface of T cells through one of the binding domains, while the other binding domain binds to a tumor-associated antigen (TAA) on the surface of tumor cells. By binding to CD3 and linking a T cell to a tumor cell, the T cell is “activated” and can exert cytotoxic activity on the tumor cell.

When the drug molecule is a TCE, the preferred binding site for the binding moiety of the invention is the CD3-specific binding domain of the TCE. By binding to the CD3-specific binding domain of the TCE, the binding moiety blocks the mode of action of the TCE by preventing the TCE from binding to T cells. In one embodiment, the binding of the binding moiety to the CD3-specific binding domain of the TCE is anti-idiotypic.

Numerous bispecific antibodies that are TCEs have been described, including those listed below, with their respective binding targets provided in brackets. For example, as described above, blinatumomab (CD19×CD3; Amgen) binds to the CD3 antigen on a T cell, and to a CD19 antigen on a tumor cell that arose from the B cell lineage. Other bispecifics that are TCEs include, but are not limited to, AMG330 (CD33×CD3; Amgen); flotetuzumab (CD123×CD3; Macrogenics); MCLA117 (Clec12AXCD3; Merus); AMG160 (HLE PSMA×CD3, Amgen); AMG427 (HLE FLT3×CD3, Amgen); AMG562 (HLE CD19×CD3, Amgen); AMG596 (HLE EGFRvIII×CD3, Amgen); AMG673 (HLE CD33×CD3, Amgen); AMG701 (HLE BCMA×CD3, Amgen); AMG757 (HLE DLL3×CD3, Amgen); AMG910 (HLE Claudin18.2×CD3, Amgen); odronextamab (CD20×CD3, Regeneron): mosunetuzumab (CD20×CD3, Roche); glofitamab (CD20×CD3, Roche); and epcoritamab (CD20×CD3. Genmab). Any of such TCEs can be used as a drug molecule in a recombinant binding protein of the invention.

In one embodiment, the drug molecule for use in the present invention comprises an antibody and an alternative scaffold. In another embodiment, the drug molecule comprises two different alternative scaffolds. In another embodiment, the drug molecule comprises a T cell receptor (TCR)-derived antigen-recognition domain.

In one embodiment, the drug molecule for use in the present invention comprises genetically modified immune cells. In a preferred embodiment, said genetically modified immune cells express a chimeric antigen receptor (CAR). In one embodiment, said genetically modified immune cells are genetically modified T cells, such as CAR-expressing T cells (CAR-T cells). In another embodiment, said genetically modified immune cells are genetically modified natural killer (NK) cells, such as CAR-expressing NK cells (CAR-NK cells).

Binding Affinity

The binding moiety of the invention specifically binds to a drug molecule.

In certain embodiments, the binding affinity of the binding moiety to the drug molecule is described in terms of K_{D}. In exemplary embodiments, the K_{D} is about 10⁻⁶ M, about 10⁻⁶ M or less, about 10⁻⁷ M or less, about 10⁻⁸ M or less, about 10⁻⁹ M or less, about 10⁻¹⁰ M or less, or about 10⁻¹¹ M or less, from about 10⁻⁶ M to about 10⁻¹¹ M, from about 10⁻⁶ M to about 10⁻¹⁰ M, from about 10⁻⁶ M to about 10⁻⁹ M, from about 10⁻⁸ M to about 10⁻⁸ M, from about 10⁻⁶ M to about 10⁻⁷ M, from about 10⁻⁷ M to about 10⁻¹¹ M, from about 10⁻⁷ M to about 10⁻¹⁰ M, from about 10⁻⁷ M to about 10⁻⁹ M, from about 10⁻⁷ M to about 10⁻⁸ M, from about 10⁻⁸ M to about 10⁻¹¹ M, from about 10⁻⁸ M to about 10⁻¹⁰ M, from about 10⁻⁸ M to about 10⁻⁹ M, from about 10⁻⁹ M to about 10⁻¹¹ M, or from about 10⁻⁹ M to about 10⁻¹⁰ M, or from about 10⁻¹⁰ M to about 10⁻¹¹ M.

In exemplary embodiments, the binding moiety binds to the drug molecule with a K_{D} value of, or less than: about 1 μM, 750 nm, 500 nm, 250 nm, 100 nM, about 50 nM, about 25 nM, about 10 nM, about 5 nM, about 2 nM, about 1 nM, about 900 μM, about 800 μM, about 700 μM, about B00 μM, about 500 μM, about 400 μM, about 300 μM, about 200 μM, about 100 μM, about 50 μM, about 25 μM or about 10 μM. In one exemplary embodiment, the binding moiety binds to the drug molecule with a K_{D} value of less than or equal to about 1 μM. In another exemplary embodiment, the binding moiety binds to the drug molecule with a K_{D} value of less than or equal to about 500 nM. In another exemplary embodiment, the binding moiety binds to the drug molecule with a K_{D} value of less than or equal to about 100 μM. In yet another exemplary embodiment, the binding moiety binds to the drug molecule with a K_{D} value of less than or equal to about 10 μM. In one embodiment, the binding moiety binds the drug molecule with a dissociation constant (K_{D}) of less than about 1 μM, such as less than about 1 μM, less than about 500 nM, less than about 250 nM, less than about 100 nM or less than about 50 nM. In another embodiment, the binding moiety binds the drug molecule with a dissociation constant (K_{D}) of between about 1 μM and about 10 μM, such as of between about 1 μM and about 10 μM, of between about 1 μM and about 20 μM, of between about 1 μM and about 50 μM, or of between about 1 μM and about 100 μM.

In some embodiments, the binding moiety comprises an ankyrin repeat domain having an amino acid sequence that has at least about 85% sequence identity with SEQ ID NO: 1, and the drug molecule comprises an ankyrin repeat domain having an amino acid sequence that has at least about 85% sequence identity with any of SEQ ID NOs 13 to 17, wherein said binding moiety binds to said drug molecule with a K_{D} value of less than about 1 μM, 750 nm, 500 nm, 250 nm, 100 nM, about 50 nM, about 25 nM, about 10 nM, about 5 nM, about 2 nM, about 1 nM, about 900 μM, about 800 μM, about 700 μM, about 600 μM, about 500 μM, about 400 μM, about 300 μM, about 200 μM, about 100 μM, about 50 μM, about 25 μM or about 10 μM. In some embodiments, the binding moiety comprises an ankyrin repeat domain having an amino acid sequence that has at least about 85% sequence identity with SEQ ID NO: 1, and the drug molecule comprises an ankyrin repeat domain having an amino acid sequence that has at least about 85% sequence identity with SEQ ID Nos: 13 to 17, wherein said binding moiety binds to said drug molecule with a K_{D} value in the range of about 1 μM to about 10 μM.

In some embodiments, the binding moiety comprises an ankyrin repeat domain having an amino acid sequence that has at least about 85% sequence identity with SEQ ID NO: 2, and the drug molecule comprises an ankyrin repeat domain having an amino acid sequence that has at least about 85% sequence identity with any of SEQ ID NOs 13 to 17, wherein said binding moiety binds to said drug molecule with a K_{D} value of less than about 1 μM, 750 nm, 500 nm, 250 nm, 100 nM, about 50 nM, about 25 nM, about 10 nM, about 5 nM, about 2 nM, about 1 nM, about 900 pM, about 800 pM, about 700 pM, about 600 pM, about 500 pM, about 400 pM, about 300 pM, about 200 pM, about 100 pM, about 50 pM, about 25 pM or about 10 pM. In some embodiments, the binding moiety comprises an ankyrin repeat domain having an amino acid sequence that has at least about 85% sequence identity with SEQ ID NO: 2, and the drug molecule comprises an ankyrin repeat domain having an amino acid sequence that has at least about 85% sequence identity with SEQ ID NOs 13 to 17, wherein said binding moiety binds to said drug molecule with a K_{D} value in the range of about 1 pM to about 10 pM.

In some embodiments, the binding moiety comprises an ankyrin repeat domain having an amino acid sequence that has at least about 85% sequence identity with SEQ ID NO: 3, and the drug molecule comprises an ankyrin repeat domain having an amino acid sequence that has at least about 85% sequence identity with any of SEQ ID NOs 13 to 17, wherein said binding moiety binds to said drug molecule with a K_{D} value of less than about 1 μM, 750 nm, 500 nm, 250 nm, 100 nM, about 50 nM, about 25 nM, about 10 nM, about 5 nM, about 2 nM, about 1 nM, about 900 pM, about 800 pM, about 700 pM, about 600 pM, about 500 pM, about 400 pM, about 300 pM, about 200 pM, about 100 pM, about 50 pM, about 25 pM or about 10 pM. In some embodiments, the binding moiety comprises an ankyrin repeat domain having an amino acid sequence that has at least about 85% sequence identity with SEQ ID NO: 3, and the drug molecule comprises an ankyrin repeat domain having an amino acid sequence that has at least about 85% sequence identity with SEQ ID NOs 13 to 17, wherein said binding moiety binds to said drug molecule with a K_{D} value in the range of about 1 μM to about 10 μM.

In some embodiments, the binding moiety comprises an ankyrin repeat domain having an amino acid sequence that has at least about 85% sequence identity with SEQ ID NO: 4, and the drug molecule comprises an ankyrin repeat domain having an amino acid sequence that has at least about 85% sequence identity with any of SEQ ID NOs 13 to 17, wherein said binding moiety binds to said drug molecule with a K_{D} value of less than about 1 μM, 750 nm, 500 nm, 250 nm, 100 nM, about 50 nM, about 25 nM, about 10 nM, about 5 nM, about 2 nM, about 1 nM, about 900 μM, about 800 μM, about 700 μM, about 600 μM, about 500 μM, about 400 μM, about 300 μM, about 200 μM, about 100 μM, about 50 μM, about 25 μM or about 10 μM. In some embodiments, the binding moiety comprises an ankyrin repeat domain having an amino acid sequence that has at least about 85% sequence identity with SEQ ID NO: 4, and the drug molecule comprises an ankyrin repeat domain having an amino acid sequence that has at least about 85% sequence identity with SEQ ID NOs 13 to 17, wherein said binding moiety binds to said drug molecule with a K_{D} value in the range of about 1 μM to about 10 μM.

In some embodiments, the binding moiety comprises an ankyrin repeat domain having an amino acid sequence that has at least about 85% sequence identity with SEQ ID NO: 5, and the drug molecule comprises an ankyrin repeat domain having an amino acid sequence that has at least about 85% sequence identity with any of SEQ ID NOs 13 to 17, wherein said binding moiety binds to said drug molecule with a K_{D} value of less than about 1 μM, 750 nm, 500 nm, 250 nm, 100 nM, about 50 nM, about 25 nM, about 10 nM, about 5 nM, about 2 nM, about 1 nM, about 900 pM, about 800 pM, about 700 pM, about 600 pM, about 500 pM, about 400 pM, about 300 pM, about 200 pM, about 100 pM, about 50 pM, about 25 pM or about 10 pM. In some embodiments, the binding moiety comprises an ankyrin repeat domain having an amino acid sequence that has at least about 85% sequence identity with SEQ ID NO: 5, and the drug molecule comprises an ankyrin repeat domain having an amino acid sequence that has at least about 85% sequence identity with SEQ ID NOs 13 to 17, wherein said binding moiety binds to said drug molecule with a K_{D} value in the range of about 1 μM to about 10 pM.

In some embodiments, the binding moiety comprises an ankyrin repeat domain having an amino acid sequence that has at least about 85% sequence identity with SEQ ID NO: 6, and the drug molecule comprises an ankyrin repeat domain having an amino acid sequence that has at least about 85% sequence identity with any of SEQ ID NOs 13 to 17, wherein said binding moiety binds to said drug molecule with a K_{D} value of less than about 1 μM, 750 nm, 500 nm, 250 nm, 100 nM, about 50 nM, about 25 nM, about 10 nM, about 5 nM, about 2 nM, about 1 nM, about 900 μM, about 800 pM, about 700 pM, about 600 pM, about 500 pM, about 400 pM, about 300 pM, about 200 pM, about 100 pM, about 50 pM, about 25 pM or about 10 pM. In some embodiments, the binding moiety comprises an ankyrin repeat domain having an amino acid sequence that has at least about 85% sequence identity with SEQ ID NO: 6, and the drug molecule comprises an ankyrin repeat domain having an amino acid sequence that has at least about 85% sequence identity with SEQ ID NOs 13 to 17, wherein said binding moiety binds to said drug molecule with a K_{D} value in the range of about 1 μM to about 10 pM.

In some embodiments, the binding moiety comprises an ankyrin repeat domain having an amino acid sequence that has at least about 85% sequence identity with SEQ ID NO: 7, and the drug molecule comprises an ankyrin repeat domain having an amino acid sequence that has at least about 85% sequence identity with any of SEQ ID NOs 13 to 17, wherein said binding moiety binds to said drug molecule with a K_{D} value of less than about 1 μM, 750 nm, 500 nm, 250 nm, 100 nM, about 50 nM, about 25 nM, about 10 nM, about 5 nM, about 2 nM, about 1 nM, about 900 μM, about 800 pM, about 700 pM, about 600 pM, about 500 pM, about 400 pM, about 300 pM, about 200 pM, about 100 pM, about 50 pM, about 25 pM or about 10 pM. In some embodiments, the binding moiety comprises an ankyrin repeat domain having an amino acid sequence that has at least about 85% sequence identity with SEQ ID NO: 7, and the drug molecule comprises an ankyrin repeat domain having an amino acid sequence that has at least about 85% sequence identity with SEQ ID NOs 13 to 17, wherein said binding moiety binds to said drug molecule with a K_{D} value in the range of about 1 μM to about 10 pM.

In some embodiments, the binding moiety comprises an ankyrin repeat domain having an amino acid sequence that has at least about 85% sequence identity with SEQ ID NO: 8, and the drug molecule comprises an ankyrin repeat domain having an amino acid sequence that has at least about 85% sequence identity with any of SEQ ID NOs 13 to 17, wherein said binding moiety binds to said drug molecule with a K_{D} value of less than about 1 μM, 750 nm, 500 nm, 250 nm, 100 nM, about 50 nM, about 25 nM, about 10 nM, about 5 nM, about 2 nM, about 1 nM, about 900 pM, about 800 pM, about 700 pM, about 600 pM, about 500 pM, about 400 pM, about 300 pM, about 200 pM, about 100 pM, about 50 pM, about 25 pM or about 10 pM. In some embodiments, the binding moiety comprises an ankyrin repeat domain having an amino acid sequence that has at least about 85% sequence identity with SEQ ID NO: 8, and the drug molecule comprises an ankyrin repeat domain having an amino acid sequence that has at least about 85% sequence identity with SEQ ID NOs 13 to 17, wherein said binding moiety binds to said drug molecule with a K_{D} value in the range of about 1 μM to about 10 pM.

In some embodiments, the binding moiety comprises an ankyrin repeat domain having an amino acid sequence that has at least about 85% sequence identity with SEQ ID NO: 9, and the drug molecule comprises an ankyrin repeat domain having an amino acid sequence that has at least about 85% sequence identity with any of SEQ ID NOs 13 to 17, wherein said binding moiety binds to said drug molecule with a K_{D} value of less than about 1 μM, 750 nm, 500 nm, 250 nm, 100 nM, about 50 nM, about 25 nM, about 10 nM, about 5 nM, about 2 nM, about 1 nM, about 900 pM, about 800 pM, about 700 pM, about 600 pM, about 500 pM, about 400 pM, about 300 pM, about 200 pM, about 100 pM, about 50 pM, about 25 pM or about 10 pM. In some embodiments, the binding moiety comprises an ankyrin repeat domain having an amino acid sequence that has at least about 85% sequence identity with SEQ ID NO: 9, and the drug molecule comprises an ankyrin repeat domain having an amino acid sequence that has at least about 85% sequence identity with SEQ ID NOs 13 to 17, wherein said binding moiety binds to said drug molecule with a K_{D} value in the range of about 1 μM to about 10 pM.

In some embodiments, the binding moiety comprises an ankyrin repeat domain having an amino acid sequence that has at least about 85% sequence identity with SEQ ID NO: 10, and the drug molecule comprises an ankyrin repeat domain having an amino acid sequence that has at least about 85% sequence identity with any of SEQ ID NOs 13 to 17, wherein said binding moiety binds to said drug molecule with a K_{D} value of less than about 1 μM, 750 nm, 500 nm, 250 nm, 100 nM, about 50 nM, about 25 nM, about 10 nM, about 5 nM, about 2 nM, about 1 nM, about 900 pM, about 800 pM, about 700 pM, about 600 pM, about 500 pM, about 400 pM, about 300 pM, about 200 pM, about 100 pM, about 50 pM, about 25 pM or about 10 pM. In some embodiments, the binding moiety comprises an ankyrin repeat domain having an amino acid sequence that has at least about 85% sequence identity with SEQ ID NO: 10, and the drug molecule comprises an ankyrin repeat domain having an amino acid sequence that has at least about 85% sequence identity with SEQ ID NOs 13 to 17, wherein said binding moiety binds to said drug molecule with a K_{D} value in the range of about 1 μM to about 10 pM.

In some embodiments, the binding moiety comprises an ankyrin repeat domain having an amino acid sequence that has at least about 85% sequence identity with SEQ ID NO: 11 and the drug molecule comprises an ankyrin repeat domain having an amino acid sequence that has at least about 85% sequence identity with any of SEQ ID NOs 13 to 17, wherein said binding moiety binds to said drug molecule with a K_{D} value of less than about 1 μM, 750 nm, 500 nm, 250 nm, 100 nM, about 50 nM, about 25 nM, about 10 nM, about 5 nM, about 2 nM, about 1 nM, about 900 pM, about 800 pM, about 700 pM, about 600 pM, about 500 pM, about 400 pM, about 300 pM, about 200 pM, about 100 pM, about 50 pM, about 25 pM or about 10 pM. In some embodiments, the binding moiety comprises an ankyrin repeat domain having an amino acid sequence that has at least about 85% sequence identity with SEQ ID NO: 11, and the drug molecule comprises an ankyrin repeat domain having an amino acid sequence that has at least about 85% sequence identity with SEQ ID NOs 13 to 17, wherein said binding moiety binds to said drug molecule with a K_{D} value in the range of about 1 μM to about 10 pM.

In some embodiments, the binding moiety comprises an ankyrin repeat domain having an amino acid sequence that has at least about 85% sequence identity with SEQ ID NO: 12, and the drug molecule comprises an ankyrin repeat domain having an amino acid sequence that has at least about 85% sequence identity with any of SEQ ID NOs 13 to 17, wherein said binding moiety binds to said drug molecule with a K_{D} value of less than about 1 μM, 750 nm, 500 nm, 250 nm, 100 nM, about 50 nM, about 25 nM, about 10 nM, about 5 nM, about 2 nM, about 1 nM, about 900 pM, about 800 pM, about 700 pM, about 600 pM, about 500 pM, about 400 pM, about 300 pM, about 200 pM, about 100 pM, about 50 pM, about 25 pM or about 10 pM. In some embodiments, the binding moiety comprises an ankyrin repeat domain having an amino acid sequence that has at least about 85% sequence identity with SEQ ID NO: 12, and the drug molecule comprises an ankyrin repeat domain having an amino acid sequence that has at least about 85% sequence identity with SEQ ID NOs 13 to 17, wherein said binding moiety binds to said drug molecule with a K_{D} value in the range of about 1 μM to about 10 pM.

When the binding moiety is bound to the drug molecule, the drug molecule is unable to exert a biological activity, such as, for example, binding to a biological target molecule. Upon proteolytic cleavage of the peptide linker connecting the binding moiety and the drug molecule in a recombinant protein of the invention, the active drug is released into the subject.

Recombinant Binding Proteins

The recombinant binding proteins of the present invention comprise (i) a binding moiety as defined herein and (ii) a drug molecule as defined herein. Said binding moiety reversibly binds to said drug molecule, and the action of binding inhibits a biological activity of said drug molecule. As described herein, the binding moiety and drug molecule are connected by a peptide linker which comprises a protease cleavage site.

A “linker” or “linking moiety” is a molecule or group of molecules that connects two separate entities. Two types of linkers are encompassed by the present invention: protease-cleavable linkers and non-protease-cleavable linkers. In recombinant proteins of the invention, the linker between the binding moiety and drug molecule should be a protease-cleavable linker to allow the drug molecule to be “released” from binding with the binding moiety. However, non-protease-cleavable linkers may be present in the prodrug molecule, such as between the binding molecule and a half-life extending moiety, and/or between different domains of the drug molecule, such as a binding domain with specificity for CD3 and a binding domain with specificity for a tumor-associated antigen (TAA). Examples of these different types of linker are shown in FIG. 1. A protease cleavable linker is shown in FIG. 1 between the Binder and the CD3 binding moiety. As illustrated in FIG. 1, the linker between α-CD3 and Binder is composed of a peptide linker that is cleavable by proteases in the tumor microenvironment. The prodrug CD3-PDD is inactive upon injection into circulation, as the binding to T-cells via its α-CD3 arm is inhibited by the covalently linked Binder. Once entering the tumor microenvironment (TME), the peptide linker between α-CD3 and Binder is cut by tumor-associated proteases, and the drug molecule can then exert its biological activity by binding to TAA on tumor cells via its α-TAA arm and to CD3 on T-cells via its α-CD3 arm, leading to T-cell mediated tumor cell killing.

In some embodiments, the protease-cleavable linker has an amino acid sequence that has at least about 80%, at least about 81%, at least about 82%, at least about 83%, at least about 84%, at least about 85%, at least about 86%, at least about 87%, at least about 88%, at least about 89%, at least about 90%, at least about 91%, about 92%, about 93%, about 94%, about 95%, about 96%, about 97%, about 98%, about 99%, or 100% sequence identity with an amino acid sequence selected from the group consisting of SEQ ID NOs 18 to 20.

Non-protease-cleavable linkers may include a covalent linker, for example, a disulfide bond, a polypeptide bond or a crosslinking agent; or a non-covalent linker, to produce a heterodimeric protein. Non-protease-cleavable linkers may be present, for example, between the binding moiety and a half-life extending moiety.

In some embodiments, the non-protease-cleavable inker is a peptide linker. In some embodiments, the peptide linker comprises about 1 to 50 amino acid residues. Exemplary linkers includes, e.g., a glycine rich peptide; a peptide comprising glycine and serine; a peptide having a sequence [Gly-Gly-Ser]ₙ, wherein n is 1, 2, 3, 4, 5, or 6; or a peptide having a sequence [Gly-Gly-Gly-Gly-Ser]ₙ (SEQ ID NO: 83), wherein n is 1, 2, 3, 4, 5, or 6. A glycine rich peptide linker comprises a peptide linker, wherein at least 25% of the residues are glycine. Glycine rich peptide linkers are well known in the art (e.g., Chichili et al. Protein Sci. 2013 February; 22(2): 153-167).

In some embodiments, the peptide linker is a proline-threonine rich peptide linker. In one embodiment, the linker is a proline-threonine rich peptide linker of any one of SEQ ID NOs: 78 to 82. In an exemplary embodiment, the linker is the proline-threonine rich peptide linker of SEQ ID NO: 81. In another exemplary embodiment, the linker is the proline-threonine rich peptide linker of SEQ ID NO: 82.

In the recombinant binding proteins of the present invention, any binding moiety listed above may be combined with any drug molecule listed above, provided that the binding moiety has the desired binding affinity and specificity for the drug molecule. In particular, any binding moiety and drug molecule described above, in particular any of the specifically disclosed combinations with a binding coefficient (such as K_{D}) explicitly disclosed are encompassed by the present invention,

In one embodiment, the recombinant binding protein comprises (i) a binding moiety comprising an ankyrin repeat domain having an amino acid sequence selected from the group consisting of (1) SEQ ID NO: 1 and (2) sequences that have at least about 85% amino acid sequence identity with SEQ ID NO: 1; and (i) a drug molecule comprising an ankyrin repeat domain having an amino acid sequence selected from the group consisting of (a) SEQ ID NOs: 13 to 17 and (2) sequences that have at least about 85% amino acid sequence identity with SEQ ID NOs: 13 to 17.

In one embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 1, and (2) a drug molecule comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with any of SEQ ID NOs: 13 to 17.

In one embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 1, and (2) a drug molecule comprising an ankyrin repeat domain having at least about 90% amino acid sequence identity with any of SEQ ID NOs: 13 to 17. In another embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 1, and (2) a drug molecule comprising any of SEQ ID NOs: 13 to 17.

In another embodiment, the recombinant binding protein comprises (1) a binding moiety comprising an ankyrin repeat domain having the amino acid sequence of SEQ ID NO: 1, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 1 have been substituted by other amino acids, and (2) a drug molecule comprising an ankyrin repeat domain having an amino acid sequence selected from the group consisting of (a) SEQ ID NOs: 13 to 17 and (2) sequences that have at least about 85% amino acid sequence identity with SEQ ID NOs: 13 to 17.

In another embodiment, the recombinant binding protein comprises (1) a binding moiety comprising an ankyrin repeat domain having the amino acid sequence of SEQ ID NO: 1, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 1 have been substituted by other amino acids, and (2) a drug molecule comprising an ankyrin repeat domain having at least about 90% amino acid sequence identity with any of SEQ ID NOs: 13 to 17. In another embodiment, the recombinant binding protein comprises (1) a binding moiety comprising an ankyrin repeat domain having the amino acid sequence of SEQ ID NO: 1, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 1 have been substituted by other amino acids, and (2) a drug molecule comprising any of SEQ ID NOs: 13 to 17.

In another embodiment, the recombinant binding protein comprises (1) a binding moiety comprising an ankyrin repeat domain having SEQ ID NO: 1, and (2) a drug molecule comprising an ankyrin repeat domain comprising any of SEQ ID NOs: 13 to 17. In another embodiment, the recombinant binding protein comprises (1) a binding moiety comprising an ankyrin repeat domain having SEQ ID NO: 1, and (2) a drug molecule comprising an ankyrin repeat domain comprising any of SEQ ID NOs: 15 to 16.

In one embodiment, the recombinant binding protein comprises (i) a binding moiety comprising an ankyrin repeat domain having an amino acid sequence selected from the group consisting of (1) SEQ ID NO: 2 and (2) sequences that have at least about 85% amino acid sequence identity with SEQ ID NO: 2; and (ii) a drug molecule comprising an ankyrin repeat domain having an amino acid sequence selected from the group consisting of (a) SEQ ID NOs: 13 to 17 and (2) sequences that have at least about 85% amino acid sequence identity with SEQ ID NOs: 13 to 17.

In one embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 2, and (2) a drug molecule comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with any of SEQ ID NOs: 13 to 17.

In one embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 2, and (2) a drug molecule comprising an ankyrin repeat domain having at least about 90% amino acid sequence identity with any of SEQ ID NOs: 13 to 17. In another embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 2, and (2) a drug molecule comprising any of SEQ ID NOs: 13 to 17.

In another embodiment, the recombinant binding protein comprises (1) a binding moiety comprising an ankyrin repeat domain having the amino acid sequence of SEQ ID NO: 2, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 2 have been substituted by other amino acids, and (2) a drug molecule comprising an ankyrin repeat domain having an amino acid sequence selected from the group consisting of (a) SEQ ID NOs: 13 to 17 and (2) sequences that have at least about 85% amino acid sequence identity with SEQ ID NOs: 13 to 17.

In another embodiment, the recombinant binding protein comprises (1) a binding moiety comprising an ankyrin repeat domain having the amino acid sequence of SEQ ID NO: 2, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 2 have been substituted by other amino acids, and (2) a drug molecule comprising an ankyrin repeat domain having at least about 90% amino acid sequence identity with any of SEQ ID NOs: 13 to 17. In another embodiment, the recombinant binding protein comprises (1) a binding moiety comprising an ankyrin repeat domain having the amino acid sequence of SEQ ID NO: 2, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 2 have been substituted by other amino acids, and (2) a drug molecule comprising any of SEQ ID NOs: 13 to 17.

In another embodiment, the recombinant binding protein comprises (1) a binding moiety comprising an ankyrin repeat domain having SEQ ID NO: 2, and (2) a drug molecule comprising an ankyrin repeat domain comprising any of SEQ ID NOs: 13 to 17. In another embodiment, the recombinant binding protein comprises (1) a binding moiety comprising an ankyrin repeat domain having SEQ ID NO: 2, and (2) a drug molecule comprising an ankyrin repeat domain comprising any of SEQ ID NOs: 15 to 16.

In one embodiment, the recombinant binding protein comprises (i) a binding moiety comprising an ankyrin repeat domain having an amino acid sequence selected from the group consisting of (1) SEQ ID NO: 3 and (2) sequences that have at least about 85% amino acid sequence identity with SEQ ID NO: 3; and (ii) a drug molecule comprising an ankyrin repeat domain having an amino acid sequence selected from the group consisting of (a) SEQ ID NOs: 13 to 17 and (2) sequences that have at least about 85% amino acid sequence identity with SEQ ID NOs: 13 to 17.

In one embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 3, and (2) a drug molecule comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with any of SEQ ID NOs: 13 to 17.

In one embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 3, and (2) a drug molecule comprising an ankyrin repeat domain having at least about 90% amino acid sequence identity with any of SEQ ID NOs: 13 to 17. In another embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 3, and (2) a drug molecule comprising any of SEQ ID NOs: 13 to 17.

In another embodiment, the recombinant binding protein comprises (1) a binding moiety comprising an ankyrin repeat domain having the amino acid sequence of SEQ ID NO: 3, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 3 have been substituted by other amino acids, and (2) a drug molecule comprising an ankyrin repeat domain having an amino acid sequence selected from the group consisting of (a) SEQ ID NOs: 13 to 17 and (2) sequences that have at least about 85% amino acid sequence identity with SEQ ID NOs: 13 to 17.

In another embodiment, the recombinant binding protein comprises (1) a binding moiety comprising an ankyrin repeat domain having the amino acid sequence of SEQ ID NO: 3, wherein optionally up to 15, up to 14, up to 13, up to 12, up to 11, up to 10, up to 9, up to B, up to 7, up to 6, up to 5, up to 4, up to 3, up to 2, or up to 1 amino acids in SEQ 1D NO: 3 have been substituted by other amino acids, and (2) a drug molecule comprising an ankyrin repeat domain having at least about 90% amino acid sequence identity with any of SEQ ID NOs: 13 to 17. In another embodiment, the recombinant binding protein comprises (1) a binding moiety comprising an ankyrin repeat domain having the amino acid sequence of SEQ ID NO: 3, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 3 have been substituted by other amino acids, and (2) a drug molecule comprising any of SEQ ID NOs: 13 to 17.

In another embodiment, the recombinant binding protein comprises (1) a binding moiety comprising an ankyrin repeat domain having SEQ ID NO: 3, and (2) a drug molecule comprising an ankyrin repeat domain comprising any of SEQ ID NOs: 13 to 17. In another embodiment, the recombinant binding protein comprises (1) a binding moiety comprising an ankyrin repeat domain having SEQ ID NO: 3, and (2) a drug molecule comprising an ankyrin repeat domain comprising any of SEQ ID NOs: 15 to 16.

In one embodiment, the recombinant binding protein comprises (i) a binding moiety comprising an ankyrin repeat domain having an amino acid sequence selected from the group consisting of (1) SEQ ID NO: 4 and (2) sequences that have at least about 85% amino acid sequence identity with SEQ ID NO: 4; and (i) a drug molecule comprising an ankyrin repeat domain having an amino acid sequence selected from the group consisting of (a) SEQ ID NOs: 13 to 17 and (2) sequences that have at least about 85% amino acid sequence identity with SEQ ID NOs: 13 to 17.

In one embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 4, and (2) a drug molecule comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with any of SEQ ID NOs: 13 to 17.

In one embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 4, and (2) a drug molecule comprising an ankyrin repeat domain having at least about 90% amino acid sequence identity with any of SEQ ID NOs: 13 to 17. In another embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 4, and (2) a drug molecule comprising any of SEQ ID NOs: 13 to 17.

In another embodiment, the recombinant binding protein comprises (1) a binding moiety comprising an ankyrin repeat domain having the amino acid sequence of SEQ ID NO: 4, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 4 have been substituted by other amino acids, and (2) a drug molecule comprising an ankyrin repeat domain having an amino acid sequence selected from the group consisting of (a) SEQ ID NOs: 13 to 17 and (2) sequences that have at least about 85% amino acid sequence identity with SEQ ID NOs: 13 to 17.

In another embodiment, the recombinant binding protein comprises (1) a binding moiety comprising an ankyrin repeat domain having the amino acid sequence of SEQ ID NO: 4, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 4 have been substituted by other amino acids, and (2) a drug molecule comprising an ankyrin repeat domain having at least about 90% amino acid sequence identity with any of SEQ ID NOs: 13 to 17. In another embodiment, the recombinant binding protein comprises (1) a binding moiety comprising an ankyrin repeat domain having the amino acid sequence of SEQ ID NO: 4, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 4 have been substituted by other amino acids, and (2) a drug molecule comprising any of SEQ ID NOs: 13 to 17.

In another embodiment, the recombinant binding protein comprises (1) a binding moiety comprising an ankyrin repeat domain having SEQ ID NO: 4, and (2) a drug molecule comprising an ankyrin repeat domain comprising any of SEQ ID NOs: 13 to 17. In another embodiment, the recombinant binding protein comprises (1) a binding moiety comprising an ankyrin repeat domain having SEQ ID NO: 4, and (2) a drug molecule comprising an ankyrin repeat domain comprising any of SEQ ID NOs: 15 to 16.

In one embodiment, the recombinant binding protein comprises (i) a binding moiety comprising an ankyrin repeat domain having an amino acid sequence selected from the group consisting of (1) SEQ ID NO: 5 and (2) sequences that have at least about 85% amino acid sequence identity with SEQ ID NO: 5; and (ii) a drug molecule comprising an ankyrin repeat domain having an amino acid sequence selected from the group consisting of (a) SEQ ID NOs: 13 to 17 and (2) sequences that have at least about 85% amino acid sequence identity with SEQ ID NOs: 13 to 17.

In one embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 5, and (2) a drug molecule comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with any of SEQ ID NOs: 13 to 17.

In one embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 5, and (2) a drug molecule comprising an ankyrin repeat domain having at least about 90% amino acid sequence identity with any of SEQ ID NOs: 13 to 17. In another embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 5, and (2) a drug molecule comprising any of SEQ ID NOs: 13 to 17.

In another embodiment, the recombinant binding protein comprises (1) a binding moiety comprising an ankyrin repeat domain having the amino acid sequence of SEQ ID NO: 5, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 5 have been substituted by other amino acids, and (2) a drug molecule comprising an ankyrin repeat domain having an amino acid sequence selected from the group consisting of (a) SEQ ID NOs: 13 to 17 and (2) sequences that have at least about 85% amino acid sequence identity with SEQ ID NOs: 13 to 17.

In another embodiment, the recombinant binding protein comprises (1) a binding moiety comprising an ankyrin repeat domain having the amino acid sequence of SEQ ID NO: 5, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 5 have been substituted by other amino acids, and (2) a drug molecule comprising an ankyrin repeat domain having at least about 90% amino acid sequence identity with any of SEQ ID NOs: 13 to 17. In another embodiment, the recombinant binding protein comprises (1) a binding moiety comprising an ankyrin repeat domain having the amino acid sequence of SEQ ID NO: 5, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 5 have been substituted by other amino acids, and (2) a drug molecule comprising any of SEQ ID NOs: 13 to 17.

In another embodiment, the recombinant binding protein comprises (1) a binding moiety comprising an ankyrin repeat domain having SEQ ID NO: 5, and (2) a drug molecule comprising an ankyrin repeat domain comprising any of SEQ ID NOs: 13 to 17. In another embodiment, the recombinant binding protein comprises (1) a binding moiety comprising an ankyrin repeat domain having SEQ ID NO: 5, and (2) a drug molecule comprising an ankyrin repeat domain comprising any of SEQ ID NO&: 15 to 16.

In one embodiment, the recombinant binding protein comprises (i) a binding moiety comprising an ankyrin repeat domain having an amino acid sequence selected from the group consisting of (1) SEQ ID NO: 6 and (2) sequences that have at least about 85% amino acid sequence identity with SEQ ID NO: 6; and (i) a drug molecule comprising an ankyrin repeat domain having an amino acid sequence selected from the group consisting of (a) SEQ ID NOs: 13 to 17 and (2) sequences that have at least about 85% amino acid sequence identity with SEQ ID NOs: 13 to 17.

In one embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 6, and (2) a drug molecule comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with any of SEQ ID NOs: 13 to 17.

In one embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 6, and (2) a drug molecule comprising an ankyrin repeat domain having at least about 90% amino acid sequence identity with any of SEQ ID NOs: 13 to 17. In another embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 6, and (2) a drug molecule comprising any of SEQ ID NOs: 13 to 17.

In another embodiment, the recombinant binding protein comprises (1) a binding moiety comprising an ankyrin repeat domain having the amino acid sequence of SEQ ID NO: 6, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 6 have been substituted by other amino acids, and (2) a drug molecule comprising an ankyrin repeat domain having an amino acid sequence selected from the group consisting of (a) SEQ ID NOs: 13 to 17 and (2) sequences that have at least about 85% amino acid sequence identity with SEQ ID NOs: 13 to 17.

In another embodiment, the recombinant binding protein comprises (1) a binding moiety comprising an ankyrin repeat domain having the amino acid sequence of SEQ ID NO: 6, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 6 have been substituted by other amino acids, and (2) a drug molecule comprising an ankyrin repeat domain having at least about 90% amino acid sequence identity with any of SEQ ID NOs: 13 to 17. In another embodiment, the recombinant binding protein comprises (1) a binding moiety comprising an ankyrin repeat domain having the amino acid sequence of SEQ ID NO: 6, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 6 have been substituted by other amino acids, and (2) a drug molecule comprising any of SEQ ID NOs: 13 to 17.

In another embodiment, the recombinant binding protein comprises (1) a binding moiety comprising an ankyrin repeat domain having SEQ ID NO: 6, and (2) a drug molecule comprising an ankyrin repeat domain comprising any of SEQ ID NOs: 13 to 17. In another embodiment, the recombinant binding protein comprises (1) a binding moiety comprising an ankyrin repeat domain having SEQ ID NO: 6, and (2) a drug molecule comprising an ankyrin repeat domain comprising any of SEQ ID NOs: 15 to 16.

In one embodiment, the recombinant binding protein comprises (i) a binding moiety comprising an ankyrin repeat domain having an amino acid sequence selected from the group consisting of (1) SEQ ID NO: 7 and (2) sequences that have at least about 85% amino acid sequence identity with SEQ ID NO: 7; and (i) a drug molecule comprising an ankyrin repeat domain having an amino acid sequence selected from the group consisting of (a) SEQ ID NOs: 13 to 17 and (2) sequences that have at least about 85% amino acid sequence identity with SEQ ID NOs: 13 to 17.

In one embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 7, and (2) a drug molecule comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with any of SEQ ID NOs: 13 to 17.

In one embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 7, and (2) a drug molecule comprising an ankyrin repeat domain having at least about 90% amino acid sequence identity with any of SEQ ID NOs: 13 to 17. In another embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 7, and (2) a drug molecule comprising any of SEQ ID NOs: 13 to 17.

In another embodiment, the recombinant binding protein comprises (1) a binding moiety comprising an ankyrin repeat domain having the amino acid sequence of SEQ ID NO: 7, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 7 have been substituted by other amino acids, and (2) a drug molecule comprising an ankyrin repeat domain having an amino acid sequence selected from the group consisting of (a) SEQ ID NOs: 13 to 17 and (2) sequences that have at least about 85% amino acid sequence identity with SEQ ID NOs: 13 to 17.

In another embodiment, the recombinant binding protein comprises (1) a binding moiety comprising an ankyrin repeat domain having the amino acid sequence of SEQ ID NO: 7, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 7 have been substituted by other amino acids, and (2) a drug molecule comprising an ankyrin repeat domain having at least about 90% amino acid sequence identity with any of SEQ ID NOs: 13 to 17. In another embodiment, the recombinant binding protein comprises (1) a binding moiety comprising an ankyrin repeat domain having the amino acid sequence of SEQ ID NO: 7, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 7 have been substituted by other amino acids, and (2) a drug molecule comprising any of SEQ ID NOs: 13 to 17.

In another embodiment, the recombinant binding protein comprises (1) a binding moiety comprising an ankyrin repeat domain having SEQ ID NO: 7, and (2) a drug molecule comprising an ankyrin repeat domain comprising any of SEQ ID NOs: 13 to 17. In another embodiment, the recombinant binding protein comprises (1) a binding moiety comprising an ankyrin repeat domain having SEQ ID NO: 7, and (2) a drug molecule comprising an ankyrin repeat domain comprising any of SEQ ID NOs: 15 to 16.

In one embodiment, the recombinant binding protein comprises (i) a binding moiety comprising an ankyrin repeat domain having an amino acid sequence selected from the group consisting of (1) SEQ ID NO: 8 and (2) sequences that have at least about 85% amino acid sequence identity with SEQ ID NO: 8; and (ii) a drug molecule comprising an ankyrin repeat domain having an amino acid sequence selected from the group consisting of (a) SEQ ID NOs: 13 to 17 and (2) sequences that have at least about 85% amino acid sequence identity with SEQ ID NOs: 13 to 17.

In one embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 8, and (2) a drug molecule comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with any of SEQ ID NOs: 13 to 17.

In one embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 8, and (2) a drug molecule comprising an ankyrin repeat domain having at least about 90% amino acid sequence identity with any of SEQ ID NOs: 13 to 17. In another embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 8, and (2) a drug molecule comprising any of SEQ ID NOs: 13 to 17.

In another embodiment, the recombinant binding protein comprises (1) a binding moiety comprising an ankyrin repeat domain having the amino acid sequence of SEQ ID NO: 8, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 8 have been substituted by other amino acids, and (2) a drug molecule comprising an ankyrin repeat domain having an amino acid sequence selected from the group consisting of (a) SEQ ID NOs: 13 to 17 and (2) sequences that have at least about 85% amino acid sequence identity with SEQ ID NOs: 13 to 17.

In another embodiment, the recombinant binding protein comprises (1) a binding moiety comprising an ankyrin repeat domain having the amino acid sequence of SEQ ID NO: 8, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 8 have been substituted by other amino acids, and (2) a drug molecule comprising an ankyrin repeat domain having at least about 90% amino acid sequence identity with any of SEQ ID NOs: 13 to 17. In another embodiment, the recombinant binding protein comprises (1) a binding moiety comprising an ankyrin repeat domain having the amino acid sequence of SEQ ID NO: 8, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 8 have been substituted by other amino acids, and (2) a drug molecule comprising any of SEQ ID NOs: 13 to 17.

In another embodiment, the recombinant binding protein comprises (1) a binding moiety comprising an ankyrin repeat domain having SEQ ID NO: 8, and (2) a drug molecule comprising an ankyrin repeat domain comprising any of SEQ ID NOs: 13 to 17. In another embodiment, the recombinant binding protein comprises (1) a binding moiety comprising an ankyrin repeat domain having SEQ ID NO: 8, and (2) a drug molecule comprising an ankyrin repeat domain comprising any of SEQ ID NOs: 15 to 16.

In one embodiment, the recombinant binding protein comprises (i) a binding moiety comprising an ankyrin repeat domain having an amino acid sequence selected from the group consisting of (1) SEQ ID NO: 9 and (2) sequences that have at least about 85% amino acid sequence identity with SEQ ID NO: 9; and (ii) a drug molecule comprising an ankyrin repeat domain having an amino acid sequence selected from the group consisting of (a) SEQ ID NOs: 13 to 17 and (2) sequences that have at least about 85% amino acid sequence identity with SEQ ID NOs: 13 to 17.

In one embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 9, and (2) a drug molecule comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with any of SEQ ID NOs: 13 to 17.

In one embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 9, and (2) a drug molecule comprising an ankyrin repeat domain having at least about 90% amino acid sequence identity with any of SEQ ID NOs: 13 to 17. In another embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 9, and (2) a drug molecule comprising any of SEQ ID NOs: 13 to 17.

In another embodiment, the recombinant binding protein comprises (1) a binding moiety comprising an ankyrin repeat domain having the amino acid sequence of SEQ ID NO: 9, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 9 have been substituted by other amino acids, and (2) a drug molecule comprising an ankyrin repeat domain having an amino acid sequence selected from the group consisting of (a) SEQ ID NOs: 13 to 17 and (2) sequences that have at least about 85% amino acid sequence identity with SEQ ID NOs: 13 to 17.

In another embodiment, the recombinant binding protein comprises (1) a binding moiety comprising an ankyrin repeat domain having the amino acid sequence of SEQ ID NO: 9, wherein optionally up to 15, up to 14, up to 13, up to 12, up to 11, up to 10, up to 9, up to B, up to 7, up to 6, up to 5, up to 4, up to 3, up to 2, or up to 1 amino acids in SEQ 1D NO: 9 have been substituted by other amino adds, and (2) a drug molecule comprising an ankyrin repeat domain having at least about 90% amino acid sequence identity with any of SEQ ID NOs: 13 to 17. In another embodiment, the recombinant binding protein comprises (1) a binding moiety comprising an ankyrin repeat domain having the amino acid sequence of SEQ ID NO: 9, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 9 have been substituted by other amino acids, and (2) a drug molecule comprising any of SEQ ID NOs: 13 to 17.

In another embodiment, the recombinant binding protein comprises (1) a binding moiety comprising an ankyrin repeat domain having SEQ ID NO: 9, and (2) a drug molecule comprising an ankyrin repeat domain comprising any of SEQ ID NOs: 13 to 17. In another embodiment, the recombinant binding protein comprises (1) a binding moiety comprising an ankyrin repeat domain having SEQ ID NO: 9, and (2) a drug molecule comprising an ankyrin repeat domain comprising any of SEQ ID NOs: 15 to 16.

In one embodiment, the recombinant binding protein comprises (i) a binding moiety comprising an ankyrin repeat domain having an amino acid sequence selected from the group consisting of (1) SEQ ID NO: 10 and (2) sequences that have at least about 85% amino acid sequence identity with SEQ ID NO: 10; and (ii) a drug molecule comprising an ankyrin repeat domain having an amino acid sequence selected from the group consisting of (a) SEQ ID NOs: 13 to 17 and (2) sequences that have at least about 85% amino acid sequence identity with SEQ ID NOs: 13 to 17.

In one embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 10, and (2) a drug molecule comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with any of SEQ ID NOs: 13 to 17.

In one embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 10, and (2) a drug molecule comprising an ankyrin repeat domain having at least about 90% amino acid sequence identity with any of SEQ ID NOs: 13 to 17. In another embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 10, and (2) a drug molecule comprising any of SEQ ID NOs: 13 to 17.

In another embodiment, the recombinant binding protein comprises (1) a binding moiety comprising an ankyrin repeat domain having the amino acid sequence of SEQ ID NO: 10, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 10 have been substituted by other amino acids, and (2) a drug molecule comprising an ankyrin repeat domain having an amino acid sequence selected from the group consisting of (a) SEQ ID NOs: 13 to 17 and (2) sequences that have at least about 85% amino acid sequence identity with SEQ ID NOs: 13 to 17.

In another embodiment, the recombinant binding protein comprises (1) a binding moiety comprising an ankyrin repeat domain having the amino acid sequence of SEQ ID NO: 10, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 10 have been substituted by other amino acids, and (2) a drug molecule comprising an ankyrin repeat domain having at least about 90% amino acid sequence identity with any of SEQ ID NOs: 13 to 17. In another embodiment, the recombinant binding protein comprises (1) a binding moiety comprising an ankyrin repeat domain having the amino acid sequence of SEQ ID NO: 10, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 10 have been substituted by other amino acids, and (2) a drug molecule comprising any of SEQ ID NOs: 13 to 17.

In another embodiment, the recombinant binding protein comprises (1) a binding moiety comprising an ankyrin repeat domain having SEQ ID NO: 10, and (2) a drug molecule comprising an ankyrin repeat domain comprising any of SEQ ID NOs: 13 to 17. In another embodiment, the recombinant binding protein comprises (1) a binding moiety comprising an ankyrin repeat domain having SEQ ID NO: 10, and (2) a drug molecule comprising an ankyrin repeat domain comprising any of SEQ ID NOs: 15 to 16.

In one embodiment, the recombinant binding protein comprises (i) a binding moiety comprising an ankyrin repeat domain having an amino acid sequence selected from the group consisting of (1) SEQ ID NO: 11 and (2) sequences that have at least about 85% amino acid sequence identity with SEQ ID NO: 11; and (ii) a drug molecule comprising an ankyrin repeat domain having an amino acid sequence selected from the group consisting of (a) SEQ ID NOs: 13 to 17 and (2) sequences that have at least about 85% amino acid sequence identity with SEQ ID NOs: 13 to 17.

In one embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 11, and (2) a drug molecule comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with any of SEQ ID NOs: 13 to 17.

In one embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 11, and (2) a drug molecule comprising an ankyrin repeat domain having at least about 90% amino acid sequence identity with any of SEQ ID NOs: 13 to 17. In another embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 11, and (2) a drug molecule comprising any of SEQ ID NOs: 13 to 17.

In another embodiment, the recombinant binding protein comprises (1) a binding moiety comprising an ankyrin repeat domain having the amino acid sequence of SEQ ID NO: 11, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 11 have been substituted by other amino acids, and (2) a drug molecule comprising an ankyrin repeat domain having an amino acid sequence selected from the group consisting of (a) SEQ ID NOs: 13 to 17 and (2) sequences that have at least about 85% amino acid sequence identity with SEQ ID NOs: 13 to 17.

In another embodiment, the recombinant binding protein comprises (1) a binding moiety comprising an ankyrin repeat domain having the amino acid sequence of SEQ ID NO: 11, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 11 have been substituted by other amino acids, and (2) a drug molecule comprising an ankyrin repeat domain having at least about 90% amino acid sequence identity with any of SEQ ID NOs: 13 to 17. In another embodiment, the recombinant binding protein comprises (1) a binding moiety comprising an ankyrin repeat domain having the amino acid sequence of SEQ ID NO: 11, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 11 have been substituted by other amino acids, and (2) a drug molecule comprising any of SEQ ID NOs: 13 to 17.

In another embodiment, the recombinant binding protein comprises (1) a binding moiety comprising an ankyrin repeat domain having SEQ ID NO: 11, and (2) a drug molecule comprising an ankyrin repeat domain comprising any of SEQ ID NOs: 13 to 17. In another embodiment, the recombinant binding protein comprises (1) a binding moiety comprising an ankyrin repeat domain having SEQ ID NO: 11, and (2) a drug molecule comprising an ankyrin repeat domain comprising any of SEQ ID NOs: 15 to 16.

In one embodiment, the recombinant binding protein comprises (i) a binding moiety comprising an ankyrin repeat domain having an amino acid sequence selected from the group consisting of (1) SEQ ID NO: 12 and (2) sequences that have at least about 85% amino acid sequence identity with SEQ ID NO: 12; and (i) a drug molecule comprising an ankyrin repeat domain having an amino acid sequence selected from the group consisting of (a) SEQ ID NOs: 13 to 17 and (2) sequences that have at least about 85% amino acid sequence identity with SEQ ID NOs: 13 to 17.

In one embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 12, and (2) a drug molecule comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with any of SEQ ID NOs: 13 to 17.

In one embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 12, and (2) a drug molecule comprising an ankyrin repeat domain having at least about 90% amino acid sequence identity with any of SEQ ID NOs: 13 to 17. In another embodiment, the recombinant binding protein comprises (1) a binding moiety comprising an ankyrin repeat domain having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with SEQ ID NO: 12, and (2) a drug molecule comprising any of SEQ ID NOs: 13 to 17.

In another embodiment, the recombinant binding protein comprises (1) a binding moiety comprising an ankyrin repeat domain having the amino acid sequence of SEQ ID NO: 12, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 12 have been substituted by other amino acids, and (2) a drug molecule comprising an ankyrin repeat domain having an amino acid sequence selected from the group consisting of (a) SEQ ID NOs: 13 to 17 and (2) sequences that have at least about 85% amino acid sequence identity with SEQ ID NOs: 13 to 17.

In another embodiment, the recombinant binding protein comprises (1) a binding moiety comprising an ankyrin repeat domain having the amino acid sequence of SEQ ID NO: 12, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 12 have been substituted by other amino acids, and (2) a drug molecule comprising an ankyrin repeat domain having at least about 90% amino acid sequence identity with any of SEQ ID NOs: 13 to 17. In another embodiment, the recombinant binding protein comprises (1) a binding moiety comprising an ankyrin repeat domain having the amino acid sequence of SEQ ID NO: 12, wherein optionally up to 15, up to 14, up to 13, up to 12, up to 11, up to 10, up to 9, up to 8, up to 7, up to 6, up to 5, up to 4, up to 3, up to 2, or up to 1 amino acids in SEQ ID NO: 12 have been substituted by other amino acids, and (2) a drug molecule comprising any of SEQ ID NOs: 13 to 17.

In another embodiment, the recombinant binding protein comprises (1) a binding moiety comprising an ankyrin repeat domain having SEQ ID NO: 12, and (2) a drug molecule comprising an ankyrin repeat domain comprising any of SEQ ID NOs: 13 to 17. In another embodiment, the recombinant binding protein comprises (1) a binding moiety comprising an ankyrin repeat domain having SEQ ID NO: 12, and (2) a drug molecule comprising an ankyrin repeat domain comprising any of SEQ ID NOs: 15 to 16.

In one embodiment, said drug molecule comprised in any of said recombinant binding proteins described above is a T cell engager drug molecule.

Prodrug Molecules

The prodrug molecules of the present invention comprise the recombinant binding proteins described herein. Thus, the prodrug molecules of the present invention comprise a binding moiety and a drug molecule linked by a protease-cleavable linker. In one embodiment, the drug molecule is a T-cell engager (TCE) molecule comprising a CD3-specific binding domain and a tumor-associated antigen (TAA)-specific binding domain. The prodrug molecules of the present invention may additionally comprise other moieties, such as a half-life extending moiety.

There is no particular restriction on the nature of TAA-specific binding domains that may be used in the prodrug molecules of the present invention. TAA-specific binding domains that may be used in the prodrug molecules of the present invention include any binding domains with binding specificity for a TAA. One example is an EGFR-specific binding domain, such as the binding domain of SEQ ID NO: 27.

The prodrug molecules for use in the present invention may contain a half-life extending moiety. A half-life extending moiety extends the serum halt-life in vivo of a drug molecule, compared to the same molecule without the half-life extending moiety. Examples of half-life extending moieties include, but are not limited to, polyhistidine, Glu-Glu, glutathione S transferase (GST), thioredoxin, protein A, protein G, an immunoglobulin domain, maltose binding protein (MBP), human serum albumin (HSA) binding domain, or polyethylene glycol (PEG). In some instances, the half-life extending moiety may comprise an ankyrin repeat domain with binding specificity for HSA. In other instances, the half-life extending moiety may comprise an immunoglobulin domain, such as an Fc domain, e.g., the Fc domain of human IgG₁, or a variant or derivative thereof. In one embodiment, the half-life extending moiety comprises an ankyrin repeat domain with binding specificity for HSA, wherein said ankyrin repeat domain comprises an amino acid sequence that has at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% amino acid sequence identity with any one of SEQ ID NOs: 65 to 67.

The components of the prodrug molecule may be combined in any order, provided that the binding moiety and the drug molecule are linked by a protease-cleavable linker. In one embodiment, the orientation of the different components in the prodrug molecule is from N-terminus to C-terminus: (TAA binding domain)-(CD3 binding domain)-(protease-cleavable linker)-(binding moiety)-(half-life extending moiety).

Nucleic Acids & Methods

The present invention further relates to a nucleic acid encoding a binding moiety comprising a designed ankyrin repeat domain as defined herein. Examples of such nucleic acids are provided by SEQ ID NOs: 21 to 24. The present invention further relates to a host cell comprising said nucleic acid.

The present invention further relates to a method of making the binding moiety as defined herein, comprising culturing the host cell defined herein under conditions wherein said recombinant binding protein is expressed. In some embodiments, said host cell is a eukaryotic host cell. In other embodiments, said host cell is a prokaryotic host cell. In one embodiment, the method of making the binding moiety comprises culturing the host cell under conditions wherein said recombinant binding protein is expressed, wherein said binding moiety comprises a designed ankyrin repeat domain and wherein said host cell is prokaryotic host cell, such as, for example, E. coli. In another embodiment, the method of making the binding moiety comprises culturing the host cell under conditions wherein said recombinant binding protein is expressed, wherein said binding moiety comprises an antibody and wherein said host cell is a eukaryotic host cell, such as, for example, a CHO cell.

Pharmaceutical Compositions

The invention further relates to pharmaceutical compositions comprising the binding moiety, the recombinant binding protein or the prodrug described herein and a pharmaceutically acceptable carrier or excipient. The pharmaceutical compositions of the present invention may also comprise the nucleic acids described herein and a pharmaceutically acceptable carrier or excipient.

Uses and methods of treatment using said pharmaceutical compositions are also described herein. The methods and uses encompassed by the present invention are described in more detail below. It is noted that the pharmaceutical compositions, methods and uses treat the disease indications that are treated by the drug molecules used to make the pharmaceutical composition.

The pharmaceutical compositions described herein may be prepared using methods known in the art.

The pharmaceutical compositions comprise a pharmaceutically acceptable carrier or excipient. Standard pharmaceutical carriers include a phosphate buffered saline solution, water, emulsions such as an oil/water or water/oil emulsion, and various types of wetting agents.

The pharmaceutical compositions can comprise any other pharmaceutically acceptable ingredients, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, costing agents, colouring agents, desiccants, detergents, diluents, disinfectants, disintegrants, dispersing agents, dissolution enhancing agents, dyes, emollients, emulsifying agents, emulsion stabilizers, fillers, film forming agents, flavour enhancers, flavouring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, UK, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety.

The pharmaceutical compositions can be formulated to achieve a physiologically compatible pH. In some embodiments, the pH of the pharmaceutical composition can be, for example, between about 4 or about 5 and about 8.0, or between about 4.5 and about 7.5, or between about 5.0 and about 7.5. In exemplary embodiments, the pH of the pharmaceutical composition is between about 5.5 and about 7.5.

In one embodiment, the present invention relates to a method of immune cell activation, such as T cell activation or NK cell activation, in a subject in need thereof, the method comprising the step of administering to said subject the pharmaceutical composition as described herein.

In another embodiment, the present invention relates to a method of controlling release of an active drug molecule in vivo comprising administering the pharmaceutical composition as described herein to a subject in need thereof.

In another embodiment, the present invention relates to a method of treating a subject, the method comprising the step of administering an effective amount of a pharmaceutical composition as defined herein, to a subject in need thereof. In some embodiments, the method is a method of treating a proliferative disease. In some embodiments, the method is a method of treating cancer.

In another embodiment, the present invention relates to a pharmaceutical composition as defined herein for use in therapy. Preferably, the pharmaceutical composition as defined herein is provided for use in treating a proliferative disease. In more preferred embodiments, the proliferative disease is cancer.

The pharmaceutical compositions of the invention are typically administered to subjects that have been identified as having a significant risk for adverse effects typically associated with the drug molecule. In some embodiments, the subject is a mammal. In preferred embodiments, the subject is a human.

In some embodiments, a single administration of the pharmaceutical composition may be sufficient. In other embodiments, repeated administration may be necessary. Various factors will impact on the number and frequency of administrations, such as the age and general health of the subject, as well as the nature and typical dosage regime of the drug molecule.

The pharmaceutical compositions described herein can be administered to the subject via any suitable route of administration, such as parenteral, nasal, oral, pulmonary, topical, vaginal, or rectal administration. Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. For additional details, see Pharmaceutics and Pharmacy Practice, J. B. Lippincott Company. Philadelphia, PA, Banker and Chalmers, eds., pages 238-250 (1982), and ASHP Handbook on injectable Drugs, Toissel, 4th ed., pages 622-630 (1986)).

### EXAMPLES

Materials and Methods

Starting materials and reagents disclosed below are known to those skilled in the art, are commercially available and/or can be prepared using well-known techniques.

Materials

Chemicals were purchased from Sigma-Aldrich (USA). Oligonucleotides were from Microsynth (Switzerland). Unless stated otherwise, DNA polymerases, restriction enzymes and buffers were from New England Biolabs (USA) or Fermentas/Thermo Fisher Scientific (USA). Inducible E. coli expression strains were used for cloning and protein production, e.g., E. coli XL1-blue (Stratagene, USA) or BL21 (Novagen, USA). NLC chips for SPR measurements were from BioRad (BioRad, USA). HTRF reagents were from Cisbio (Cisbio, France). Pan-T cell isolation kit was from Miltenyi Biotec (Germany). Cytotoxicity detection (by LDH release) kit was from Roche. Recombinant proteases were from R&D Systems (Minneapolis, USA) or Sigma-Aldrich (USA).

Molecular Biology

Unless stated otherwise, methods are performed according to known protocols (see, e.g., Sambrook J., Fritsch E. F. and Maniatis T., Molecular Cloning: A Laboratory Manual, Cold Spring Harbor Laboratory 1989, New York).

Designed Ankyrin Repeat Protein Libraries

Methods to generate designed ankyrin repeat protein libraries have been described, e.g. in U.S. Pat. No. 7,417,130; Binz et al. 2003, loc. cit.; Binz et al. 2004, loc. cit., By such methods designed ankyrin repeat protein libraries having randomized ankyrin repeat modules and/or randomized capping modules can be constructed. For example, such libraries could accordingly be assembled based on a fixed N-terminal capping module or a randomized N-terminal capping module, one or more randomized repeat modules, and a fixed C-terminal capping module or a randomized C-terminal capping module. Preferably, such libraries are assembled to not have any of the amino acids C. G, M, N (in front of a G residue) and Pat randomized positions of repeat or capping modules.

Furthermore, such randomized modules in such libraries may comprise additional polypeptide loop insertions with randomized amino acid positions. Examples of such polypeptide loop insertions are complement determining region (CDR) loop libraries of antibodies or de novo generated peptide libraries. For example, such a loop insertion could be designed using the structure of the N-terminal ankyrin repeat domain of human ribonuclease L (Tanaka, N., Nakanishi, M, Kusakabe, Y, Goto, Y., Kitade, Y, Nakamura, K. T., EMBO J. 23(30), 3929-3938, 2004) as guidance. In analogy to this ankyrin repeat domain where ten amino acids are inserted in the beta-turn present close to the border of two ankyrin repeats, ankyrin repeat protein libraries may contain randomized loops (with fixed and randomized positions) of variable length (e.g. 1 to 20 amino acids) inserted in one or more beta-turns of an ankyrin repeat domain.

An N-terminal capping module of an ankyrin repeat protein library preferably possesses the RILLAA, RILLKA or RELLKA motif and any such C-terminal capping module of an ankyrin repeat protein library preferably possesses the KLN, KLA or KAA motif.

The design of such an ankyrin repeat protein library may be guided by known structures of an ankyrin repeat domain interacting with a target. Examples of such structures, identified by their Protein Data Bank (PDB) unique accession or identification codes (PDB-IDs), are 1WDY, 3V31, 3V30, 3V2X, 3V2O, 3UXG, 3TWQ-3TWX, 1N11, 1S70 and 2ZGD.

Examples of designed ankyrin repeat protein libraries, such as N2C and N3C designed ankyrin repeat protein libraries, have been described (U.S. Pat. No. 7,417,130; Binz et al. 2003, loc. cit.; Binz et al. 2004, loc. cit.). The digit in N2C and N3C describes the number of randomized repeat modules present between the N-terminal and C-terminal capping modules.

The nomenclature used to define the positions inside the repeat units and modules is based on Binz et al. 2004, loc. cit. with the modification that borders of the ankyrin repeat modules and ankyrin repeat units are shifted by one amino acid position. For example, position 1 of an ankyrin repeat module of Binz et al. 2004 (loc. cit.) corresponds to position 2 of an ankyrin repeat module of the current disclosure and consequently position 33 of an ankyrin repeat module of Binz et al. 2004, loc. cit. corresponds to position 1 of a following ankyrin repeat module of the current disclosure.

EGFR-Specific Binding Domain

EGFR was chosen as exemplary TAA because a human-mouse cross-reactive ankyrin repeat binding domain was available that allowed simultaneous testing of safety and efficacy (i.e. the therapeutic window) in the same models in vivo.

The EGFR-specific binder was selected from DARPin® libraries by Ribosome Display against the biotinylated, full-length extracellular domain (ECD) of human EGFR (L25-S645) in a way similar to the one described by Binz et al. 2004 (loc. cit.).

From round 1 to round 4 target concentrations were decreased. The resulting pools of DARPin molecules were PCR amplified and ligated into a vector for bacterial expression.

Escherichia coli XL1-Blue were transformed with the resulting plasmid pool for isolation of plasmid DNA as described previously (Ref) and for inoculation of expression cultures. Expression cultures were harvested, lysed and the resulting DARPin crude extracts were screened for their ability to bind to the ECD of human EGFR and mouse EGFR-Fc (L25-S647, RnD Systems) by ELISA. Automated washing in-between the individual incubation steps was performed using PBS containing 0.1% Tween-20 (Sigma).

Binders against a non-masked epitope within subdomain III of EGFR were identified by Homogeneous Time Resolved Fluorescence (HTRF). Biotinylated hEGFR (final 4 nM) was pre-Incubated with 10-fold excess of Erbitux® (Merck) for 1 h at room temperature. Detection reagents MAb Anti 6HIS-Tb cryptate and Streptavidin-d2 (CisBio) were diluted according to manufacturer's recommendation and added together with DARPin molecules at 10 nM final. Binding signals were measured at 660 nm and normalized versus 620 nm signals with Infinite M1000 Pro instrument (Tecan).

Binding of EGFR-Specific Ankyrin Repeat Domain to Recombinant Human and Mouse EGFR by Surface Plasmon Resonance (SPR)

All SPR measurements were performed using a ProteOn™ XPR36 Protein Interaction Array System (Bio-Rad). Biotinylated, monovalent anti-EGFR DARPin® was captured on a NLC neutravidin sensor-chip to ˜48 RUs and ˜55 RUs for affinity measurements to hEGFR, or to ˜66 RUs and ˜127 RUs for affinity measurements to mEGFR (ACRObiosystems), respectively. PBS pH7.4 containing 0.005% Tween-20 (Sigma) was used as flow buffer, which was additionally supplemented with 1 mg/ml BSA in the case of mEGFR target. A three-fold dilution series from 0.74 nM to 60 nM of hEGFR or mEGFR was injected for 180 s at a flow rate of 100 ul/min, and dissociation was recorded for 1800 s. The captured DARPin molecules were regenerated by a single pulse of 10 mM glycine-HCl, pH 2.5+1M NaCl. The data was double-referenced using the interspots (surface reference) and a blank injection (buffer reference). Each individual dilution-set was fitted to the Langmuir-1:1-model.

The affinity of the human-mouse cross-reactive EGFR binder to human and mouse EGFR ECD were measured by SPR. Table 2 shows the binding kinetic data for the EGFR-binding DARPin® in monovalent format, indicating a K_{D} of ˜470 pM to human EGFR ECD and a K_{D} of ˜490 pM to mouse EGFR ECD.

TABLE 2
Binding of biotinylated, monovalent DARPin molecules
to recombinant human and mouse EGFR ECD by SPR

| | | ka | kd | KD | Rmax | Chi² |
|---|---|---|---|---|---|---|
| DARPin | target | (1/Ms) | (1/s) | (nM) | (RU) | (RU) |
| anti-EGFR | hEGFR | 4.2 × 10⁵ | 2.0 × 10⁻⁴ | 0.47 | 67.0 | 15.0 |
| | mEGFR | 6.4 × 10⁵ | 3.2 × 10⁻⁴ | 0.49 | 92.3 | 11.0 |

Cleavage Experiments with Recombinant Proteases

Matrix metalloproteinases (R&D Systems) were activated with p-aminophenylmercuric acetate (APMA) as described by the manufacturer before use. Substrate (CD3-PDD CL) was diluted in TBS-CB (50 mM Tris, pH 7.4, 150 mM NaCl, 10 mM CaCl₂), 0.05% Brij-35) to a 2-fold stock concentration of 5 μM. Addition of an equivolume of protease (10 ng/mL-1000 ng/ml concentration, depending on the reaction speed) was used to start the reaction, performed at 37° C. in an appropriately tempered heat-block. In regular intervals a sample of the reaction was withdrawn and immediately mixed with LabChip buffer that contains SDS to denatured all proteins of the mixture and thus stop the reaction. These samples were analyzed with a LabChip HT Protein Express capillary electrophoresis system (Perkin Elmer, USA) for cleavage of the 3-domain CD3-PDD into a 1-domain Binder and a 2-domain active TCE.

Binding to Human EGFR on HCT 116 Tumor Cells and to Human CD3 on Jurkat Wt Cells by FC

The determination of CD3 binding was performed with human CD3 on Jurkat cells, and binding to hEGFR on cells with HCT116 tumor cells, using Flow Cytometer Attune Nxt. A titration of prodrug DARPin molecules was incubated with 200′000 cells per well (for both Jurkat and HCT116 cells) for 30 min at 4° C. After washing, CD3 binding of DARPin® molecule was detected by 1:1000-diluted anti-DARPin® Antibody-Mix (1 h incubation at 4° C.) with the corresponding secondary antibody anti-rabbit IgG Alexa Fluor 488 (30 min incubation at 4° C.), which was added after washing off excess anti-DARPin Antibodies. Cells were subsequently washed and stained for livedead (aqua, 1:1000, thermofisher) and resuspended in Cytofix fixation buffer (BD Biosciences). Median of mean fluorescence intensities of Alexa Fluor 488 binding on live cells were measured by Flow Cytometry and data was plotted using GraphPad Prism 8.

T-Cell Activation

Specificity and potency of CD3-engaging DARPin molecules was assessed in an in vitro short-term T cell activation assay by FACS measuring CD25 activation marker on CD8+ T cells. Therefore, 100′000 human pan-T effector cells and 20′000 HCT116 target cells per well were co-incubated (E:T ratio 5:1) with serial dilutions of prodrug samples in duplicates in presence of 600 μM human serum albumin for 48 hours at 37° C. After 48 h, cells were washed and stained with 1:5000 Live/Dead FITC (Thermo Fisher), 1:250 mouse anti-human CD8 Pacific Blue (BD), and 1:250 mouse anti-human-CD25 PerCP-Cy5.5 (BC96, eBiosciences) antibodies for 30 min at 4C. After washing and fixation, cells were analyzed on a Flow Cytometer Attune Nxt. T cell activation was assessed by measuring CD25-positive cells on Live/Dead-negative and CD8-positive gated T cells. FACS data was analyzed using FlowJo software and data was plotted using GraphPad Prism 8. Assays were performed three times with pan T-cells from three individual human donors.

Tumor Cell Killing: In Vitro Short-Term Cytotoxicity Assay by LDH Release.

Specificity and potency of CD3-engaging DARPin molecules were assessed by an in-vitro short-term cytotoxicity assay by LDH release. Effector and target cells were co-incubated in duplicates in 96-well plates with an E:T ratio of 5:1 in presence of 600 μM human serum albumin (to mimic physiological concentration). Untouched T cells were isolated from human PBMCs by using a pan-T cell isolation Kit (Miltenyi). 100′000 purified pan-T cells (effector cells) and 20′000 HCT116 cells (target cells) per well were incubated with serial dilutions of selected prodrug DARPin molecules. Various controls were included (i.e., T cells only, tumor cells only, Triton control, binding moieties only). After 48 h incubation, cells were spin down and 100 μl per well supernatant and 100 μl per well LDH reaction mixture (LDH detection kit; Roche Applied Science) were incubated for 30 minutes. Absorbance was measured at 492 nm-620 nm by TECAN infinite M1000Pro reader. After background correction, OD values were plotted using GraphPad Prism 8. Assays were performed three times with pan T-cells from three individual human donors.

In Vivo Experiments

In vivo experiments were performed by Transcure SA (TCS) (Archamps, France) using female NOD/Shi-scid/IL-2Rγnull immunodeficient mouse strain (NCG). Mice were humanized using hematopoetic stem cells (CD34+, HLA-B35+) isolated from human cord blood following TCS's proprietary humanization protocol (hu-mice). Humanized mice were selected and enhanced for T-cell, NK cell and myeloid cell population by receiving a hydrodynamic boost based on the transient expression of human cytokines IL-3, IL-4, IL-15, Flt3-L and GM-CSF one week before tumor cell engraftment. Only mice with a humanization rate (hCD45/total CD45) above 25% were used.

HCT-116 tumor cells were expanded in vitro following ATCC recommendations. Following a viability check, tumor cells in logarithmic growth phase were injected in PBS subcutaneously into the right flank in the animals (3×10⁶ cells). Tumor engraftment was defined as experimental day 0 (DO).

All in vivo procedures have been reviewed and approved by the local ethic committee (CELEAG).

### Example 1: Selection of Ankyrin Repeat Domains with Binding Specificity for CD3-Specific Binding Domains

Summary

Using ribosome display (Hanes, J. and Plückthun, A., PNAS 94, 4937-42, 1997), multiple ankyrin repeat domains with binding specificity for the CD3-specific binding domain of bispecific T-cell engager molecules (TCEs) were selected from DARPin® libraries in a way similar to the one described by Binz et al. 2004 (loc. cit.), with specific conditions and additional de-selection steps. The binding and specificity of the selected clones towards the CD3-specific binding domains were assessed by E. coli crude extract Homogeneous Time Resolved Fluorescence (HTRF), indicating that multiple binding proteins were successfully selected that specifically bind to the CD3-specific binding domains. These initially identified binding proteins were further developed to obtain binding proteins with even higher affinity to and/or even lower off-rate from the CD3-specific binding domains of TCEs. For example, the ankyrin repeat domains of SEQ ID NOs: 1 to 12 constitute amino acid sequences of binding proteins comprising an ankyrin repeat domain with binding specificity and high binding affinity to and/or low off-rate from the CD3-specific binding domain of bispecific T-cell engager molecules.

CD3-Specific Binding Domains as Target and Selection Material

CD3-specific binding domains of bispecific TCEs were used as target and selection material. Such target domains were selected from the polypeptides of SEQ ID NOs: 13-17. Target proteins were biotinylated using standard methods.

Selection of Ankyrin Repeat Proteins with Specificity for CD3-Specific Binding Domains by Ribosome Display

Designed ankyrin repeat protein libraries (N2C and N3C) were used in ribosome display selections against the CD3-specific binding domain (SEQ ID NO:13) used as a target (see Binz et al., Nat Biotechnol 22, 575-582 (2004); Zahnd et al., Nat Methods 4, 269-279 (2007); Hanes et al., Proc Natl Acad Sci USA 95, 14130-14135 (1998)). Four selection rounds were performed per target and library. The four rounds of selection employed standard ribosome display selection, using decreasing target concentrations and increasing washing stringency to increase selection pressure from round 1 to round 4 (Binz et al. 2004, loc. cit.). The number of reverse transcription (RT)-PCR cycles after each selection round was continuously reduced, adjusting to the yield due to enrichment of binders. The 3 resulting pools were then subjected to a binder screening.

Selected Clones Bind Specifically to the CD3-Specific Binding Domain of a TCE as Shown by Crude Extract HTRF

Individually selected ankyrin repeat proteins specifically binding to the CD3-specific binding domain of a TCE in solution were identified by a Homogeneous Time Resolved Fluorescence (HTRF) assay using crude extracts of ankyrin repeat protein-expressing Escherichia coli cells using standard protocols. Ankyrin repeat protein clones selected by ribosome display were cloned into a derivative of the pQE30 (Qiagen) expression vector in the format H-C-X, where H denotes a human serum albumin (HSA)-binding domain, C denotes a CD3-binding domain (SEQ ID NO: 13), and X denotes the selected ankyrin repeat proteins. Constructs were transformed into E. coli XL1-Blue (Stratagene), plated on LB-agar (containing 1% glucose and 50 μg/ml ampicillin) and then incubated overnight at 37° C. Single colonies were picked into a 96 well plate (each done in a single well) containing 200 μl growth medium (LB containing 1% glucose and 50 μg/ml ampicillin) and incubated overnight at 37° C., shaking at 800 rpm. 150 μl of TB medium containing 50 μg/ml ampicillin was inoculated with 10 μl of the overnight culture in a fresh 96-deep-well plate. After incubation for 120 minutes at 37° C. and 850 rpm, expression was induced with IPTG (0.5 mM final concentration) and continued for 6 hours. Cells were harvested by centrifugation of the plates, supernatant was discarded and the pellets were frozen at −20° C. overnight before resuspension in 10 μl B-PERII (Thermo Scientific) and incubation for one hour at room temperature with shaking (600 rpm). Then, 160 μl PBS was added and cell debris was removed by centrifugation (3220 g for 10 min). The extract of each lysed done was applied as a 1:800 dilution (final concentration) in PBSTB (PBS supplemented with 0.1% Tween 200 and 0.1% (w/v) BSA, pH 7.4) together with 12.5 nM (final concentration) biotinylated CD3 binding domain, 1:300 (final concentration) of anti-FLAG-D2 HTRF antibody—FRET acceptor conjugate (Cisbio) and 1:300 (final concentration) of anti-strep-Tb antibody FRET donor conjugate (Cisbio, France) to a well of a 384-well plate and incubated for 120 minutes at RT in the dark. The HTRF was read-out on a Tecan M1000 using a 340 nm excitation wavelength and a 620±10 nm emission filter for background fluorescence detection and a 665±10 nm emission filter to detect the fluorescence signal for specific binding. The same lysate was mixed with 12.5 nM (final concentration) biotinylated HSA, 1:300 (final concentration) of anti-FLAG-D2 HTRF antibody—FRET acceptor conjugate (Cisbio) and 1:300 (final concentration) of anti-strep-Tb antibody FRET donor conjugate (Cisbio, France) to a well of a 384-well plate and incubated for 120 minutes at RT in the dark. The HTRF was read-out on a Tecan M1000 using a 340 nm excitation wavelength and a 620±10 nm emission filter for background fluorescence detection and a 665±10 nm emission filter to detect the fluorescence signal for specific binding. The extract of each lysed clone was tested for inhibition of binding to the biotinylated CD3 binding target domain, and unimpeded binding to the biotinylated HSA, in order to assess specific binding to the CD3 binding domain.

Further Analysis and Selection of Binding Proteins with Lower Affinity for Target Protein

A total of 744 binding proteins were initially identified. Based on binding profiles, 176 candidates were selected to be expressed in 96-well format and purified to homogeneity in parallel to DNA sequencing. Candidates were characterized biophysically by size exclusion chromatography. From these, 24 binders were selected and cloned and produced in X-format. Monovalent, purified binders were characterized biophysically by size exclusion chromatography, Sypro-Orange thermal stability assessment (see Niesen et al., Nat Protoc 2, 2212-2221, (2007)). ProteOn surface plasmon resonance (SPR) target affinity assessment, ELISA, target protein-competition HTRF experiments, and/or SDS-PAGE. From these characterized 24 binders. Binder #1 (SEQ ID NO: 1) was selected for affinity-down-tuning.

A panel of down-tuned binders that bind the same epitope of the CD3-binding domain but with different affinities were generated by replacing selected amino acid residues of Binder #1 with alanine. Affinities of down-tuned binding proteins were validated by surface plasmon resonance (SPR). Binder #2 to Binder #12 (SEQ ID NOs: 2 to 12) were generated by this method, derived from the parental Binder #1. Taken together, the following 12 binding proteins (SEQ ID NOs: 1 to 12) derived from this approach constitute binding moieties of the invention;

Binder #1 (SEQ ID NO:1); Binder #2 (SEQ ID NO:2); Binder #3 (SEQ ID NO:3); Binder #4 (SEQ ID NO:4); Binder #5 (SEQ ID NO:5); Binder #6 (SEQ ID NO:6); Binder #7 (SEQ ID NO:7); Binder #8 (SEQ ID NO:8); Binder #9 (SEQ ID NO:9); Binder #10 (SEQ ID NO:10); Binder #11 (SEQ ID NO:11); and Binder #12 (SEQ ID NO:12).

For analysis of biophysical properties of Binder #1 to Binder #12 and for determination of their binding affinities to target proteins (see Example 2), expression vectors were constructed encoding the binding moieties with a His-tag (SEQ ID NO: 25) fused at the N-terminus for easier purification.

High Level and Soluble Expression of the Binding Proteins Chosen for Analysis

For further analysis, the binders were expressed in E. coli cells and purified using their His-tag according to standard protocols. 50 ml of stationary overnight cultures (TB, 1% glucose, 50 mg/I of ampicillin; 37° C.) were used to inoculate 1000 ml cultures (TB, 50 mg/l ampicillin, 37° C.). At an absorbance of 1.0 to 1.5 at 600 nm, the cultures were induced with 0.5 mM IPTG and incubated at 37° C. for 4-5 h while shaking. The cultures were centrifuged, and the resulting pellets were re-suspended in 25 ml of TBS500 (50 mM Tris-HCl, 500 mM NaCl, pH 8) and lysed (sonication or French press). Following the lysis, the samples were mixed with 50 KU DNase/ml and incubated for 15 minutes prior to a heat-treatment step for 30 minutes at 62.5° C., centrifuged and the supernatant was collected and filtrated. Triton X100 (1% (v/v) final concentration) and imidazole (20 mM final concentration) were added to the homogenate. Proteins were purified over a Ni-nitrilotriacetic (Ni-NTA) acid column followed by a size exclusion chromatography on an ÄKTAxpress™ system according to standard protocols and resins known to the person skilled in the art. Highly soluble ankyrin repeat proteins with binding specificity for TCE CD3 binding domain were purified from E. coli culture (up to 200 mg ankyrin repeat protein per litre of culture) with a purity >95% as estimated from 4-12% SDS-PAGE.

### Example 2: SPR Binding Assays

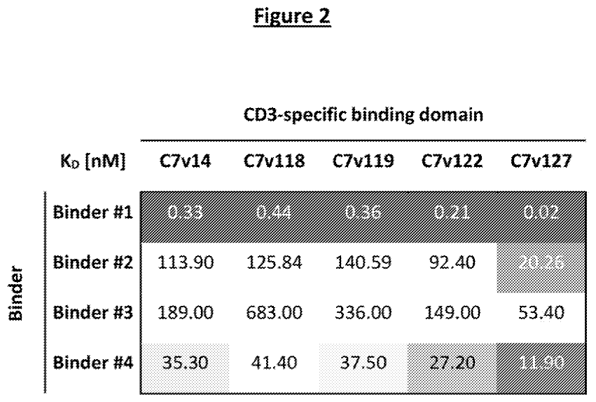

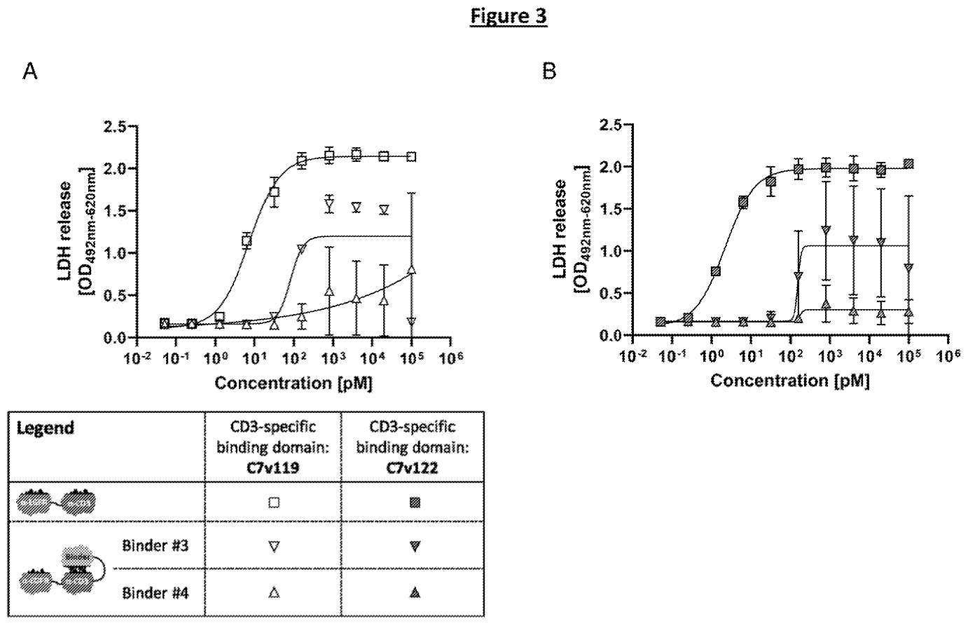

An important feature of a binding moiety of the invention is its affinity towards a drug molecule. Relevant aspects include the off-rate of the binding moiety from the drug molecule and the resulting blocking half-life. Surface plasmon resonance (SPR) assays were used to determine the binding affinity of ankyrin repeat binding domains to the CD3 binding domain of a TCE drug molecule. AN SPR data was generated using a Bio-Rad ProteOn XPR36 instrument with PBS-T (0.005% Tween 20) as running buffer. A new neutravidin sensor chip (NLC) was air-initialized and conditioned according to Bio-Rad manual. SPR data was generated for biotinylated Binders #1 to #4 (as listed in Example 1 above) captured onto the NLC chip and binding to CD3-specific binding domains having SEQ ID NOs: 13 to 17 used as analytes, respectively. The data was generated at 25° C. and with a 1:3 dilution series of target starting at either 50 nM (Binder #1) or 300 nM (Binder #2, #3 and #4), measuring on-rate (kₒₙ), the off-rate (k_{off}) during 40 min and deriving the equilibrium dissociation constant (K_{D}) for Binder #1 and #2. The dissociation constants (K_{D}) for Binder #3 and #4 were obtained by equilibrium fitting. The K_{D} values obtained for all Binder-CD3-specific binding domain combinations are shown in FIG. 2 with more detailed information given in Table 3.

All binders displayed K_{D} values in the double-digit pM to three-digit nM range. Thus, these experiments showed that Binder #1 to #4 cover a large range of affinities to CD3-specific binding domains which are used in TCE drug molecules.

TABLE 3
Binding of different Binders to different
CD3-binding domains by SPR

| | | [symbol] | [symbol] | [symbol] | [symbol] | [symbol] |
|---|---|---|---|---|---|---|
| [symbol] Binder [symbol] | CD3 [symbol] binding [symbol] | [symbol] | [symbol] | [symbol] | [symbol] | [symbol] |
| Binder #1 | SEQ ID NO: [symbol] | [symbol] | [symbol] | [symbol] | [symbol] | [symbol] |
| | SEQ ID NO: [symbol] | [symbol] | [symbol] | [symbol] | [symbol] | [symbol] |
| | SEQ ID NO: [symbol] | [symbol] | [symbol] | [symbol] | [symbol] | [symbol] |
| | SEQ ID NO: [symbol] | [symbol] | [symbol] | [symbol] | [symbol] | [symbol] |
| | SEQ ID NO: [symbol] | [symbol] | [symbol] | [symbol] | [symbol] | [symbol] |
| Binder #2 | SEQ ID NO: [symbol] | [symbol] | [symbol] | [symbol] | [symbol] | [symbol] |
| | SEQ ID NO: [symbol] | [symbol] | [symbol] | [symbol] | [symbol] | [symbol] |
| | SEQ ID NO: [symbol] | [symbol] | [symbol] | [symbol] | [symbol] | [symbol] |
| | SEQ ID NO: [symbol] | [symbol] | [symbol] | [symbol] | [symbol] | [symbol] |
| | SEQ ID NO: [symbol] | [symbol] | [symbol] | [symbol] | [symbol] | [symbol] |
| Binder #3 | SEQ ID NO: [symbol] | [symbol] | [symbol] | [symbol] | [symbol] | [symbol] |
| | SEQ ID NO: [symbol] | [symbol] | [symbol] | [symbol] | [symbol] | [symbol] |
| | SEQ ID NO: [symbol] | [symbol] | [symbol] | [symbol] | [symbol] | [symbol] |
| | SEQ ID NO: [symbol] | [symbol] | [symbol] | [symbol] | [symbol] | [symbol] |
| | SEQ ID NO: [symbol] | [symbol] | [symbol] | [symbol] | [symbol] | [symbol] |
| Binder #4 | SEQ ID NO: [symbol] | [symbol] | [symbol] | [symbol] | [symbol] | [symbol] |
| | SEQ ID NO: [symbol] | [symbol] | [symbol] | [symbol] | [symbol] | [symbol] |
| | SEQ ID NO: [symbol] | [symbol] | [symbol] | [symbol] | [symbol] | [symbol] |
| | SEQ ID NO: [symbol] | [symbol] | [symbol] | [symbol] | [symbol] | [symbol] |
| | SEQ ID NO: [symbol] | [symbol] | [symbol] | [symbol] | [symbol] | [symbol] |

[symbol]

indicates data missing or illegible when filed

### Example 3: CD3-PDD can be Constructed Using Anti-CD3 Binding Domains and Binders with Different Affinities to Each Other

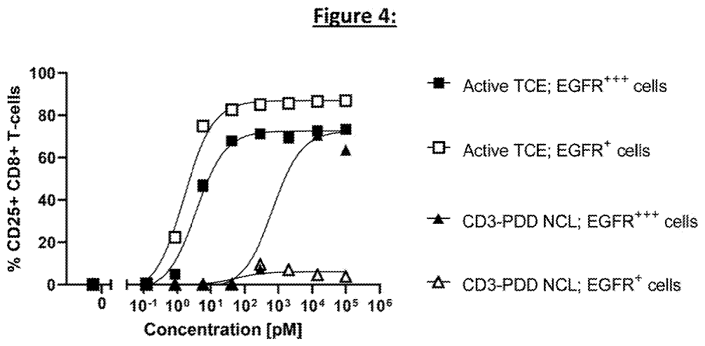

CD3-PDD can be constructed using anti-CD3 binding domains with different affinities to CD3 and different Binders to these. FIG. 3 shows a standard tumor cell killing assay using HCT 116 tumor cells and pan T-cells from one representative out of three donors. Active TCEs (anti-EGFR×anti-CD3), comprising either the anti-CD3 binding domain C7v119 with lower affinity to CD3 or the anti-CD3 binding domain C7v122 with higher affinity to CD3, were compared to their corresponding, CD3-PDD NCL counter-parts containing two different Binders to the anti-CD3 domain (Binder #3 or #4).

In line with the measured affinities of the Binders to the respective CD3-binding domains shown in FIG. 2, Binder #4 with higher affinity towards both CD3-binding domain showed an overall higher masking efficiency than the lower affinity Binder #3. In addition, masking efficiencies of the Binders #3 and #4 in the context of C7v122 CD3-binding domain (FIG. 38) were found to be higher than for constructs with the CD3-binding domain C7v119 (FIG. 3A). EC50 values in pM are given in Table 4.

TABLE 4
EC50 values (pM) obtained for tumor cell killing assays
using HCT 116 tumor cells and pan T-cells. Values are
shown for one representative donor out of three.

| | C7v119 | C7v122 |
|---|---|---|
| | comprising | comprising |
| | constructs | constructs |
| | EC50 (pM) | EC50 (pM) |
| Active TCE | 7.4 | 2.2 |
| CD3-PDD NCL with | 82.1 | >100 |
| Binder #3 | | |
| CD3-PDD NCL with | n.a. | n.a. |
| Binder #4 | | |

n.a.: not applicable

### Example 5: Masking Efficiency of CD3-PDD is Dependent on the Antigen Expression Level on the Target Cells

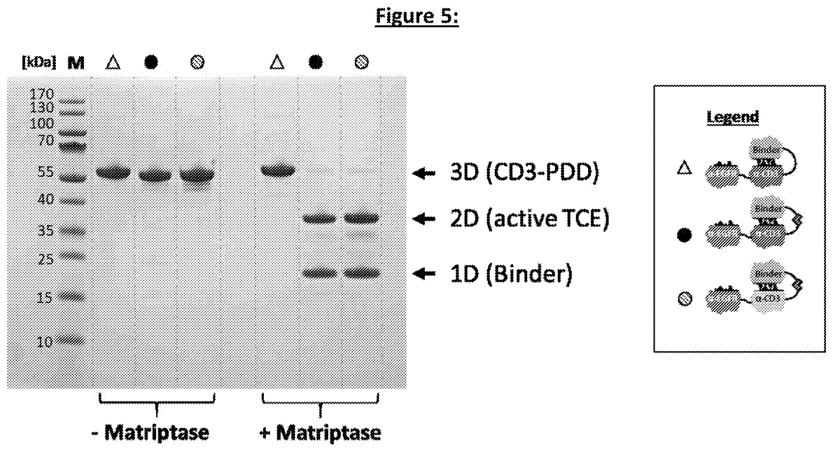

To understand the impact of the tumor-associated antigen (TAA) expression level on the masking efficiency, T-cell activation assays were performed with two different EGFR-expressing tumor cell lines, namely squamous carcinoma cell line A431 and colorectal cancer cell line HCT 116. Quantification by QUIFIKIT resulted in ˜230 k EGFR molecules on A431 cells (EGFR⁺⁺⁺) and ˜18 k EGFR molecules on HCT 116 cells (EGFR⁺). T-cell activation experiments using these two cell lines and either active TCE or a non-cleavable CD3-PDD NCL (both containing the CD3-binding domain binder C7v14) are shown in FIG. 4 and demonstrate that for EGFR mid- to low-expressing HCT 116 cells, the masking window (i.e. the EC50 difference between active TCE and CD3-PDD NCL) is very large, whereas for EGFR high-expressing A431 cells, the masking window is only around 200-fold. This dependence of the masking efficiency on the tumor antigen expression level has also been described by Geiger et al., (Nat Communication, 2020), for a similar prodrug concept as the one described here.

Table 5. summarizes the EC50 values in pM measured in T-cell activation assay.

TABLE 5
EC50 values (pM) in T-cell activation assay using
either EGFR^{+++ A431} or EGFR⁺ HCT
116 tumor cells and pan T-cells. Values are shown
for one representative donor out of two.

| | EGFR⁺⁺⁺ | EGFR⁺ |
|---|---|---|
| | (A431) | (HCT 116) |
| | EC50 (pM) | EC50 (pM) |
| Active TCE | 3.7 | 1.6 |
| CD3-PDD NCL | 679.2 | 53.2 |
| with Binder #3 | | |

### Example 6: Recombinant Proteases Cleave the CD3-PDD CL Efficiently In Vitro

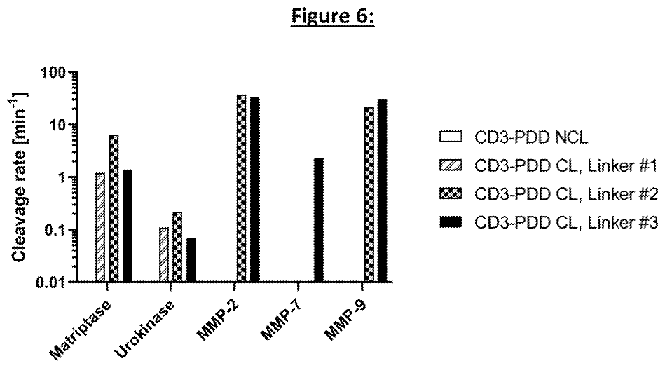

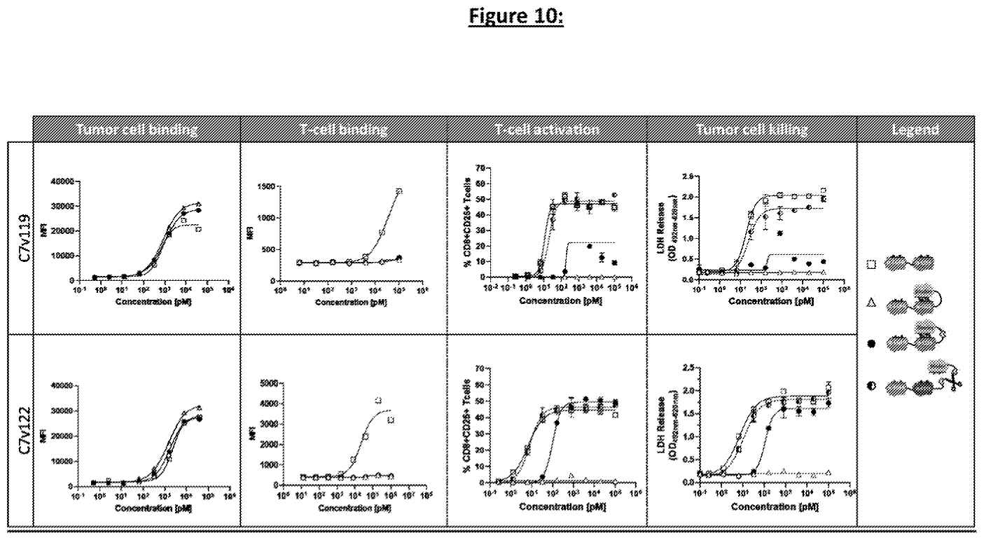

Non-cleavable prodrugs CD3-PDD (NCL) as well as cleavable CD3-PDD (CL) containing the cleavable linker #2 and either the CD3-binding domain C7v119 or C7v122 were incubated with or without Matriptase (1:10′000 enzyme:substrate ratio) and cleavage was analyzed after incubation for 20 h at 37° C. on sodium dodecyl sulfate polyacrylamide gel electrophoresis (SDS-PAGE) (FIG. 5). CD3-PDD NCL was not impacted by the matriptase, whereas the two CD3-PDD CL comprising either C7v119 or C7v122 were cleaved predominantly Into the 1-domain (1D) Binder and the 2-domain (2D) active TCE. The cleaved CD3-PDD CL were then utilized as pre-cleaved controls in T-cell activation and tumor cell killing assays as shown in FIG. 10 and proved to be almost as functional as the active TCE.

In a next step, CD3-PDD CL containing three different cleavable linker sequences (Linker #1-#3), as well as CD3-PDD NCL were investigated in detail with five different tumor-associated proteases, namely matriptase, urokinase, MMP-2, -7, and -9. Cleavage rates of each construct with each protease were determined by recording cleavage progress curves and analyzing the resulting 1D and 2D fragments on a capillary electrophoretic separation device (LabChip). Cleavage rates (cleaved CD3-PDD CL molecules per enzyme molecules and minute) were calculated and are shown in FIG. 6. Linker #1 exhibited cleavage only by matriptase and urokinase, matrix-metalloproteases (MMPs) were not able to cleave these constructs. In contrast, Linker #2 was cleaved efficiently by all proteases investigated except MMP-7, whereas Linker #3 was cleaved by all five proteases. As the overall cleavage rate of Linker #2 was superior to Linker #3, constructs containing Linker #2 were chosen for further investigation.

### Example 7: CD3-PDD are Only Active in Presence of Both TAA on Target Cells and Cleavage of Linker

The concept of conditionally activated CD3-prodrugs constitutes a form of a logic AND gate: only if event 1 (the TAA-binding domain binding to the TAA), as well as event 2 (tumor associated proteases un-block the CD3-binder by cleaving the linker) are present, the drug can form the trimolecular complex between T-cell, drug and target cell. Hence, if either event 1 or 2 is missing, the CD3-PDD should not be able to form this trimolecular complex.

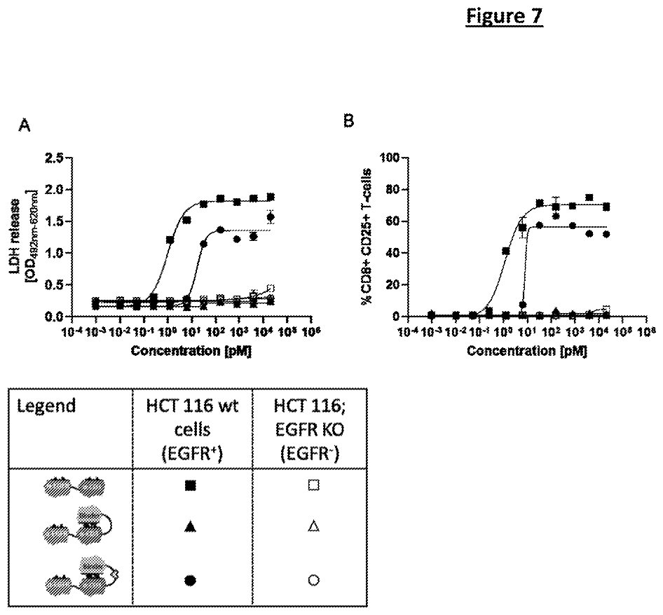

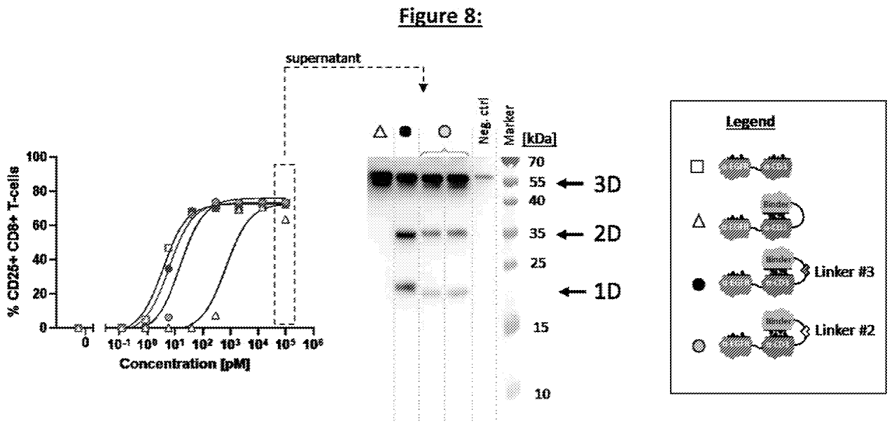

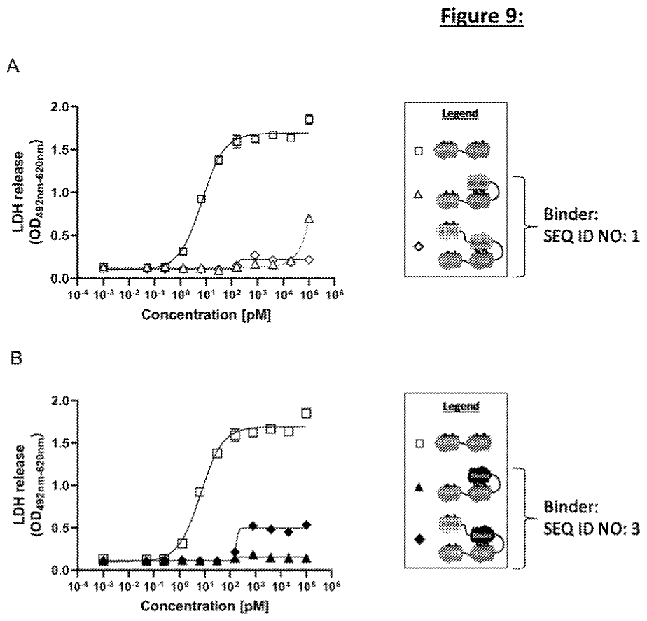

To investigate this hypothesis, we checked whether CD3-PDD NCL and CL showed T-cell activation and tumor cell killing in absence of the TAA on the tumor cell (event 1) or with a masked CD3-binding domain (event 2). Therefore, an EGFR-knockout cell line of HCT 116 was generated and tested alongside with the wildtype cell line for tumor cell killing and T-cell activation (see FIGS. 7A and 7B, respectively). In absence of the TAA EGFR, none of the constructs exhibited tumor cell killing or T-cell activation, confirming that event 1 is a pre-requisite for activity. For CD3-PDD NCL containing a non-cleavable linker between CD3 binding domain and the Binder, no activity was observed in terms of tumor cell killing or T-cell activation. In contrast, for the CD3-PDD CL containing the cleavable Linker #2 tumor cell killing and T-cell activation could be observed, however at lower potency and efficacy levels. This observation is attributed to the partial cleavage of the CD3-PDD CL molecule by proteases that are secreted by the cells in the in vitro assay (see FIG. 8).

Table 6. summarizes the EC50 values (in pM) measured in tumor cell killing and T-cell activation assays using either EGFR⁺ or EGFR KO HCT116 tumor cells and pan T-cells of one representative donor.

TABLE 6
EC50 values (pM) measured in T-cell activation and tumor
cell killing assays using HCT 116 tumor cells and pan
T-cells. Values are shown for one representative donor.

| | HCT 116 N1 cells (EGFR*) | | HCT 116 cells (EGFR KO) | |
|---|---|---|---|---|
| | Tumor cell | T-cell | Tumor cell | T-cell |
| | killing | activation | killing | activation |
| | EC50 (pM) | EC50 (pM) | EC50 (pM) | EC50 (pM) |
| Active TCE | 1.0 | 1.2 | n.a. | n.a. |
| CD3-PDD CL | 16.7 | ca. 7-8 | n.a. | n.a. |
| CD3-PDD | >30 | >100 | n.a. | n.a. |
| NCL | | | | |

n.a.: not applicable

### Example 8: Proteases that are Secreted in In Vitro Cell Assays Lead to Activation of the CD3-PDD CL

It has been observed that CD3-PDD CL reproducibly exhibited T-cell activation and tumor cell killing activity that were higher than the one observed for the non-cleavable CD3-PDD NCL. The prevailing hypothesis was that proteases secreted by the tumor cells or the T-cells were cleaving and hence activating the CD3-PDD CL. To confirm this hypothesis, supernatant of a T-cell activation experiment of pan T-cells and A431 tumor cells was harvested at the end of the experiment (i.e. after 48 h incubation) and analyzed by immunoprecipitation and Western blot (FIG. 8). Indeed, it was found that constructs with two different linker sequences (Linker #2 and #3) were cleaved to a significant degree, as exemplified by the presence of 1-domain (1D) and 2-domain (2D) bands at 35 kDa and 17 kDa, respectively. The degree of cleavage also correlated with the EC50 of the respective CD3-PDD CL construct. In contrast. CD3-PDD NCL with non-cleavable linker did not show any cleavage and displayed a masking window of >100-fold (EC50 difference between active TCE and CD3-PDD NCL).

### Example 9: CD3-PDD Constructs can be Half-Life Extended by Attaching an Anti-HSA Binding Domain to their C-Terminus

CD3-PDD constructs can readily be half-life extended (HLE) by attaching an anti-HSA binding domain to their C-terminus. This brings the advantage that a long-lived CD3-PD molecule can be converted into a short-lived, active TCE (anti-TAA×anti-CD3) upon cleavage of the protease-cleavable linker by tumor-associated proteases. The short-lived, active TCE will be rapidly cleared when leaving the tumor and entering circulation resulting in less side-effects.

In order to investigate a potentially negative steric effect of HSA on the masking efficiency of the Binder on the anti-CD3 domain, we compared active TCE, non-half-life extended CD3-PDD NCL and half-life extended CD3-PDD NCL in a standard tumor cell killing assay using pan T-cells and HCT 116 tumor cells in the presence of 600 μM HSA. Both non-HLE and HLE CD3-PDD constructs were studied in the context of two different Binders in order to potentially counter-act steric hindrance by making the blocking interaction tighter: Binder #1 exhibiting high affinity (<1 nM K_{D}) towards CD3-binding domain and Binder #3 exhibiting lower affinity towards CD3-binding domain (>100 nM K_{D}), see FIGS. 9A and 9B, respectively.

CD3-PDD constructs containing the lower affinity Binder #3 (>100 nM K_{D}) were found to show a minor reduction of masking efficiency with the anti-HSA binding domain attached to their C-terminus, thus indicating that indeed the presence of HSA may have a slightly negative steric effect on the Binder-anti-CD3 domain-interaction. However, this slight loss in masking efficiency can be overcome by choosing a higher affinity Binder for the CD3-PDD constructs.

### Example 10: Cell Binding

Binding to human EGFR on HCT 116 tumor cells and to human CD3 on Jurkat wt cells was assessed by FC, where DARPin molecules were detected with fluorescently labeled anti-DARPin antibody. For this purpose, active TCE (anti-EGFR×anti-CD3) and CD3-PDD constructs with CL or NCL were compared head to head. As expected, binding HCT 116 cells was comparable with EC50 values in the range of 350-600 μM for all tested constructs (see FIG. 10, first column and Table 7).

Binding to Jurkat wt cells was found to be slightly stronger for the active TCE containing the higher affinity CD3-binder C7v122 (SEQ ID NO: 16) (EC50=7.9 nM) compared to the active TCE containing the lower affinity CD3-binder C7v119 (SEQ ID NO: 15) (EC50>10 nM; see FIG. 10, second column). CD3-PDD constructs with CL or NCL did not show binding to T-cells via CD3, thus confirming the functionality of the masking concept.

TABLE 7
EC50 values (pM) for binding of active TCE or CD3-
PDD constructs to HCT 116 tumor cells and EC50 values
(nM) for binding to Jurkat cells. Values are shown
for one representative donor out of three

| | C7v119 | | C7v122 | |
|---|---|---|---|---|
| | comprising constructs | | comprising constructs | |
| | HCT 116 | Jurkat | HCT 116 | Jurkat |
| | EC50 (pM) | EC50 (nM) | EG50 pM) | EC50 (nM) |
| Active TCE | 427.0 | >10 | 518.4 | 7.9 |
| Pre-cleaved | n.d. | n.d. | n.d. | n.d. |
| CD3-PDD CL | | | | |
| CD3-PDD CL | 353.5 | n.a. | 410.5 | n.a. |
| CD3-PDD NCL | 502.9 | n.a. | 593.1 | n.a. |

### Example 11: T-Cell Activation/Tumor Cell Killing Assays

For the T-cell activation and tumor cell killing assays, target tumor cells and effector T-cells (pan T-cells from healthy blood donors) were combined at an effector to target cell ratio of 5:1, prodrug samples were added, and the mixtures were incubated for 48 hours at 37° C. Supernatant was analyzed for LDH release of killed tumor cells and the levels of activation markers (CD25) on CD8+ T-cells were determined by FACS (using CD25 Monoclonal Antibody (BC96), PerCP-Cyanine5.5, eBioscience™). Various controls were included (i.e., T cells only, tumor cells only, Triton control, binding moieties only).

Assays were performed three times with pan T-cells from three individual human donors.

For this purpose, active TCE (anti-EGFR×anti-CD3), CD3-PDD NCL, CD3-PDD CL and pre-cleaved CD3-PDD CL either containing the CD3-binding domain C7v119 (SEQ ID NO: 15) (lower affinity for CD3) or C7v122 (SEQ ID NO: 18) (higher affinity for CD3) were compared head to head. For all CD3-PDD constructs Binder #4 (SEQ ID NO: 4) was used to mask the CD3-binding domain C7v119 or C7v122.

Active TCE and pro-cleaved CD3-PDD CL displayed EC50 values in the single-digit to low double-digit pM range in T-cell activation (see FIG. 10, third column and Table 8) and in tumor cell killing assays (see FIG. 10, fourth column and Table 8). In comparison to the active TCE and pre-cleaved CD3-PDD CL, C03-PDD CL constructs displayed ca. 10-20-fold higher EC50 values in T-cell activation and tumor cell killing assays, presumably due to their limited cleavage by proteases present under the tested in vitro conditions.

In addition, lower potency and lower efficacy were observed for the CD3-PDD CL constructs comprising the CD3-binding domain C7v119 (see FIG. 10, upper row and Table 8) in comparison to constructs comprising the C7v122 domain (see FIG. 10, lower row and Table 8). Thus, C7v122-based constructs were moved into the PoC in vivo study along with respective controls.

CD3-PDD NCL constructs did not lead to T-cell activation nor to tumor cell killing.

TABLE 8
EC50 values (pM) in T-cell activation and tumor cell killing
assays using HCT 116 tumor cells and pan T-cells. Values
are shown for one representative donor out of three

| | C7v119 | | C7v122 | |
|---|---|---|---|---|
| | comprising constructs | | comprising constructs | |
| | T-cell | Tumor cell | T-cell | Tumor cell |
| | activation | killing | activation | killing |
| | EC50 (pM) | EC50 (pM) | EC50 pM) | EC50 (pM) |
| Active TCE | 10.8 | 18.5 | 6.8 | 7.2 |
| Pre-cleaved | 19.1 | 24.3 | 8.1 | 11.1 |
| CD3-PDD CL | | | | |
| CD3-PDD CL | >100 | >100 | 99.1 | 109.6 |
| CD3-PDD NCL | n.a. | n.a. | n.a. | n.a. |

### Example 12: The Cleavable CD3-PDD is Efficacious and Well Tolerated In Vivo in CD34+ Hu-Mice Engrafted with HCT 116 Tumor Cells

Constructs using the higher affinity CD3-binding domain C7v122 (SEQ ID NO: 16) tested in Examples 10 and 11 for T-cell binding, T-cell activation and tumor cell killing (FIG. 10) were selected for a proof-of-concept in vivo study. For this in vivo study, none of the molecules was half-life extended.

The aim of the in vivo study was to assess tolerability and efficacy of the cleavable CD3-PDD in a human colon carcinoma xenograft model (HCT 116) using immunodeficient mice humanized with hematopoietic stem cells (CD34+) and optimized for the presence of human myeloid cells. Due to the mouse cross-reactivity of the EGFR-binder, this animal model allowed to assess for a therapeutic window. i.e. for both anti-tumor efficacy and safety at the same time. Twenty-four hu-mice were engrafted subcutaneously with 3×10⁶ HCT-116 cells at D0. Hydrodynamic plasmid delivery (cytokine boost of IL-3, IL-4, Flt3L, IL-15 and GM-CSF) was performed 7 days before tumor cell engraftment. When tumors reached an average volume of 35 mm³, mice were randomized into 4 groups of 6 mice according to their tumor volume, hCD3 T cell count, humanization rate and cord blood donor as follows:
Group 1: Vehicle control (PBS+0.05% Tween-20)
Group 2: Active TCE control at 30 nmol/kg
Group 3: CD3-PDD with protease-cleavable linker (CL) at 30 nmol/kg
Group 4: CD3-PDD with non-cleavable linker (NCL) at 30 nmol/kg

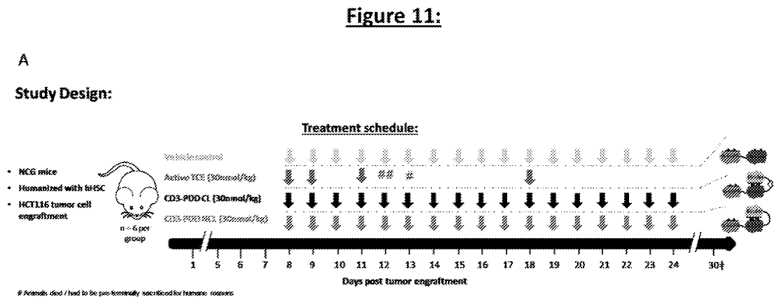

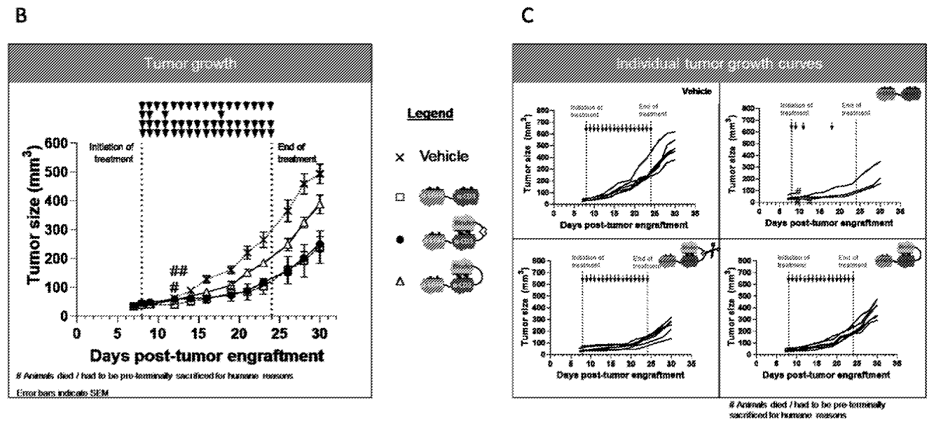

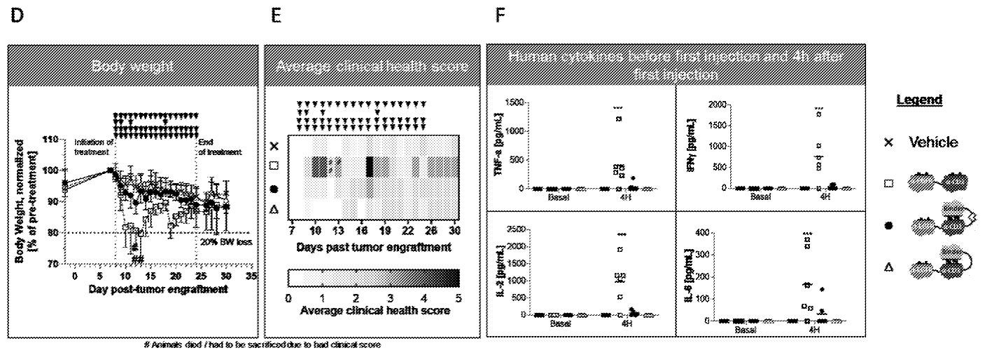

Daily intraperitoneal injections were initiated at D8 for 17 days for all groups, except for the active T-cell engager control group which received only 4 injections at D8, D9, D11 and D18 due to the induction of treatment-related toxicities. Blood was collected before tumor cell engraftment at D-1 and 4 hours after the first treatment (D8) for plasma collection and downstream cytokines measurement. Tumor volume was monitored three times a week. Body weight and clinical health scores were measured three times per week until treatment initiation and daily during the treatment period. The cleavable CD3-PDD demonstrated a robust anti-tumor activity, similar to the one observed with active, non-blocked TCE (FIG. 11B). Anti-tumor efficacy of CD3-NCL was in-between active TCE and vehicle. Most importantly, however, both CD3-PDD CL and NCL could be dosed daily without signs of toxicity, whereas dosing of active TCE had to be stopped due to strong toxicity, leading even to the loss of 3/6 animals. This toxicity is represented in strong loss of body weight (BW) of the group treated with active TCE already after two injections (FIG. 11D) as well as in a quick deterioration of the clinical health score of animals in this group (FIG. 11E). Cytokines that were determined from serum of mice before injection and 4 h after injection of the first dose showed almost no elevation of cytokines for the mice in groups treated with CD3-PDD CL and NCL, but significantly elevated levels for the animals treated with active TCE. In summary, animals treated with CD3-PDD CL showed robust anti-tumor efficacy without the adverse toxicity effects of active TCE.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications, patents, and GenBank sequences cited in this disclosure are incorporated by reference in their entirety. To the extent the material incorporated by reference contradicts or is Inconsistent with this specification, the specification will supersede any such material. The citation of any references herein is not an admission that such references are prior art to the present invention.

Those skilled in the art will recognize or be able to ascertain using no more than routine experimentation, many equivalents to the specific embodiments of the invention described herein. Such equivalents are intended to be encompassed by the following claims.

Sequences

| SEQ | Name of the | | | |
|---|---|---|---|---|
| ID | His-tagged | | | |
| NO | version | Description | Comments | Sequence |
| 1 | Binder #1 | Designed ankyrin | 06E05 | DLGKKLLQAARAGQLDEVRELLKAGADVNAKDAKGLTPLHLAAYHGHLEIVEVL |
| | | repeat domain | | LKAGADVNAKDVYGWTPLHIAAASGHLEIVEVLLKAGADVNAKDWLGWTPLHL |
| | | | | AASHGHLEIVEVLLKAGADVNAQDKSGKTPADLAARAGHQDIAEVLQKAA |
| 2 | Binder #2 | Designed ankyrin | 06E05v03 | DLGKKLLQAARAGQLDEVRELLKAGADVNAKDAKGLTPLHLAAYHGHLEIVEVL |
| | | repeat domain | | LKAGADVNAKDVAGWTPLHIAAASGHLEIVEVLLKAGADVNAKDWLGWTPLHL |
| | | | | AASHGHLEIVEVLLKAGADVNAQDKSGKTPADLAARAGHQDIAEVLQKAA |
| 3 | Binder #3 | Designed ankyrin | 06E05v05 | DLGKKLLQAARAGQLDEVRELLKAGADVNAKDAKGLTPLHLAAYHGHLEIVEVL |
| | | repeat domain | | LKAGADVNAKDVYGWTPLHIAAASGHLEIVEVLLKAGADVNAKDALGWTPLHL |
| | | | | AASHGHLEIVEVILKAGADVNAQDKSGKTPADLAARAGHQDIAEVLQKAA |
| 4 | Binder #4 | Designed ankyrin | 06E05v07 | DLGKKLLQAARAGQLDEVRELLKAGADVNAKDAKGLTPLHLAAYHGHLEIVEVL |
| | | repeat domain | | LKAGADVNAKDVYGWTPLHIAAASGHLEIVEVLLKAGADVNAKDWLGWTPLHL |
| | | | | AASAGHLEIVEVLLKAGADVNAQDKSGKTPADLAARAGHQDIAEVLQKAA |
| 5 | Binder #5 | Designed ankyrin | 06E05v30 | DLGKKLLQAARAGQLDEVRELLKAGADVNAKDAKGLTPLHLAAYHGHLEIVEVL |
| | | repeat domain | | LKAGADVNAKDVYGWTPLHIAAASGHLEIVEVLLKAGADVNAKDWLGITPLHLA |
| | | | | ASHGHLEIVEVLLKAGADVNAQDKSGKTPADLAARAGHQDIAEVLQKAA |
| 6 | Binder #6 | Designed ankyrin | 06E05v31 | DLGKKLLQAARAGQLDEVRELLKAGADVNAKDAKGLTPLHLAAYHGHLEIVEVL |
| | | repeat domain | | LKAGADVNAKDVAGWTPLHIAAASGHLEIVEVLLKAGADVNAKDWLGITPLHLA |
| | | | | ASHGHLEIVEVLLKAGADVNAQDKSGKTPADLAARAGHQDIAEVLQKAA |
| 7 | Binder #7 | Designed ankyrin | 06E05v32 | DLGKKLLQAARAGQLDEVRELLKAGADVNAKDAKGLTPLHLAAYHGHLEIVEVL |
| | | repeat domain | | LKAGADVNAKDVYGWTPLHIAAASGHLEIVEVLLKAGADVNAKDALGITPLHLA |
| | | | | ASHGHLEIVEVLLKAGADVNAQDKSGKTPADLAARAGHQDIAEVLQKAA |
| 8 | Binder #8 | Designed ankyrin | 06E05v33 | DLGKKLLQAARAGQLDEVRELLKAGADVNAKDAKGLTPLHLAAYHGHLEIVEVL |
| | | repeat domain | | LKAGADVNAKDVYGWTPLHIAAASGHLEIVEVLLKAGADVNAKDWLGITPLHLA |
| | | | | ASAGHLEIVEVILKAGADVNAQDKSGKTPADLAARAGHQDIAEVLQKAA |
| 9 | Binder #9 | Designed ankyrin | 06E05v34 | DLGKKLLQAARAGQLDEVRELLKAGADVNAKDAKGLTPLHLAAYHGHLEIVEVL |
| | | repeat domain | | LKAGADVNAKDVYGWTPLHIAAASGHLEIVEVLLKAGADVNAKDWLGATPLHL |
| | | | | AASHGHLEIVEVLLKAGADVNAQDKSGKTPADLAARAGHQDIAEVLQKAA |
| 10 | Binder #10 | Designed ankyrin | 06E05v35 | DLGKKLLQAARAGQLDEVRELLKAGADVNAKDAKGLTPLHLAAYHGHLEIVEVL |
| | | repeat domain | | LKAGADVNAKDVAGWTPLHIAAASGHLEIVEVLLKAGADVNAKDWLGATPLHL |
| | | | | AASHGHLEIVEVLLKAGADVNAQDKSGKTPADLAARAGHQDIAEVLQKAA |
| 11 | Binder #11 | Designed ankyrin | 06E05v36 | DLGKKLLQAARAGQLDEVRELLKAGADVNAKDAKGLTPLHLAAYHGHLEIVEVL |
| | | repeat domain | | LKAGADVNAKDVYGWTPLHIAAASGHLEIVEVLLKAGADVNAKDALGATPLHLA |
| | | | | ASHGHLEIVEVLLKAGADVNAQDKSGKTPADLAARAGHQDIAEVLQKAA |
| 12 | Binder #12 | Designed ankyrin | 06E05v37 | DLGKKLLQAARAGQLDEVRELLKAGADVNAKDAKGLTPLHLAAYHGHLEIVEVL |
| | | repeat domain | | LKAGADVNAKDVYGWTPLHIAAASGHLEIVEVLLKAGADVNAKDWLGATPLHL |
| | | | | AASAGHLEIVEVLLKAGADVNAQDKSGKTPADLAARAGHQDIAEVLQKAA |
| 13 | | CD3-specific | hC22_88B02v14 | DLGQKLLEAAWAGQDDEVRILLAAGADVNAKNSRGWTPLHTAAQTGHLEIFEV |
| | | binding domain | C7v14 | LLKAGADVNAKNDKRVTPLHLAAALGHLEIVEVLLKAGADVNARDSWGTTPAD |
| | | | | LAAKYGHGDIAEVLQKAA |
| 14 | | CD3-specific | hC22_88802v118 | DLGQKLLEAAWAGQDDEVRELLKAGADVNAKDSQGWTPLHTAAQTGHLEIFE |
| | | binding domain | C7v118 | VLLKAGADVNAKDDKGVTPLHLAAALGHLEIVEVLLKAGADVNAQDSWGTTPA |
| | | | | DLAAKYGHEDIAEVLQKAA |
| 15 | | CD3-specific | hC22_88802v119 | DLGQKLLEAAWAGQDDEVRELLKAGADVNAKNSRGWTPLHTAAQTGHLEIFE |
| | | binding domain | C7v119 | VLLKAGADVNAKDDKGVTPLHLAAALGHLEIVEVLLKAGADVNAQDSWGTTPA |
| | | | | DLAAKYGHEDIAEVLQKAA |
| 16 | | CD3-specific | hC22_88802v122 | DLGQKLLEAAWAGQDDEVRELLKAGADVNAKNSRGWTPLHTAAQTGHLEIFE |
| | | binding domain | C7v122 | VLLKAGADVNAKNDKRVTPLHLAAALGHLEIVEVLLKAGADVNARDSWGTTPA |
| | | | | DLAAKYGHQDIAEVLQKAA |
| 17 | | CD3-specific | hC22_88802v127 | DLGQKLLEAAWAGQLDEVRILLKAGADVNAKNSRGWTPLHTAAQTGHLEIFEV |
| | | binding domain | C7v127 | LLKAGADVNAKTNKRVTPLHLAAALGHLEIVEVLLKAGADVNARDTWGTTPADL |
| | | | | AAKYGHRDIAEVLQKAA |
| 18 | Linker #1 | Cleavable linker | | GSGSGGSGGLSGRSDNHGGSGGSGGS |
| | | sequence #1 | | |
| 19 | Linker #2 | Cleavable linker | | GSGGGGPQASTGRSGGGGGGS |
| | | sequence #2 | | |
| 20 | Linker #3 | Cleavable linker | | GSGSPQGIWGPLSGRSDNHGSGS |
| | | sequence #3 | | |
| 21 | | Nucleic acid | 06E05 | GACTTAGGAAAGAAATTGCTGCAAGCCGCACGGGCCGGTCAACTTGATGA |
| | | encoding a | | GGTGCGCGAATTATTGAAGGCAGGTGCAGACGTGAACGCTAAAGACGCTA |
| | | designed ankyrin | | AGGGACTTACTCCTTTACACTTAGCGGCCTATCATGGTCATTTGGAAATTGT |
| | | repeat domain | | GGAGGTCCTGTTGAAGGCTGGTGCCGACGTGAACGCCAAAGATGTTTACG |
| | | | | GTTGGACCCCATTACACATTGCTGCCGCCTCGGGACATCTGGAAATTGTTG |
| | | | | AGGTTCTGCTTAAAGCTGGCGCAGACGTTAATGCCAAGGACTGGTTGGGG |
| | | | | TGGACGCCCTTACACCTGGCCGCGTCACATGGACATTTAGAGATTGTAGAA |
| | | | | GTCCTGTTAAAGGCGGGGGGGGACGTTAATGCCCAAGACAAAAGTGGCAA |
| | | | | AACACCAGCGGATCTGGCCGCTCGTGCTGGACACCAGGACATTGCTGAAG |
| | | | | TGCTGCAGAAGGCAGCG |
| 22 | | Nucleic acid | 06E05v03 | GACTTAGGAAAGAAATTGCTGCAAGCCGCACGCGCCGGTCAACTTGATGA |
| | | encoding & | | GGTGCGCGAATTATTGAAGGCAGGTGCAGACGTGAACGCTAAAGACGCTA |
| | | designed ankyrin | | AGGGACTTACTCCTTTACACTTAGCGGCCTATCATGGTCATTTGGAAATTGT |
| | | repeat domain | | GGAGGTCCTGTTGAAGGCTGGCGCCGACGTGAACGCCAAAGATGTTGCAG |
| | | | | GTTGGACCCCATTACACATTGCTGCCGCCTCGGGACATCTGGAAATTGTTG |
| | | | | AGGTTCTGCTTAAAGCTGGCGCAGACGTTAATGCCAAGGACTGGTTGGGG |
| | | | | TGGACGCCCTTACACCTGGCCGCGTCACATGGACATTTAGAGATTGTAGAA |
| | | | | GTCCTGTTAAAGGCGGGGGGGGACGTTAATGCCCAAGACAAAAGTGGCAA |
| | | | | AACACCAGCGGATCTGGCCGCTCGTGCTGGACACCAGGACATTGCTGAAG |
| | | | | TGCTGGAGAAGGCAGCG |
| 23 | | Nucleic acid | 06E05v05 | GAGTTAGGAAAGAAATTGCTGCAAGCCGCACGCGCGGGTCAACTTGATGA |
| | | encoding a | | GGTGCGCGAATTATTGAAGGCAGGTGCAGACGTGAACGCTAAAGACGCTA |
| | | designed ankyrin | | AGGGACTTACTCCTTTACACTTAGCGGCCTATCATGGTCATTTGGAAATTGT |
| | | repeat domain | | GGAGGTCCTGTTGAAGGCTGGGGCCGACGTGAACGCCAAAGATGTTTACG |
| | | | | GTTGGACCCCATTACACATTGCTGCCGCCTCGGGACATCTGGAAATTGTTG |
| | | | | AGGTTCTGCTTAAAGCTGGCGCAGACGTTAATGCCAAGGACGCATTGGGG |
| | | | | TGGACGCCCTTACACCTGGCCGCGTCACATGGACATTTAGAGATTGTAGAA |
| | | | | GTCCTGTTAAAGGCGGGGGGGGACGTTAATGCCCAAGACAAAAGTGGCAA |
| | | | | AACACCAGCGGATCTGGCCGCTCGTGCTGGACACCAGGACATTGCTGAAG |
| | | | | TGCTGCAGAAGGCAGCG |
| 24 | | Nucleic acid | 06E05v07 | GACTTAGGAAAGAAATTGCTGCAAGCCGCACGCGCCGGTCAACTTGATGA |
| | | encoding a | | GGTGCGCGAATTATTGAAGGGAGGTGCAGACGTGAACGCTAAAGACGCTA |
| | | designed ankyrin | | AGGGACTTACTCCTTTACACTTAGCGGCCTATCATGGTCATTTGGAAATTGT |
| | | repeat domain | | GGAGGTCCTGTTGAAGGCTGGTGCCGACGTGAACGCCAAAGATGTTTACG |
| | | | | GTTGGACCCCATTACACATTGCTGCCGCCTCGGGACATCTGGAAATTGTTG |
| | | | | AGGTTCTGCTTAAAGCTGGCGCAGACGTTAATGCCAAGGACTGGTTGGGG |
| | | | | TGGACGCCCTTACACCTGGCCGCGTCAGCAGGACATTTAGAGATTGTAGAA |
| | | | | GTCCTGTTAAAGGGGGGCGCGGACGTTAATGCCCAAGACAAAAGTGGCAA |
| | | | | AACACCAGCGGATCTGGCCGCTCGTGCTGGACACCAGGACATTGCTGAAG |
| | | | | TGCTGCAGAAGGCAGCG |
| 25 | | His-tag | | MRGSHHHHHHGS |
| 28 | | His-tag | | GSHHHHHH |
| 27 | | EGFR binding | | MGSDLGYKLLRAAFHGQDDEVRILLAAGADVNAKDLIGQTPLHNAAWVGHLEI |
| | | domain used in | | VEVLLKAGADVNAKDYYGNTPLHLAAHDGHLEIVEVLLKAGADVNAQDTWGET |
| | | the CD3-PDD | | PADLATNAGHEDIAEVLQKAA |
| | | constructs | | |
| 28 | | Non-cleavable | NCL | XXPTPTPTTPTPTPTTPTPTPTGS wherein XX represents GS or SG |
| | | linker | | |
| 29 | | Full sequence of | EGFR-C7v14- | MGSQLGYKLLRAAFHGQDDEVRILLAAGADVNAKDLIGQTPLHNAAWVGHLEI |
| | | active TCE | 6His | VEVLLKAGADVNAKDYYGNTPLHLAAHDGHLEIVEVLLKAGADVNAQDTWGET |
| | | | | PADLATNAGHEDIAEVLQKAAGSPTPTPTTPTPTPTTPTPTPTGSDLGQKLLEA |
| | | | | AWAGQDDEVRILLAAGADVNAKNSRGWTPLHTAAQTGHLEIFEVLLKAGADVN |
| | | | | AKNDKRVTPLHLAAALGHLEIVEVLLKAGADVNARDSWGTTPADLAAKYGHGD |
| | | | | IAEVLQKAAGSHHHHHH |
| 30 | | Full sequence of | EGFR-C7v119- | MGSDLGYKLLRAAFHGQDDEVRILLAAGADVNAKDLIGQTPLHNAAWVGHLEI |
| | | active TCE | 6His | VEVLLKAGADVNAKDYYGNTPLHLAAHDGHLEIVEVLLKAGADVNAQDTWGET |
| | | | | PADLATNAGHEDIAEVLQKAAGSPTPTPTTPTPTPTTPTPTPTGSDLGQKLLEA |
| | | | | AWAGQDDEVRELLKAGADVNAKNSRGWTPLHTAAQTGHLEIFEVLLKAGADV |
| | | | | NAKDDKGVTPLHLAAALGHLEIVEVLLKAGADVNAQDSWGTTPADLAAKYGHE |
| | | | | DIAEVLQKAAGSHHHHHH |
| 31 | | Full sequence of | EGFR-C7v122- | MGSDLGYKLLRAAFHGQDDEVRILLAAGADVNAKDLIGQTPLHNAAWVGHLEI |
| | | active TCE | 6His | VEVLLKAGADVNAKDYYGNTPLHLAAHDGHLEIVEVLLKAGADVNAQDTWGET |
| | | | | PADLATNAGHEDIAEVLQKAAGSPTPTPTTPTPTPTTPTPTPTGSDQLGQKLLEA |
| | | | | AWAGQDDEVRELLKAGADVNAKNSRGWTPLHTAAQTGHLEIFEVLLKAGADV |
| | | | | NAKNDKRVTPLHLAAALGHLEIVEVLLKAGADVNARDSWGTTPADLAAKYGHQ |
| | | | | DIAEVLQKAAGSHHHHHH |
| 32 | | Full sequence of | EGFR-C7v14- | MGSDLGYKLLRAAFHGQDDEVRILLAAGADVNAKDLIGQTPLHNAAWVGHLEI |
| | | CD3-PDD NCL | [NCL2]-06E05- | VEVLLKAGADVNAKDYYGNTPLHLAAHDGHLEIVEVLLKAGADVNAQDTWGET |
| | | | 6His | PADLATNAGHEDIAEVLQKAAGSPTPTPTTPTPTPTTPTPTPTGSDLGQKLLEA |
| | | | | AWAGQDDEVRILLAAGADVNAKNSRGWTPLHTAAQTGHLEIFEVLLKAGADVN |
| | | | | AKNDKRVTPLHLAAALGHLEIVEVLLKAGADVNARDSWGTTPADLAAKYGHGD |
| | | | | IAEVLQKAASGPTPTPTTPTPTPTTPTPTPTGSDLGKKLLQAARAGQLDEVREL |
| | | | | LKAGADVNAKDAKGLTPLHLAAYHGHLEIVEVLLKAGADVNAKDVYGWTPLHIA |
| | | | | AASGHLEIVEVLLKAGADVNAKDWLGWTPLHLAASHGHLEIVEVLLKAGADVN |
| | | | | AQDKSGKTPADLAARAGHQDIAEVLQKAAGSHHHHHH |
| 33 | | Full sequence of | EGFR-C7v14- | MGSDLGYKLLRAAFHGQDDEVRILLAAGADVNAKDLIGQTPLHNAAWVGHLEI |
| | | CD3-PDD NCL | [NOL2]- | VEVLLKAGADVNAKDYYGNTPLHLAAHDGHLEIVEVLLKAGADVNAQDTWGET |
| | | | 06E05v05-6His | PADLATNAGHEDIAEVLQKAAGSPTPTPTTPTPTPTTPTPTPTGSDLGQKLLEA |
| | | | | AWAGQDDEVRILLAAGADVNAKNSRGWTPLHTAAQTGHLEIFEVLLKAGADVN |
| | | | | AKNDKRVTPLHLAAALGHLEIVEVLLKAGADVNARDSWGTTPADLAAKYGHGD |
| | | | | IAEVLQKAASGPTPTPTTPTPTPTTPTPTPTGSDLGKKLLQAARAGQLDEVREL |
| | | | | LKAGADVNAKDAKGLTPLHLAAYHGHLEIVEVLLKAGADVNAKDVYGWTPLHIA |
| | | | | AASGHLEIVEVLLKAGADVNAKDALGWTPLHLAASHGHLEIVEVLLKAGADVNA |
| | | | | QDKSGKTPADLAARAGHQDIAEVLQKAAGSHHHHHH |
| 34 | | Full sequence of | EGFR-C7v14- | MGSDLGYKLLRAAFHGQDDEVRILLAAGADVNAKQLIGQTPLHNAAWVGHLEI |
| | | half-life extended | (NCL1]-06E05- | VEVLLKAGADVNAKDYYGNTPLHLAAHDGHLEIVEVLLKAGADVNAQDTWGET |
| | | CD3-PDD NCL | HSA-6His | PADLATNAGHEDIAEVLQKAAGSPTPTPTTPTPTPTTPTPTPTGSDLGQKLLEA |
| | | | | AWAGQDDEVRILLAAGADVNAKNSRGWTPLHTAAQTGHLEIFEVLLKAGADVN |
| | | | | AKNDKRVTPLHLAAALGHLEIVEVLLKAGADVNARDSWGTTPADLAAKYGHGD |
| | | | | IAEVLQKAASGPTPTPTTPTPTPTTPTPTPTGSDLGKKLLQAARAGQLDEVREL |
| | | | | LKAGADVNAKDAKGLTPLHLAAYHGHLEIVEVLLKAGADVNAKDVYGWTPLHIA |
| | | | | AASGHLEIVEVLLKAGADVNAKDWLGWTPLHLAASHGHLEIVEVLLKAGADVN |
| | | | | AQDKSGKTPADLAARAGHQDIAEVLQKAAGSPTPTPTTPTPTPTTPTPTPTGS |
| | | | | DLGKKLLEAARAGQDDEVRELLKAGADVNAKDYFSHTPLHLAARNGHLKIVEV |
| | | | | LLKAGADVNAKDFAGKTPLHLAAADGHLEIVEVLLKAGADVNAQDIFGKTPADIA |
| | | | | ADAGHEDIAEVLQKAAGSHHHHHH |
| 35 | | Full sequence of | EGFR-C7v14- | MGSDLGYKLLRAAFHGQDDEVRILLAAGADVNAKDLIGQTPLHNAAWVGHLEI |
| | | half-life extended | [NCL1]- | VEVLLKAGADVNAKDYYGNTPLHLAAHDGHLEIVEVLLKAGADVNAQDTWGET |
| | | CD3-PDD NCL | 06E05v05-HSA- | PADLATNAGHEDIAEVLQKAAGSPTPTPTTPTPTPTTPTPTPTGSDLGQKLLEA |
| | | | 6His | AWAGQDDEVRILLAAGADVNAKNSRGWTPLHTAAQTGHLEIFEVLLKAGADVN |
| | | | | AKNDKRVTPLHLAAALGHLEIVEVLLKAGADVNARDSWGTTPADLAAKYGHGD |
| | | | | IAEVLQKAASGPTPTPTTPTPTPTTPTPTPTGSDLGKKLLQAARAGQLDEVREL |
| | | | | 37LKAGADVNAKDAKGLTPLHLAAYHGHLEIVEVLLKAGADVNAKDVYGWTPL |
| | | | | HIAAASGHLEIVEVLLKAGADVNAKDALGWTPLHLAASHGHLEIVEVLLKAGAD |
| | | | | VNAQDKSGKTPADLAARAGHQDIAEVLQKAAGSPTPTPTTPTPTPTTPTPTPT |
| | | | | GSDLGKKLLEAARAGQDDEVRELLKAGADVNAKDYFSHTPLHLAARNGHLKIV |
| | | | | EVLLKAGADVNAKDFAGKTPLHLAAADGHLEIVEVLLKAGADVNAQDIFGKTPA |
| | | | | DIAADAGHEDIAEVLQKAAGSHHHHHH |
| 36 | | Full sequence of | EGFR-C7v119- | MGSDLGYKLLRAAFHGQDDEVRILLAAGADVNAKDLIGQTPLHNAAWVGHLEI |
| | | CD3-PDD NCL | [NCL1]- | VEVLLKAGADVNAKDYYGNTPLHLAAHDGHLEIVEVLLKAGADVNAQDTWGET |
| | | | 06E05v05-6His | PADLATNAGHEDIAEVLQKAAGSPTPTPTTPTPTPTTPTPTPTGSDLGQKLLEA |
| | | | | AWAGQDDEVRELLKAGADVNAKNSRGWTPLHTAAQTGHLEIFEVLLKAGADV |
| | | | | NAKDDKGVTPLHLAAALGHLEIVEVLLKAGADVNAQDSWGTTPADLAAKYGHE |
| | | | | DIAEVLQKAASGPTPTPTTPTPTPTTPTPTPTGSDLGKKLLQAARAGQLDEVRE |
| | | | | LLKAGADVNAKDAKGLTPLHLAAYHGHLEIVEVLLKAGADVNAKDVYGWTPLHI |
| | | | | AAASGHLEIVEVLLKAGADVNAKDALGWTPLHLAASHGHLEIVEVLLKAGADVN |
| | | | | AQDKSGKTPADLAARAGHQDIAEVLQKAAGSHHHHHH |
| 37 | | Full sequence of | EGFR-C7v122- | MGSDLGYKLLRAAFHGQDDEVRILLAAGADVNAKDLIGQTPLHNAAWVGHLEI |
| | | CD3-PDD NCL | [NCL1]- | VEVLLKAGADVNAKDYYGNTPLHLAAHDGHLEIVEVLLKAGADVNAQDTWGET |
| | | | 06E05v05-6HIs | PADLATNAGHEDIAEVLQKAAGSPTPTPTTPTPTPTTPTPTPTGSDLGQKLLEA |
| | | | | AWAGQDDEVRELLKAGADVNAKNSRGWTPLHTAAQTGHLEIFEVLLKAGADV |
| | | | | NAKNDKRVTPLHLAAALGHLEIVEVLLKAGADVNARDSWGTTPADLAAKYGHQ |
| | | | | DIAEVLQKAASGPTPTPTTPTPTPTTPTPTPTGSDLGKKLLQAARAGQLDEVRE |
| | | | | LLKAGADVNAKDAKGLTPLHLAAYHGHLEIVEVLLKAGADVNAKQVYGWTPLHI |
| | | | | AAASGHLEIVEVLLKAGADVNAKDALGWTPLHLAASHGHLEIVEVLLKAGADVN |
| | | | | AQDKSGKTPADLAARAGHQDIAEVLQKAAGSHHHHHH |
| 38 | | Full sequence of | EGFR-C7v119- | MGSDLGYKLLRAAFHGQDDEVRILLAAGADVNAKDLIGQTPLHNAAWVGHLEI |
| | | CD3-PDD NCL | [NCL1]- | VEVLLKAGADVNAKDYYGNTPLHLAAHDGHLEIVEVLLKAGADVNAQDTWGET |
| | | | 06E05vQ7-6His | PADLATNAGHEDIAEVLQKAAGSPTPTPTTPTPTPTTPTPTPTGSDLGQKLLEA |
| | | | | AWAGQDDEVRELLKAGADVNAKNSRGWTPLHTAAQTGHLEIFEVLLKAGADV |
| | | | | NAKDDKGVTPLHLAAALGHLEIVEVLLKAGADVNAQDSWGTTPADLAAKYGHE |
| | | | | DIAEVLQKAASGPTPTPTTPTPTPTTPTPTPTGSDLGKKLLQAARAGQLDEVRE |
| | | | | LLKAGADVNAKDAKGLTPLHLAAYHGHLEIVEVLLKAGADVNAKDVYGWTPLHI |
| | | | | AAASGHLEIVEVLLKAGADVNAKDWLGWTPLHLAASAGHLEIVEVLLKAGADVN |
| | | | | AQDKSGKTPADLAARAGHQDIAEVLQKAAGSHHHHHH |
| 39 | | Full sequence of | EGFR-C7v122- | MGSDLGYKLLRAAFHGQDDEVRILLAAGADVNAKDLIGQTPLHNAAWVGHLEI |
| | | CD3-PDD NCL | [NCL1]- | VEVLLKAGADVNAKDYYGNTPLHLAAHDGHLEIVEVLLKAGADVNAQQTWGET |
| | | | 06E05v07-6His | PADLATNAGHEDIAEVLQKAAGSPTPTPTTPTPTPTTPTPTPTGSDLGQKLLEA |
| | | | | AWAGQDDEVRELLKAGADVNAKNSRGWTPLHTAAQTGHLEIFEVLLKAGADV |
| | | | | NAKNDKRVTPLHLAAALGHLEIVEVLLKAGADVNARDSWGTTPADLAAKYGHQ |
| | | | | DIAEVLQKAASGPTPTPTTPTPTPTTPTPTPTGSDLGKKLLQAARAGQLDEVRE |
| | | | | LLKAGADVNAKDAKGLTPLHLAAYHGHLEIVEVLLKAGADVNAKDVYGWTPLHI |
| | | | | AAASGHLEIVEVLLKAGADVNAKDWLGWTPLHLAASAGHLEIVEVLLKAGADVN |
| | | | | AQDKSGKTPADLAARAGHQDIAEVLQKAAGSHHHHHH |
| 40 | | Full sequence of | EGFR-C7v14- | MGSDLGYKLLRAAFHGQDDEVRILLAAGADVNAKDLIGQTPLHNAAWVGHLEI |
| | | CD3-POD CL with | [Linker#1]-06E05- | VEVLLKAGADVNAKDYYGNTPLHLAAHDGHLEIVEVLLKAGADVNAQDTWGET |
| | | cleavable linker | 6His | PADLATNAGHEDIAEVLQKAAGSPTPTPTTPTPTPTTPTPTPTGSDLGQKLLEA |
| | | #1 | | AWAGQDDEVRILLAAGADVNAKNSRGWTPLHTAAQTGHLEIFEVLLKAGADVN |
| | | | | AKNDKRVTPLHLAAALGHLEIVEVLLKAGADVNARDSWGTTPADLAAKYGHGO |
| | | | | IAEVLQKAAGSGSGGSGGLSGRSDNHGGSGGSGGSDLGKKLLQAARAGQLD |
| | | | | EVRELLKAGADVNAKDAKGLTPLHLAAYHGHLEIVEVLLKAGADVNAKDVYGW |
| | | | | TPLHIAAASGHLEIVEVLLKAGADVNAKDWLGWTPLHLAASHGHLEIVEVLLKA |
| | | | | GADVNAQDKSGKTPADLAARAGHQDIAEVLQKAAGSHHHHHH |
| 41 | | Full sequence of | EGFR-C7v14- | MGSDLGYKLLRAAFHGQDDEVRILLAAGADVNAKDLIGQTPLHNAAWVGHLEI |
| | | CD3-PDD CL with | [Linker#2]- | VEVLLKAGADVNAKDYYGNTPLHLAAHDGHLEIVEVLLKAGADVNAQDTWGET |
| | | cleavable linker | 06E05v05-6HIs | PADLATNAGHEDIAEVLOKAAGSPTPTPTTPTPTPTTPTPTPTGSDLGQKLLEA |
| | | #2 | | AWAGQDDEVRILLAAGADVNAKNSRGWTPLHTAAQTGHLEIFEVLLKAGADVN |
| | | | | AKNDKRVTPLHLAAALGHLEIVEVLLKAGADVNARDSWGTTPADLAAKYGHGD |
| | | | | IAEVLQKAAGSGGGGPQASTGRSGGGGGGSDLGKKLLQAARAGQLDEVRELL |
| | | | | KAGADVNAKDAKGLTPLHLAAYHGHLEIVEVLLKAGADVNAKOVYGWTPLHIAA |
| | | | | ASGHLEIVEVLLKAGADVNAKDALGWTPLHLAASHGHLEIVEVLLKAGADVNAQ |
| | | | | DKSGKTPADLAARAGHQDIAEVLOKAAGSHHHHHH |
| 42 | | Full sequence of | EGFR-C7v14- | MGSDLGYKLLRAAFHGQDDEVRILLAAGADVNAKDLIGQTPLHNAAWVGHLEI |
| | | CD3-PDD CL with | [Linker#3]- | VEVLLKAGADVNAKDYYGNTPLHLAAHDGHLEIVEVLLKAGADVNAQDTWGET |
| | | cleavable linker | 06E05vQ5-6His | PADLATNAGHEDIAEVLOKAAGSPTPTPTTPTPTPTTPTPTPTGSDLGQKLLEA |
| | | #3 | | AWAGQDDEVRILLAAGADVNAKNSRGWTPLHTAAQTGHLEIFEVLLKAGADVN |
| | | | | AKNDKRVTPLHLAAALGHLEIVEVLLKAGADVNARDSWGTTPADLAAKYGHGD |
| | | | | IAEVLQKAAGSGSPQGIWGPLSGRSDNHGSGSDLGKKLLQAARAGQLDEVRE |
| | | | | LLKAGADVNAKDAKGLTPLHLAAYHGHLEIVEVLLKAGADVNAKOVYGWTPLHI |
| | | | | AAASGHLEIVEVLLKAGADVNAKDALGWTPLHLAASHGHLEIVEVLLKAGADVN |
| | | | | AQDKSGKTPADLAARAGHQDIAEVLQKAAGSHHHHHH |
| 43 | | Full sequence of | EGFR-C7v119- | MGSDLGYKLLRAAFHGQDDEVRILLAAGADVNAKDLIGQTPLHNAAWVGHLEI |
| | | CD3-PDD CL with | [Linker#2]- | VEVLLKAGADVNAKDYYGNTPLHLAAHDGHLEIVEVLLKAGADVNAQOTWGET |
| | | cleavable linker | 06E05v07-6His | PADLATNAGHEDIAEVLQKAAGSPTPTPTTPTPTPTTPTPTPTGSDLGQKLLEA |
| | | #2 | | AWAGQDDEVRELLKAGADVNAKNSRGWTPLHTAAQTGHLEIFEVLLKAGADV |
| | | | | NAKDDKGVTPLHLAAALGHLEIVEVLLKAGADVNAQDSWGTTPADLAAKYGHE |
| | | | | DIAEVLQKAASGGGGGPQASTGRSGGGGGGSDLGKKLLQAARAGQLDEVRE |
| | | | | LLKAGADVNAKDAKGLTPLHLAAYHGHLEIVEVLLKAGADVNAKDVYGWTPLHI |
| | | | | AAASGHLEIVEVLLKAGADVNAKDWLGWTPLHLAASAGHLEIVEVLLKAGADVN |
| | | | | AQDKSGKTPADLAARAGHQDIAEVLOKAAGSHHHHHH |
| 44 | | Full sequence of | EGFR-C7v122- | MGSDLGYKLLRAAFHGQDDEVRILLAAGADVNAKDLIGOTPLHNAAWVGHLEI |
| | | CD3-PDD CL with | [Linker#2]- | VEVLLKAGADVNAKDYYGNTPLHLAAHDGHLEIVEVLLKAGADVNAQDTWGET |
| | | cleavable linker | 06E05vQ7-6His | PADLATNAGHEDIAEVLQKAAGSPTPTPTTPTPTPTTPTPTPTGSDLGQKLLEA |
| | | #2 | | AWAGQDDEVRELLKAGADVNAKNSRGWTPLHTAAQTGHLEIFEVLLKAGADV |
| | | | | NAKNDKRVTPLHLAAALGHLEIVEVLLKAGADVNARDSWGTTPADLAAKYGHQ |
| | | | | DIAEVLQKAASGGGGGPQASTGRSGGGGGGSDLGKKLLQAARAGQLDEVRE |
| | | | | LLKAGADVNAKDAKGLTPLHLAAYHGHLEIVEVLLKAGADVNAKOVYGWTPLHI |
| | | | | AAASGHLEIVEVLLKAGADVNAKDWLGWTPLHLAASAGHLEIVEVLLKAGADVN |
| | | | | AQDKSGKTPADLAARAGHQDIAEVLQKAAGSHHHHHH |
| 45 | | Ankyrin repeat | Binder #1 | KDAKGLTPLHLAAYHGHLEIVEVLLKAGADVNA |
| | | module | | |
| 46 | | Ankyrin repeat | Binder #1 | KDVYGWTPLHIAAASGHLEIVEVLLKAGADVNA |
| | | module | | |
| 47 | | Ankyrin repeat | Binder #1 | KDWLGWTPLHLAASHGHLEIVEVLLKAGADVNA |
| | | module | | |
| 48 | | Ankyrin repeat | Binder #2 | KDAKGLTPLHLAAYHGHLEIVEVLLKAGADVNA |
| | | module | | |
| 49 | | Ankyrin repeat | Binder #2 | KDVAGWTPLHIAAASGHLEIVEVLLKAGADVNA |
| | | module | | |
| 50 | | Ankyrin repeat | Binder #2 | KDWLGWTPLHLAASHGHLEIVEVLLKAGADVNA |
| | | module | | |
| 51 | | Ankyrin repeat | Binder #3 | KDAKGLTPLHLAAYHGHLEIVEVLLKAGADVNA |
| | | module | | |
| 52 | | Ankyrin repeat | Binder #3 | KOVYGWTPLHIAAASGHLEIVEVLLKAGADVNA |
| | | module | | |
| 53 | | Ankyrin repeat | Binder #3 | KDALGWTPLHLAASHGHLEIVEVLLKAGADVNA |
| | | module | | |
| 54 | | Ankyrin repeat | Binder #4 | KDAKGLTPLHLAAYHGHLEIVEVLLKAGADVNA |
| | | module | | |
| 55 | | Ankyrin repeat | Binder #4 | KDVYGWTPLHIAAASGHLEIVEVLLKAGADVNA |
| | | module | | |
| 56 | | Ankyrin repeat | Binder #4 | KDWLGWTPLHLAASAGHLEIVEVLLKAGADVNA |
| | | module | | |
| 57 | | Ankyrin repeat | Binder #5 | KDWLGITPLHLAASHGHLEIVEVLLKAGADVNA |
| | | module | | |
| 58 | | Ankyrin repeat | Binder #6 | KDWLGITPLHLAASHGHLEIVEVLLKAGADVNA |
| | | module | | |
| 59 | | Ankyrin repeat | Binder #7 | KDALGITPLHLAASHGHLEIVEVLLKAGADVNA |
| | | module | | |
| 60 | | Ankyrin repeat | Binder #8 | KDWLGITPLHLAASAGHLEIVEVLLKAGADVNA |
| | | module | | |
| 61 | | Ankyrin repeat | Binder #9 | KDWLGATPLHLAASHGHLEIVEVLLKAGADVNA |
| | | module | | |
| 62 | | Ankyrin repeat | Binder #10 | KDWLGATPLHLAASHGHLEIVEVLLKAGADVNA |
| | | module | | |
| 63 | | Ankyrin repeat | Binder #11 | KDALGATPLHLAASHGHLEIVEVLLKAGADVNA |
| | | module | | |
| 64 | | Ankyrin repeat | Binder #12 | KDWLGATPLHLAASAGHLEIVEVLLKAGADVNA |
| | | module | | |
| 65 | | HSA-specific | | DLGKKLLEAARAGQDDEVRELLKAGADVNAKDYFSHTPLHLAARNGHLKIVEV |
| | | binding domain | | LLKAGADVNAKDFAGKTPLHLAANEGHLEIVEVLLKAGADVNAQDIFGKTPADIA |
| | | | | ADAGHEDIAEVLOKAA |
| 66 | | HSA-specific | | DLGKKLLEAARAGQDDEVRELLKAGADVNAKDYFSHTPLHLAARNGHLKIVEV |
| | | binding domain | | LLKAGADVNAKDFAGKTPLHLAAADGHLEIVEVLLKAGADVNAQDIFGKTPADIA |
| | | | | ADAGHEDIAEVLOKAA |
| 67 | | HSA-specific | | DLGKKLLEAARAGQDDEVRELLKAGADVNAKDYFSHTPLHLAARNGHLKIVEV |
| | | binding domain | | LLKAGADVNAKDFAGKTPLHLAADAGHLEIVEVLLKAGADVNAQDIFGKTPADIA |
| | | | | ADAGHEDIAEVLOKAA |
| 68 | | Ankyrin repeat | | xDxxGxTPLHLAxxxGxxxIVxVLLxxGADVNA |
| | | module | | wherein ″x″ denotes any amino acid (preferably not cysteine, glycine, or |
| | | | | proline) |
| 69 | | Ankyrin repeat | | xDxxGxTPLHLAAxxGHLEIVEVLLKxGADVNA |
| | | module | | wherein ″x″ in position 1. 3, 4, 6, 14, 15 denotes any amino acid |
| | | | | (preferably not cysteine, glycine, or proline), and ″x″ in position 27 is |
| | | | | selected from the group consisting of asparagine, histidine, or tyrosine |
| 70 | | N-cap | | GSDLGKKLLQAARAGQLDEVRELLKAGADVNA |
| 71 | | N-cap | | GSDLGKKLLQAARAGQLDEVRILLKAGADVNA |
| 72 | | N-cap | | GSDLGKKLLQAARAGQLDEVRILLAAGADVNA |
| 73 | | N-Cap | | GSDLGXKLLQAAXXGOLDEVRILLKAGADVNA |
| | | (randomized) | | |
| 74 | | C-cap | | QDKFGKTPADIAADNGHEDIAEVLOKLN |
| 75 | | C-cap | | QDKSGKTPADLAARAGHQDIAEVLOKAA |
| 76 | | C-cap | | QDSSGFTPADLAALVGHEDIAEVLQKAA |
| 77 | | C-cap | | QDXXGXTPADLAARXGHQDIAEVLQKAA |
| | | (randomized) | | |
| 78 | | PT-rich peptide | | GSPTPTPTTPTPTPTTPTPTPTTPTPTPTTPTPTPTGS |
| | | linker | | |
| 79 | | PT-rich peptide | | GSPTPTPTTPTPTPTTGS |
| | | linker | | |
| 80 | | PT-rich peptide | | GSPTPTPTTGS |
| | | linker | | |
| 81 | | PT-rich peptide | | GSPTPTPTTPTPTPTTPTPTPTGS |
| | | linker | | |
| 82 | | PT-rich peptide | | GSPTPTPTTPTPTPTTPTPTPT |
| | | linker | | |
| 83 | | Consensus GS | | [Gly-Gly-Gly-Gly-Ser]n, wherein n is 1, 2, 3, 4, 5, or 6 |
| | | linker | | |

1. A recombinant protein comprising (i) a binding moiety and (ii) a drug molecule;
wherein said binding moiety reversibly binds to said drug molecule;
wherein said binding moiety, when bound, inhibits a biological activity of said drug molecule; wherein said binding moiety and said drug molecule are connected by a peptide linker; and wherein said peptide linker comprises a protease cleavage site.

2. The recombinant protein of claim 1, wherein said binding moiety comprises an antibody, an alternative scaffold, or a polypeptide.

3. The recombinant protein of claim 1 or 2, wherein said binding moiety comprises an immunoglobulin molecule or a fragment thereof.

4. The recombinant protein of claim 1 or 2, wherein said binding moiety comprises a non-immunoglobulin molecule.

5. The recombinant protein of any of claims 1 to 4, wherein said binding moiety comprises an antigen binding domain that Is derived from a monoclonal antibody, a polyclonal antibody, a recombinant antibody, a chimeric antibody, a human antibody, a humanized antibody, a single-domain antibody, a heavy chain variable domain (VH), a light chain variable domain (VL), or a variable domain (VHH).

6. The recombinant protein of any of claims 1 to 4, wherein said binding moiety comprises an antigen binding domain that is derived from or is related to an adnectin, a monobody, an affibody, an affilin, an affimer, an aptamer, an affitin, an alphabody, an anticalin, a repeat protein domain, an armadillo repeat domain, an atrimer, an avimer, an ankyrin repeat domain, a fynomer, a knottin, a Kunitz domain, or a T cell receptor (TCR).

7. The recombinant protein of any preceding claim, wherein said biological activity of said drug molecule is binding of said drug molecule to a biological target.

8. The recombinant protein of any preceding claim, wherein said biological activity of said drug molecule is an enzymatic activity.

9. The recombinant protein of any preceding claim, wherein cleavage of said peptide linker at said protease cleavage site upon administration of said recombinant protein to a mammal allows release of said drug molecule from the said binding moiety.

10. The recombinant protein of claim 9, wherein said mammal is a human.

11. The recombinant protein of any of claims 9 and 10, wherein said cleavage of said peptide linker occurs in tumor tissue.

12. The recombinant protein of any preceding claim, wherein said protease cleavage site is a site recognized by a protease present in tumor tissue.

13. The recombinant protein of any preceding claim, wherein said binding moiety binds said drug molecule with a dissociation constant (K_{D}) of less than about 1 μM, such as less than about 1 μM, less than about 500 nM, less than about 250 nM, less than about 100 nM or less than about 50 nM.

14. The recombinant protein of any preceding claim, wherein said binding moiety binds said drug molecule with a dissociation constant (K_{D}) of between about 1 μM and about 10 pM, such as of between about 1 μM and about 10 pM, of between about 1 μM and about 20 pM, of between about 1 μM and about 50 pM, or of between about 1 μM and about 100 pM.

15. The recombinant protein of claim 13 or 14, wherein said dissociation constant (K_{D}) is measured in phosphate buffered saline (PBS).

16. The recombinant protein of any preceding claim, wherein said binding moiety comprises a designed ankyrin repeat domain.

17. The recombinant protein of claim 16, wherein said designed ankyrin repeat domain comprises an ankyrin repeat module comprising an amino acid sequence selected from the group consisting of (1) SEQ ID NOs: 45 to 64 and (2) sequences in which up to 9 amino acids in any of SEQ ID NOs: 45 to 64 are substituted by other amino acids.

18. The recombinant protein of any one of claims 16 and 17, wherein said designed ankyrin repeat domain comprises an amino acid sequence selected from the group consisting of (1) SEQ ID NOs: 1 to 12 and (2) sequences that have at least 85% amino acid sequence identity with any of SEQ ID NOs: 1 to 12.

19. The recombinant protein of any preceding claim, wherein said drug molecule comprises an antibody, an alternative scaffold, or a polypeptide.

20. The recombinant protein of any preceding claim, wherein said drug molecule comprises an immunoglobulin molecule or a fragment thereof.

21. The recombinant protein of any preceding claim, wherein said drug molecule comprises a non-immunoglobulin molecule.

22. The recombinant protein of any preceding claim, wherein said drug molecule comprises an antigen binding domain that is derived from a monoclonal antibody, a polyclonal antibody, a recombinant antibody, a chimeric antibody, a human antibody, a humanized antibody, a single-domain antibody, a heavy chain variable domain (VH), a light chain variable domain (VL), or a variable domain (VHH).

23. The recombinant protein of any preceding claim, wherein said drug molecule comprises an antigen binding domain that is derived from or is related to an adnectin, a monobody, an affibody, an affilin, an affimer, an aptamer, an affitin, an alphabody, an anticalin, a repeat protein domain, an armadillo repeat domain, an atrimer, an avimer, an ankyrin repeat domain, a fynomer, a knottin, a Kunitz domain, or a T cell receptor (TCR).

24. The recombinant protein of any preceding claim, wherein said drug molecule has binding specificity for CD3.

25. The recombinant protein of any of any preceding claim, wherein said drug molecule comprises at least one binding domain with binding specificity for a tumor-associated antigen (TAA).

26. The recombinant protein of any preceding claim, wherein said drug molecule comprises a designed ankyrin repeat domain.

27. The recombinant protein of claim 26, wherein said designed ankyrin repeat domain has binding specificity for CD3.

28. The recombinant protein of any of claims 26 and 27, wherein said designed ankyrin repeat domain comprises an amino acid sequence selected from the group consisting of (1) SEQ ID NOs: 13 to 17 and (2) sequences that have at least 85% amino acid sequence identity with any of SEQ ID NOs: 13 to 17.

29. The recombinant protein of any of claims 27 and 28, wherein said designed ankyrin repeat domain binds to CD3 with a dissociation constant (K_{D}) of less than about 100 nM.

30. The recombinant protein of any of claims 1 to 25, wherein said drug molecule comprises an antibody.

31. The recombinant protein of claim 30, wherein said antibody has binding specificity for CD3.

32. The recombinant protein of any preceding claim, wherein said drug molecule is a T-cell engager drug molecule (TCE).

33. The recombinant protein of claim 32, wherein said TCE comprises a binding domain which binds to CD3 and further comprises a binding domain which binds a tumor-associated antigen (TAA).

34. The recombinant protein of any of claims 32 and 33, wherein binding of said binding moiety to said TCE drug molecule inhibits binding of said TCE drug molecule to T cells and/or activation of T cells.

35. The recombinant protein of any one of claims 32 to 34, wherein said TCE is a bispecific or multispecific antibody.

36. The recombinant protein of any one of claims 32 to 34, wherein said TCE is a bispecific or multispecific ankyrin repeat protein.

37. The recombinant protein of any one of claims 33 to 36, wherein said binding domain which binds to CD3 is located on the C-terminal side of said binding domain which binds a tumor-associated antigen (TAA).

38. The recombinant protein of any preceding claim, wherein said binding moiety is an anti-idiotypic binder of said drug molecule.

39. The recombinant protein of claim 38, wherein said binding moiety is an anti-idiotypic binder of said designed ankyrin repeat domain having binding specificity for CD3.

40. The recombinant protein of claim 38, wherein said binding moiety is an anti-idiotypic binder of said antibody having binding specificity for CD3.

41. The recombinant protein of any preceding claim, wherein said binding moiety, said drug molecule and said peptide linker are arranged, from N-terminus to C-terminus, in the following format: drug molecule—peptide linker—binding moiety.

42. The recombinant protein of any of claims 1 to 41, wherein said binding moiety, said binding domain which binds to CD3, said binding domain which binds a tumor-associated antigen (TAA), and said peptide linker are arranged, from N-terminus to C-terminus, in the following format: binding domain which binds a tumor-associated antigen (TAA)—binding domain which binds to CD3—peptide linker—binding moiety.

43. The recombinant protein of any preceding claim, further comprising an agent which extends the serum half-life of the recombinant protein in a mammal.

44. The recombinant protein of claim 43, wherein said agent which extends the serum half-life of the recombinant protein in a mammal has binding specificity for serum albumin.

45. The recombinant protein of claim 44, wherein said agent which extends the serum half-life of the recombinant protein in a mammal comprises a designed ankyrin repeat domain with binding specificity for serum albumin.

46. The recombinant protein of claim 45, wherein said designed ankyrin repeat domain with binding specificity for serum albumin comprises an amino acid sequence selected from the group consisting of (1) SEQ ID NOs: 65 to 67 and (2) sequences that have at least 85% amino acid sequence identity with any of SEQ ID NOs: 65 to 67.

47. The recombinant protein of any of claims 43 to 46, wherein said agent which extends the serum half-life of the recombinant protein in a mammal is located at the same side of said peptide linker as said binding moiety.

48. The recombinant protein of claim 47, wherein said binding moiety and said agent which extends the serum half-life of the recombinant protein in a mammal are both located at the C-terminal side of said peptide linker.

49. The recombinant protein of any of claims 43 to 47, wherein said agent which extends the serum half-life of the recombinant protein in a mammal is located at the C-terminal side of said binding moiety.

50. The recombinant protein of any of claims 43 to 49, wherein said binding moiety, said binding domain which binds to CD3, said binding domain which binds a tumor-associated antigen (TAA), said peptide linker, and said agent which extends the serum half-life of the recombinant protein in a mammal are arranged, from N-terminus to C-terminus, in the following format: binding domain which binds a tumor-associated antigen (TAA)—binding domain which binds to CD3—peptide linker—binding moiety—agent which extends the serum half-life of the recombinant protein in a mammal.

51. A nucleic acid encoding the recombinant protein of any of the preceding claims.

52. A host cell comprising the nucleic acid molecule of claim 51.

53. A method of making the recombinant protein of any one of claims 1 to 50, comprising culturing the host cell of claim 52 under conditions wherein said recombinant protein is expressed.

54. The method of claim 53, wherein said host cell is a prokaryotic host cell.

55. The method of claim 53, wherein said host cell is a eukaryotic host cell.

56. A pharmaceutical composition comprising the recombinant protein of any one of claims 1 to 50 or the nucleic acid of claim 51 and additionally comprising a pharmaceutically acceptable carrier or excipient.

57. The recombinant protein of any one of claims 1 to 50, the nucleic acid of claim 51 or the pharmaceutical composition of claim 56 for use in therapy.

58. The recombinant protein, nucleic acid or pharmaceutical composition for use according to claim 57, for use in treating a proliferative disease, optionally wherein said proliferative disease is cancer.

59. A method of treatment comprising the step of administering to a subject in need thereof the recombinant protein of any one of claims 1 to 50, the nucleic acid of claim 51 or the pharmaceutical composition of claim 56.

60. The method of claim 59, wherein said method is a method of treating a proliferative disease, optionally wherein said proliferative disease is cancer.

61. A method of T cell activation in a subject in need thereof, the method comprising the step of administering to said subject the recombinant protein of any one of claims 1 to 50, the nucleic acid of claim 51 or the pharmaceutical composition of claim 56.

62. A method of controlling release of an active drug molecule in vivo comprising administering the recombinant protein of any one of claims 1 to 50, the nucleic acid of claim 51 or the pharmaceutical composition of claim 56 to a subject in need thereof.

63. The method of any one of claims 59 to 62, wherein said subject is a human.

64. A method of controlling the biological activity of a drug molecule, the method comprising connecting a binding moiety as defined in any one of claims 1 to 6, 13 to 18 and 38 to 40 with a drug molecule as defined in any one of claims 19 to 37 with a peptide linker comprising a protease cleavage site to form a recombinant protein and administering said recombinant protein to a patient in need thereof, wherein said protease cleavage site is recognized by a protease present in tumor tissue.

65. The method of claim 64, wherein said biological activity of said drug molecule is binding of said drug molecule to a biological target.

66. The method of claim 64, wherein said biological activity of said drug molecule is an enzymatic activity.

67. A binding moiety having binding specificity for a drug molecule, wherein said binding moiety, when connected to said drug molecule by a peptide linker, inhibits a biological activity of said drug molecule.

68. The binding moiety of claim 67, wherein binding of said binding moiety to said drug molecule forms a complex that reversibly inhibits a biological activity of said drug molecule.

69. The binding moiety of any of claim 67 or 68, wherein said binding moiety is an anti-idiotypic binder of said drug molecule.

70. The binding moiety of any of claim 67 or 69, wherein said biological activity of said drug molecule Is binding of said drug molecule to a biological target.

71. The binding moiety of any of claims 67 to 69, wherein said biological activity of said drug molecule is an enzymatic activity.

72. The binding moiety of any one of claims 67 to 71, having a binding affinity (K_{D}) to said drug molecule of less than about 1 μM, such as less than about 1 μM, less than about 500 nM, less than about 250 nM, less than about 100 nM or less than about 50 nM.

73. The binding moiety of any one of claims 67 to 72, wherein said binding moiety binds said drug molecule with a dissociation constant (K_{D}) of between about 1 μM and about 10 pM, such as of between about 1 μM and about 10 pM, of between about 1 μM and about 20 pM, of between about 1 μM and about 50 pM, or of between about 1 μM and about 100 pM.

74. The binding moiety of claim 72 or 73, wherein said dissociation constant (K_{D}) is measured in phosphate buffered saline (PBS).

75. The binding moiety of any one of claims 67 to 74, wherein said binding moiety comprises an antibody, an alternative scaffold, or a polypeptide.

76. The binding moiety of any one of claims 67 to 75, wherein said binding moiety comprises an immunoglobulin molecule or a fragment thereof.

77. The binding moiety of any one of claims 67 to 76, wherein said binding moiety comprises a non-immunoglobulin molecule.

78. The binding moiety of any one of claims 67 to 77, wherein said binding moiety comprises an antigen binding domain that is derived from a monoclonal antibody, a polyclonal antibody, a recombinant antibody, a chimeric antibody, a human antibody, a humanized antibody, a single-domain antibody, a heavy chain variable domain (VH), a light chain variable domain (VL), or a variable domain (VHH).

79. The binding moiety of any one of claims 67 to 78, wherein said binding moiety comprises an antigen binding domain that is derived from or is related to an adnectin, a monobody, an affibody, an affilin, an affimer, an aptamer, an affitin, an alphabody, an anticalin, a repeat protein domain, an armadillo repeat domain, an atrimer, an avimer, an ankyrin repeat domain, a fynomer, a knottin, a Kunitz domain, or a T cell receptor (TCR).

80. The binding moiety of any one of claims 67 to 79, wherein said binding moiety comprises a designed ankyrin repeat domain.

81. The binding moiety of claim 80, wherein said designed ankyrin repeat domain comprises an ankyrin repeat module comprising an amino acid sequence selected from the group consisting of (1) SEQ ID NOs: 45 to 64 and (2) sequences in which up to 9 amino acids in any of SEQ ID NOs: 45 to 64 are substituted by other amino acids.

82. The binding moiety of any of claim 80 or 81, wherein said designed ankyrin repeat domain comprises an amino acid sequence selected from the group consisting of (1) SEQ ID NOs: 1 to 12 and (2) sequences that have at least 85% amino acid sequence identity with any of SEQ ID NOs: 1 to 12.

83. A nucleic acid encoding the binding moiety of any of claims 67 to 82.

84. A nucleic acid encoding the designed ankyrin repeat domain of any of claims 80 to 82.

85. A host cell comprising the nucleic add molecule of claim 83 or 84.

86. A method of making the binding moiety according to any one of claims 67 to 82, comprising culturing the host cell of claim 85 under conditions wherein said binding moiety is expressed.

87. The method of claim 86, wherein said host cell is a prokaryotic host cell.

88. The method of claim 86, wherein said host cell is a eukaryotic host cell.

89. A pharmaceutical composition comprising the binding moiety of any one of claims 67 to 82 or the nucleic acid of any one of claims 83 and 84 and additionally comprising a pharmaceutically acceptable carrier or excipient.

90. The binding moiety of any one of claims 67 to 82, the nucleic acid of any one of claims 83 and 84 or the pharmaceutical composition of claim 89 for use in therapy.

91. A method of treatment comprising the step of administering to a subject in need thereof the binding moiety of any one of claims 67 to 82, the nucleic acid of any one of claims 83 and 84 or the pharmaceutical composition of claim 89.


SEQUENCE LISTING (83 sequences)

SEQ ID NO: 1 - protein, 157 aa, Artificial Sequence
  Designed ankyrin repeat domain
DLGKKLLQAARAGQLDEVRELLKAGADVNAKDAKGLTPLHLAAYHGHLEIVEVLLKAGADVNAKDVYGWTPLHIAAASGHLEIVEVLLKAGADVNAKDWLGWTPLHLAASHGHLEIVEVLLKAGADVNAQDKSGKTPADLAARAGHQDIAEVLQKAA

SEQ ID NO: 2 - protein, 157 aa, Artificial Sequence
  Designed ankyrin repeat domain
DLGKKLLQAARAGQLDEVRELLKAGADVNAKDAKGLTPLHLAAYHGHLEIVEVLLKAGADVNAKDVAGWTPLHIAAASGHLEIVEVLLKAGADVNAKDWLGWTPLHLAASHGHLEIVEVLLKAGADVNAQDKSGKTPADLAARAGHQDIAEVLQKAA

SEQ ID NO: 3 - protein, 157 aa, Artificial Sequence
  Designed ankyrin repeat domain
DLGKKLLQAARAGQLDEVRELLKAGADVNAKDAKGLTPLHLAAYHGHLEIVEVLLKAGADVNAKDVYGWTPLHIAAASGHLEIVEVLLKAGADVNAKDALGWTPLHLAASHGHLEIVEVLLKAGADVNAQDKSGKTPADLAARAGHQDIAEVLQKAA

SEQ ID NO: 4 - protein, 157 aa, Artificial Sequence
  Designed ankyrin repeat domain
DLGKKLLQAARAGQLDEVRELLKAGADVNAKDAKGLTPLHLAAYHGHLEIVEVLLKAGADVNAKDVYGWTPLHIAAASGHLEIVEVLLKAGADVNAKDWLGWTPLHLAASAGHLEIVEVLLKAGADVNAQDKSGKTPADLAARAGHQDIAEVLQKAA

SEQ ID NO: 5 - protein, 157 aa, Artificial Sequence
  Designed ankyrin repeat domain
DLGKKLLQAARAGQLDEVRELLKAGADVNAKDAKGLTPLHLAAYHGHLEIVEVLLKAGADVNAKDVYGWTPLHIAAASGHLEIVEVLLKAGADVNAKDWLGITPLHLAASHGHLEIVEVLLKAGADVNAQDKSGKTPADLAARAGHQDIAEVLQKAA

SEQ ID NO: 6 - protein, 157 aa, Artificial Sequence
  Designed ankyrin repeat domain
DLGKKLLQAARAGQLDEVRELLKAGADVNAKDAKGLTPLHLAAYHGHLEIVEVLLKAGADVNAKDVAGWTPLHIAAASGHLEIVEVLLKAGADVNAKDWLGITPLHLAASHGHLEIVEVLLKAGADVNAQDKSGKTPADLAARAGHQDIAEVLQKAA

SEQ ID NO: 7 - protein, 157 aa, Artificial Sequence
  Designed ankyrin repeat domain
DLGKKLLQAARAGQLDEVRELLKAGADVNAKDAKGLTPLHLAAYHGHLEIVEVLLKAGADVNAKDVYGWTPLHIAAASGHLEIVEVLLKAGADVNAKDALGITPLHLAASHGHLEIVEVLLKAGADVNAQDKSGKTPADLAARAGHQDIAEVLQKAA

SEQ ID NO: 8 - protein, 157 aa, Artificial Sequence
  Designed ankyrin repeat domain
DLGKKLLQAARAGQLDEVRELLKAGADVNAKDAKGLTPLHLAAYHGHLEIVEVLLKAGADVNAKDVYGWTPLHIAAASGHLEIVEVLLKAGADVNAKDWLGITPLHLAASAGHLEIVEVLLKAGADVNAQDKSGKTPADLAARAGHQDIAEVLQKAA

SEQ ID NO: 9 - protein, 157 aa, Artificial Sequence
  Designed ankyrin repeat domain
DLGKKLLQAARAGQLDEVRELLKAGADVNAKDAKGLTPLHLAAYHGHLEIVEVLLKAGADVNAKDVYGWTPLHIAAASGHLEIVEVLLKAGADVNAKDWLGATPLHLAASHGHLEIVEVLLKAGADVNAQDKSGKTPADLAARAGHQDIAEVLQKAA

SEQ ID NO: 10 - protein, 157 aa, Artificial Sequence
  Designed ankyrin repeat domain
DLGKKLLQAARAGQLDEVRELLKAGADVNAKDAKGLTPLHLAAYHGHLEIVEVLLKAGADVNAKDVAGWTPLHIAAASGHLEIVEVLLKAGADVNAKDWLGATPLHLAASHGHLEIVEVLLKAGADVNAQDKSGKTPADLAARAGHQDIAEVLQKAA

SEQ ID NO: 11 - protein, 157 aa, Artificial Sequence
  Designed ankyrin repeat domain
DLGKKLLQAARAGQLDEVRELLKAGADVNAKDAKGLTPLHLAAYHGHLEIVEVLLKAGADVNAKDVYGWTPLHIAAASGHLEIVEVLLKAGADVNAKDALGATPLHLAASHGHLEIVEVLLKAGADVNAQDKSGKTPADLAARAGHQDIAEVLQKAA

SEQ ID NO: 12 - protein, 157 aa, Artificial Sequence
  Designed ankyrin repeat domain
DLGKKLLQAARAGQLDEVRELLKAGADVNAKDAKGLTPLHLAAYHGHLEIVEVLLKAGADVNAKDVYGWTPLHIAAASGHLEIVEVLLKAGADVNAKDWLGATPLHLAASAGHLEIVEVLLKAGADVNAQDKSGKTPADLAARAGHQDIAEVLQKAA

SEQ ID NO: 13 - protein, 124 aa, Artificial Sequence
  CD3-specific binding domain
DLGQKLLEAAWAGQDDEVRILLAAGADVNAKNSRGWTPLHTAAQTGHLEIFEVLLKAGADVNAKNDKRVTPLHLAAALGHLEIVEVLLKAGADVNARDSWGTTPADLAAKYGHGDIAEVLQKAA

SEQ ID NO: 14 - protein, 124 aa, Artificial Sequence
  CD3-specific binding domain
DLGQKLLEAAWAGQDDEVRELLKAGADVNAKDSQGWTPLHTAAQTGHLEIFEVLLKAGADVNAKDDKGVTPLHLAAALGHLEIVEVLLKAGADVNAQDSWGTTPADLAAKYGHEDIAEVLQKAA

SEQ ID NO: 15 - protein, 124 aa, Artificial Sequence
  CD3-specific binding domain
DLGQKLLEAAWAGQDDEVRELLKAGADVNAKNSRGWTPLHTAAQTGHLEIFEVLLKAGADVNAKDDKGVTPLHLAAALGHLEIVEVLLKAGADVNAQDSWGTTPADLAAKYGHEDIAEVLQKAA

SEQ ID NO: 16 - protein, 124 aa, Artificial Sequence
  CD3-specific binding domain
DLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAA

SEQ ID NO: 17 - protein, 124 aa, Artificial Sequence
  CD3-specific binding domain
DLGQKLLEAAWAGQLDEVRILLKAGADVNAKNSRGWTPLHTAAQTGHLEIFEVLLKAGADVNAKTNKRVTPLHLAAALGHLEIVEVLLKAGADVNARDTWGTTPADLAAKYGHRDIAEVLQKAA

SEQ ID NO: 18 - protein, 26 aa, Artificial Sequence
  Cleavable linker sequence #1
GSGSGGSGGLSGRSDNHGGSGGSGGS

SEQ ID NO: 19 - protein, 21 aa, Artificial Sequence
  Cleavable linker sequence #2
GSGGGGPQASTGRSGGGGGGS

SEQ ID NO: 20 - protein, 23 aa, Artificial Sequence
  Cleavable linker sequence #3
GSGSPQGIWGPLSGRSDNHGSGS

SEQ ID NO: 21 - DNA, 471 bp, Artificial Sequence
  Nucleic acid encoding a designed ankyrin repeat domain
gacttaggaaagaaattgctgcaagccgcacgcgccggtcaacttgatgaggtgcgcgaattattgaaggcaggtgcagacgtgaacgctaaagacgctaagggacttactcctttacacttagcggcctatcatggtcatttggaaattgtggaggtcctgttgaaggctggtgccgacgtgaacgccaaagatgtttacggttggaccccattacacattgctgccgcctcgggacatctggaaattgttgaggttctgcttaaagctggcgcagacgttaatgccaaggactggttggggtggacgcccttacacctggccgcgtcacatggacatttagagattgtagaagtcctgttaaaggcgggcgcggacgttaatgcccaagacaaaagtggcaaaacaccagcggatctggccgctcgtgctggacaccaggacattgctgaagtgctgcagaaggcagcg

SEQ ID NO: 22 - DNA, 471 bp, Artificial Sequence
  Nucleic acid encoding a designed ankyrin repeat domain
gacttaggaaagaaattgctgcaagccgcacgcgccggtcaacttgatgaggtgcgcgaattattgaaggcaggtgcagacgtgaacgctaaagacgctaagggacttactcctttacacttagcggcctatcatggtcatttggaaattgtggaggtcctgttgaaggctggcgccgacgtgaacgccaaagatgttgcaggttggaccccattacacattgctgccgcctcgggacatctggaaattgttgaggttctgcttaaagctggcgcagacgttaatgccaaggactggttggggtggacgcccttacacctggccgcgtcacatggacatttagagattgtagaagtcctgttaaaggcgggcgcggacgttaatgcccaagacaaaagtggcaaaacaccagcggatctggccgctcgtgctggacaccaggacattgctgaagtgctgcagaaggcagcg

SEQ ID NO: 23 - DNA, 471 bp, Artificial Sequence
  Nucleic acid encoding a designed ankyrin repeat domain
gacttaggaaagaaattgctgcaagccgcacgcgccggtcaacttgatgaggtgcgcgaattattgaaggcaggtgcagacgtgaacgctaaagacgctaagggacttactcctttacacttagcggcctatcatggtcatttggaaattgtggaggtcctgttgaaggctggcgccgacgtgaacgccaaagatgtttacggttggaccccattacacattgctgccgcctcgggacatctggaaattgttgaggttctgcttaaagctggcgcagacgttaatgccaaggacgcattggggtggacgcccttacacctggccgcgtcacatggacatttagagattgtagaagtcctgttaaaggcgggcgcggacgttaatgcccaagacaaaagtggcaaaacaccagcggatctggccgctcgtgctggacaccaggacattgctgaagtgctgcagaaggcagcg

SEQ ID NO: 24 - DNA, 471 bp, Artificial Sequence
  Nucleic acid encoding a designed ankyrin repeat domain
gacttaggaaagaaattgctgcaagccgcacgcgccggtcaacttgatgaggtgcgcgaattattgaaggcaggtgcagacgtgaacgctaaagacgctaagggacttactcctttacacttagcggcctatcatggtcatttggaaattgtggaggtcctgttgaaggctggtgccgacgtgaacgccaaagatgtttacggttggaccccattacacattgctgccgcctcgggacatctggaaattgttgaggttctgcttaaagctggcgcagacgttaatgccaaggactggttggggtggacgcccttacacctggccgcgtcagcaggacatttagagattgtagaagtcctgttaaaggcgggcgcggacgttaatgcccaagacaaaagtggcaaaacaccagcggatctggccgctcgtgctggacaccaggacattgctgaagtgctgcagaaggcagcg

SEQ ID NO: 25 - protein, 12 aa, Artificial Sequence
  His-tag
MRGSHHHHHHGS

SEQ ID NO: 26 - protein, 8 aa, Artificial Sequence
  His-tag
GSHHHHHH

SEQ ID NO: 27 - protein, 127 aa, Artificial Sequence
  EGFR binding domain used in the CD3-PDD constructs
MGSDLGYKLLRAAFHGQDDEVRILLAAGADVNAKDLIGQTPLHNAAWVGHLEIVEVLLKAGADVNAKDYYGNTPLHLAAHDGHLEIVEVLLKAGADVNAQDTWGETPADLATNAGHEDIAEVLQKAA

SEQ ID NO: 28 - protein, 24 aa, Artificial Sequence
  Non-cleavable linker
  VARIANT 1,2: wherein Xaa Xaa represents GS or SG
XXPTPTPTTPTPTPTTPTPTPTGS

SEQ ID NO: 29 - protein, 283 aa, Artificial Sequence
  Full sequence of active TCE
MGSDLGYKLLRAAFHGQDDEVRILLAAGADVNAKDLIGQTPLHNAAWVGHLEIVEVLLKAGADVNAKDYYGNTPLHLAAHDGHLEIVEVLLKAGADVNAQDTWGETPADLATNAGHEDIAEVLQKAAGSPTPTPTTPTPTPTTPTPTPTGSDLGQKLLEAAWAGQDDEVRILLAAGADVNAKNSRGWTPLHTAAQTGHLEIFEVLLKAGADVNAKNDKRVTPLHLAAALGHLEIVEVLLKAGADVNARDSWGTTPADLAAKYGHGDIAEVLQKAAGSHHHHHH

SEQ ID NO: 30 - protein, 283 aa, Artificial Sequence
  Full sequence of active TCE
MGSDLGYKLLRAAFHGQDDEVRILLAAGADVNAKDLIGQTPLHNAAWVGHLEIVEVLLKAGADVNAKDYYGNTPLHLAAHDGHLEIVEVLLKAGADVNAQDTWGETPADLATNAGHEDIAEVLQKAAGSPTPTPTTPTPTPTTPTPTPTGSDLGQKLLEAAWAGQDDEVRELLKAGADVNAKNSRGWTPLHTAAQTGHLEIFEVLLKAGADVNAKDDKGVTPLHLAAALGHLEIVEVLLKAGADVNAQDSWGTTPADLAAKYGHEDIAEVLQKAAGSHHHHHH

SEQ ID NO: 31 - protein, 283 aa, Artificial Sequence
  Full sequence of active TCE
MGSDLGYKLLRAAFHGQDDEVRILLAAGADVNAKDLIGQTPLHNAAWVGHLEIVEVLLKAGADVNAKDYYGNTPLHLAAHDGHLEIVEVLLKAGADVNAQDTWGETPADLATNAGHEDIAEVLQKAAGSPTPTPTTPTPTPTTPTPTPTGSDLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAAGSHHHHHH

SEQ ID NO: 32 - protein, 464 aa, Artificial Sequence
  Full sequence of CD3-PDD NCL
MGSDLGYKLLRAAFHGQDDEVRILLAAGADVNAKDLIGQTPLHNAAWVGHLEIVEVLLKAGADVNAKDYYGNTPLHLAAHDGHLEIVEVLLKAGADVNAQDTWGETPADLATNAGHEDIAEVLQKAAGSPTPTPTTPTPTPTTPTPTPTGSDLGQKLLEAAWAGQDDEVRILLAAGADVNAKNSRGWTPLHTAAQTGHLEIFEVLLKAGADVNAKNDKRVTPLHLAAALGHLEIVEVLLKAGADVNARDSWGTTPADLAAKYGHGDIAEVLQKAASGPTPTPTTPTPTPTTPTPTPTGSDLGKKLLQAARAGQLDEVRELLKAGADVNAKDAKGLTPLHLAAYHGHLEIVEVLLKAGADVNAKDVYGWTPLHIAAASGHLEIVEVLLKAGADVNAKDWLGWTPLHLAASHGHLEIVEVLLKAGADVNAQDKSGKTPADLAARAGHQDIAEVLQKAAGSHHHHHH

SEQ ID NO: 33 - protein, 464 aa, Artificial Sequence
  Full sequence of CD3-PDD NCL
MGSDLGYKLLRAAFHGQDDEVRILLAAGADVNAKDLIGQTPLHNAAWVGHLEIVEVLLKAGADVNAKDYYGNTPLHLAAHDGHLEIVEVLLKAGADVNAQDTWGETPADLATNAGHEDIAEVLQKAAGSPTPTPTTPTPTPTTPTPTPTGSDLGQKLLEAAWAGQDDEVRILLAAGADVNAKNSRGWTPLHTAAQTGHLEIFEVLLKAGADVNAKNDKRVTPLHLAAALGHLEIVEVLLKAGADVNARDSWGTTPADLAAKYGHGDIAEVLQKAASGPTPTPTTPTPTPTTPTPTPTGSDLGKKLLQAARAGQLDEVRELLKAGADVNAKDAKGLTPLHLAAYHGHLEIVEVLLKAGADVNAKDVYGWTPLHIAAASGHLEIVEVLLKAGADVNAKDALGWTPLHLAASHGHLEIVEVLLKAGADVNAQDKSGKTPADLAARAGHQDIAEVLQKAAGSHHHHHH

SEQ ID NO: 34 - protein, 612 aa, Artificial Sequence
  Full sequence of half-life extended CD3-PDD NCL
MGSDLGYKLLRAAFHGQDDEVRILLAAGADVNAKDLIGQTPLHNAAWVGHLEIVEVLLKAGADVNAKDYYGNTPLHLAAHDGHLEIVEVLLKAGADVNAQDTWGETPADLATNAGHEDIAEVLQKAAGSPTPTPTTPTPTPTTPTPTPTGSDLGQKLLEAAWAGQDDEVRILLAAGADVNAKNSRGWTPLHTAAQTGHLEIFEVLLKAGADVNAKNDKRVTPLHLAAALGHLEIVEVLLKAGADVNARDSWGTTPADLAAKYGHGDIAEVLQKAASGPTPTPTTPTPTPTTPTPTPTGSDLGKKLLQAARAGQLDEVRELLKAGADVNAKDAKGLTPLHLAAYHGHLEIVEVLLKAGADVNAKDVYGWTPLHIAAASGHLEIVEVLLKAGADVNAKDWLGWTPLHLAASHGHLEIVEVLLKAGADVNAQDKSGKTPADLAARAGHQDIAEVLQKAAGSPTPTPTTPTPTPTTPTPTPTGSDLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAAGSHHHHHH

SEQ ID NO: 35 - protein, 612 aa, Artificial Sequence
  Full sequence of half-life extended CD3-PDD NCL
MGSDLGYKLLRAAFHGQDDEVRILLAAGADVNAKDLIGQTPLHNAAWVGHLEIVEVLLKAGADVNAKDYYGNTPLHLAAHDGHLEIVEVLLKAGADVNAQDTWGETPADLATNAGHEDIAEVLQKAAGSPTPTPTTPTPTPTTPTPTPTGSDLGQKLLEAAWAGQDDEVRILLAAGADVNAKNSRGWTPLHTAAQTGHLEIFEVLLKAGADVNAKNDKRVTPLHLAAALGHLEIVEVLLKAGADVNARDSWGTTPADLAAKYGHGDIAEVLQKAASGPTPTPTTPTPTPTTPTPTPTGSDLGKKLLQAARAGQLDEVRELLKAGADVNAKDAKGLTPLHLAAYHGHLEIVEVLLKAGADVNAKDVYGWTPLHIAAASGHLEIVEVLLKAGADVNAKDALGWTPLHLAASHGHLEIVEVLLKAGADVNAQDKSGKTPADLAARAGHQDIAEVLQKAAGSPTPTPTTPTPTPTTPTPTPTGSDLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAAGSHHHHHH

SEQ ID NO: 36 - protein, 464 aa, Artificial Sequence
  Full sequence of CD3-PDD NCL
MGSDLGYKLLRAAFHGQDDEVRILLAAGADVNAKDLIGQTPLHNAAWVGHLEIVEVLLKAGADVNAKDYYGNTPLHLAAHDGHLEIVEVLLKAGADVNAQDTWGETPADLATNAGHEDIAEVLQKAAGSPTPTPTTPTPTPTTPTPTPTGSDLGQKLLEAAWAGQDDEVRELLKAGADVNAKNSRGWTPLHTAAQTGHLEIFEVLLKAGADVNAKDDKGVTPLHLAAALGHLEIVEVLLKAGADVNAQDSWGTTPADLAAKYGHEDIAEVLQKAASGPTPTPTTPTPTPTTPTPTPTGSDLGKKLLQAARAGQLDEVRELLKAGADVNAKDAKGLTPLHLAAYHGHLEIVEVLLKAGADVNAKDVYGWTPLHIAAASGHLEIVEVLLKAGADVNAKDALGWTPLHLAASHGHLEIVEVLLKAGADVNAQDKSGKTPADLAARAGHQDIAEVLQKAAGSHHHHHH

SEQ ID NO: 37 - protein, 464 aa, Artificial Sequence
  Full sequence of CD3-PDD NCL
MGSDLGYKLLRAAFHGQDDEVRILLAAGADVNAKDLIGQTPLHNAAWVGHLEIVEVLLKAGADVNAKDYYGNTPLHLAAHDGHLEIVEVLLKAGADVNAQDTWGETPADLATNAGHEDIAEVLQKAAGSPTPTPTTPTPTPTTPTPTPTGSDLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAASGPTPTPTTPTPTPTTPTPTPTGSDLGKKLLQAARAGQLDEVRELLKAGADVNAKDAKGLTPLHLAAYHGHLEIVEVLLKAGADVNAKDVYGWTPLHIAAASGHLEIVEVLLKAGADVNAKDALGWTPLHLAASHGHLEIVEVLLKAGADVNAQDKSGKTPADLAARAGHQDIAEVLQKAAGSHHHHHH

SEQ ID NO: 38 - protein, 464 aa, Artificial Sequence
  Full sequence of CD3-PDD NCL
MGSDLGYKLLRAAFHGQDDEVRILLAAGADVNAKDLIGQTPLHNAAWVGHLEIVEVLLKAGADVNAKDYYGNTPLHLAAHDGHLEIVEVLLKAGADVNAQDTWGETPADLATNAGHEDIAEVLQKAAGSPTPTPTTPTPTPTTPTPTPTGSDLGQKLLEAAWAGQDDEVRELLKAGADVNAKNSRGWTPLHTAAQTGHLEIFEVLLKAGADVNAKDDKGVTPLHLAAALGHLEIVEVLLKAGADVNAQDSWGTTPADLAAKYGHEDIAEVLQKAASGPTPTPTTPTPTPTTPTPTPTGSDLGKKLLQAARAGQLDEVRELLKAGADVNAKDAKGLTPLHLAAYHGHLEIVEVLLKAGADVNAKDVYGWTPLHIAAASGHLEIVEVLLKAGADVNAKDWLGWTPLHLAASAGHLEIVEVLLKAGADVNAQDKSGKTPADLAARAGHQDIAEVLQKAAGSHHHHHH

SEQ ID NO: 39 - protein, 464 aa, Artificial Sequence
  Full sequence of CD3-PDD NCL
MGSDLGYKLLRAAFHGQDDEVRILLAAGADVNAKDLIGQTPLHNAAWVGHLEIVEVLLKAGADVNAKDYYGNTPLHLAAHDGHLEIVEVLLKAGADVNAQDTWGETPADLATNAGHEDIAEVLQKAAGSPTPTPTTPTPTPTTPTPTPTGSDLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAASGPTPTPTTPTPTPTTPTPTPTGSDLGKKLLQAARAGQLDEVRELLKAGADVNAKDAKGLTPLHLAAYHGHLEIVEVLLKAGADVNAKDVYGWTPLHIAAASGHLEIVEVLLKAGADVNAKDWLGWTPLHLAASAGHLEIVEVLLKAGADVNAQDKSGKTPADLAARAGHQDIAEVLQKAAGSHHHHHH

SEQ ID NO: 40 - protein, 466 aa, Artificial Sequence
  Full sequence of CD3-PDD CL with cleavable linker #1
MGSDLGYKLLRAAFHGQDDEVRILLAAGADVNAKDLIGQTPLHNAAWVGHLEIVEVLLKAGADVNAKDYYGNTPLHLAAHDGHLEIVEVLLKAGADVNAQDTWGETPADLATNAGHEDIAEVLQKAAGSPTPTPTTPTPTPTTPTPTPTGSDLGQKLLEAAWAGQDDEVRILLAAGADVNAKNSRGWTPLHTAAQTGHLEIFEVLLKAGADVNAKNDKRVTPLHLAAALGHLEIVEVLLKAGADVNARDSWGTTPADLAAKYGHGDIAEVLQKAAGSGSGGSGGLSGRSDNHGGSGGSGGSDLGKKLLQAARAGQLDEVRELLKAGADVNAKDAKGLTPLHLAAYHGHLEIVEVLLKAGADVNAKDVYGWTPLHIAAASGHLEIVEVLLKAGADVNAKDWLGWTPLHLAASHGHLEIVEVLLKAGADVNAQDKSGKTPADLAARAGHQDIAEVLQKAAGSHHHHHH

SEQ ID NO: 41 - protein, 461 aa, Artificial Sequence
  Full sequence of CD3-PDD CL with cleavable linker #2
MGSDLGYKLLRAAFHGQDDEVRILLAAGADVNAKDLIGQTPLHNAAWVGHLEIVEVLLKAGADVNAKDYYGNTPLHLAAHDGHLEIVEVLLKAGADVNAQDTWGETPADLATNAGHEDIAEVLQKAAGSPTPTPTTPTPTPTTPTPTPTGSDLGQKLLEAAWAGQDDEVRILLAAGADVNAKNSRGWTPLHTAAQTGHLEIFEVLLKAGADVNAKNDKRVTPLHLAAALGHLEIVEVLLKAGADVNARDSWGTTPADLAAKYGHGDIAEVLQKAAGSGGGGPQASTGRSGGGGGGSDLGKKLLQAARAGQLDEVRELLKAGADVNAKDAKGLTPLHLAAYHGHLEIVEVLLKAGADVNAKDVYGWTPLHIAAASGHLEIVEVLLKAGADVNAKDALGWTPLHLAASHGHLEIVEVLLKAGADVNAQDKSGKTPADLAARAGHQDIAEVLQKAAGSHHHHHH

SEQ ID NO: 42 - protein, 463 aa, Artificial Sequence
  Full sequence of CD3-PDD CL with cleavable linker #3
MGSDLGYKLLRAAFHGQDDEVRILLAAGADVNAKDLIGQTPLHNAAWVGHLEIVEVLLKAGADVNAKDYYGNTPLHLAAHDGHLEIVEVLLKAGADVNAQDTWGETPADLATNAGHEDIAEVLQKAAGSPTPTPTTPTPTPTTPTPTPTGSDLGQKLLEAAWAGQDDEVRILLAAGADVNAKNSRGWTPLHTAAQTGHLEIFEVLLKAGADVNAKNDKRVTPLHLAAALGHLEIVEVLLKAGADVNARDSWGTTPADLAAKYGHGDIAEVLQKAAGSGSPQGIWGPLSGRSDNHGSGSDLGKKLLQAARAGQLDEVRELLKAGADVNAKDAKGLTPLHLAAYHGHLEIVEVLLKAGADVNAKDVYGWTPLHIAAASGHLEIVEVLLKAGADVNAKDALGWTPLHLAASHGHLEIVEVLLKAGADVNAQDKSGKTPADLAARAGHQDIAEVLQKAAGSHHHHHH

SEQ ID NO: 43 - protein, 461 aa, Artificial Sequence
  Full sequence of CD3-PDD CL with cleavable linker #2
MGSDLGYKLLRAAFHGQDDEVRILLAAGADVNAKDLIGQTPLHNAAWVGHLEIVEVLLKAGADVNAKDYYGNTPLHLAAHDGHLEIVEVLLKAGADVNAQDTWGETPADLATNAGHEDIAEVLQKAAGSPTPTPTTPTPTPTTPTPTPTGSDLGQKLLEAAWAGQDDEVRELLKAGADVNAKNSRGWTPLHTAAQTGHLEIFEVLLKAGADVNAKDDKGVTPLHLAAALGHLEIVEVLLKAGADVNAQDSWGTTPADLAAKYGHEDIAEVLQKAASGGGGGPQASTGRSGGGGGGSDLGKKLLQAARAGQLDEVRELLKAGADVNAKDAKGLTPLHLAAYHGHLEIVEVLLKAGADVNAKDVYGWTPLHIAAASGHLEIVEVLLKAGADVNAKDWLGWTPLHLAASAGHLEIVEVLLKAGADVNAQDKSGKTPADLAARAGHQDIAEVLQKAAGSHHHHHH

SEQ ID NO: 44 - protein, 461 aa, Artificial Sequence
  Full sequence of CD3-PDD CL with cleavable linker #2
MGSDLGYKLLRAAFHGQDDEVRILLAAGADVNAKDLIGQTPLHNAAWVGHLEIVEVLLKAGADVNAKDYYGNTPLHLAAHDGHLEIVEVLLKAGADVNAQDTWGETPADLATNAGHEDIAEVLQKAAGSPTPTPTTPTPTPTTPTPTPTGSDLGQKLLEAAWAGQDDEVRELLKAGADVNAKNSRGWTPLHTAAQTGHLEIFEVLLKAGADVNAKNDKRVTPLHLAAALGHLEIVEVLLKAGADVNARDSWGTTPADLAAKYGHQDIAEVLQKAASGGGGGPQASTGRSGGGGGGSDLGKKLLQAARAGQLDEVRELLKAGADVNAKDAKGLTPLHLAAYHGHLEIVEVLLKAGADVNAKDVYGWTPLHIAAASGHLEIVEVLLKAGADVNAKDWLGWTPLHLAASAGHLEIVEVLLKAGADVNAQDKSGKTPADLAARAGHQDIAEVLQKAAGSHHHHHH

SEQ ID NO: 45 - protein, 33 aa, Artificial Sequence
  Ankyrin repeat module
KDAKGLTPLHLAAYHGHLEIVEVLLKAGADVNA

SEQ ID NO: 46 - protein, 33 aa, Artificial Sequence
  Ankyrin repeat module
KDVYGWTPLHIAAASGHLEIVEVLLKAGADVNA

SEQ ID NO: 47 - protein, 33 aa, Artificial Sequence
  Ankyrin repeat module
KDWLGWTPLHLAASHGHLEIVEVLLKAGADVNA

SEQ ID NO: 48 - protein, 33 aa, Artificial Sequence; identical to SEQ ID NO: 45
  Ankyrin repeat module

SEQ ID NO: 49 - protein, 33 aa, Artificial Sequence
  Ankyrin repeat module
KDVAGWTPLHIAAASGHLEIVEVLLKAGADVNA

SEQ ID NO: 50 - protein, 33 aa, Artificial Sequence; identical to SEQ ID NO: 47
  Ankyrin repeat module

SEQ ID NO: 51 - protein, 33 aa, Artificial Sequence; identical to SEQ ID NO: 45
  Ankyrin repeat module

SEQ ID NO: 52 - protein, 33 aa, Artificial Sequence; identical to SEQ ID NO: 46
  Ankyrin repeat module

SEQ ID NO: 53 - protein, 33 aa, Artificial Sequence
  Ankyrin repeat module
KDALGWTPLHLAASHGHLEIVEVLLKAGADVNA

SEQ ID NO: 54 - protein, 33 aa, Artificial Sequence; identical to SEQ ID NO: 45
  Ankyrin repeat module

SEQ ID NO: 55 - protein, 33 aa, Artificial Sequence; identical to SEQ ID NO: 46
  Ankyrin repeat module

SEQ ID NO: 56 - protein, 33 aa, Artificial Sequence
  Ankyrin repeat module
KDWLGWTPLHLAASAGHLEIVEVLLKAGADVNA

SEQ ID NO: 57 - protein, 33 aa, Artificial Sequence
  Ankyrin repeat module
KDWLGITPLHLAASHGHLEIVEVLLKAGADVNA

SEQ ID NO: 58 - protein, 33 aa, Artificial Sequence; identical to SEQ ID NO: 57
  Ankyrin repeat module

SEQ ID NO: 59 - protein, 33 aa, Artificial Sequence
  Ankyrin repeat module
KDALGITPLHLAASHGHLEIVEVLLKAGADVNA

SEQ ID NO: 60 - protein, 33 aa, Artificial Sequence
  Ankyrin repeat module
KDWLGITPLHLAASAGHLEIVEVLLKAGADVNA

SEQ ID NO: 61 - protein, 33 aa, Artificial Sequence
  Ankyrin repeat module
KDWLGATPLHLAASHGHLEIVEVLLKAGADVNA

SEQ ID NO: 62 - protein, 33 aa, Artificial Sequence; identical to SEQ ID NO: 61
  Ankyrin repeat module

SEQ ID NO: 63 - protein, 33 aa, Artificial Sequence
  Ankyrin repeat module
KDALGATPLHLAASHGHLEIVEVLLKAGADVNA

SEQ ID NO: 64 - protein, 33 aa, Artificial Sequence
  Ankyrin repeat module
KDWLGATPLHLAASAGHLEIVEVLLKAGADVNA

SEQ ID NO: 65 - protein, 124 aa, Artificial Sequence
  HSA-specific binding domain
DLGKKLLEAARAGQDDEVRELLKAGADVNAKDYFSHTPLHLAARNGHLKIVEVLLKAGADVNAKDFAGKTPLHLAANEGHLEIVEVLLKAGADVNAQDIFGKTPADIAADAGHEDIAEVLQKAA

SEQ ID NO: 66 - protein, 124 aa, Artificial Sequence
  HSA-specific binding domain
DLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAA

SEQ ID NO: 67 - protein, 124 aa, Artificial Sequence
  HSA-specific binding domain
DLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAA

SEQ ID NO: 68 - protein, 33 aa, Artificial Sequence
  Ankyrin repeat module
  VARIANT 1,3,4,6,13,14,15,17,18,19,22,26,27: wherein Xaa denotes any amino acid (preferably not cysteine, glycine, or proline)
XDXXGXTPLHLAXXXGXXXLVXVLLXXGADVNA

SEQ ID NO: 69 - protein, 33 aa, Artificial Sequence
  Ankyrin repeat module
  VARIANT 1,3,4,6,14,15,27: wherein Xaa in position 1, 3, 4, 6, 14, 15 denotes any amino acid (preferably not cysteine, glycine, or proline), and Xaa in position 27 is selected from the group consisting of asparagine, histidine, or tyrosine
XDXXGXTPLHLAAXXGHLEIVEVLLKXGADVNA

SEQ ID NO: 70 - protein, 32 aa, Artificial Sequence
  N-cap
GSDLGKKLLQAARAGQLDEVRELLKAGADVNA

SEQ ID NO: 71 - protein, 32 aa, Artificial Sequence
  N-cap
GSDLGKKLLQAARAGQLDEVRILLKAGADVNA

SEQ ID NO: 72 - protein, 32 aa, Artificial Sequence
  N-cap
GSDLGKKLLQAARAGQLDEVRILLAAGADVNA

SEQ ID NO: 73 - protein, 32 aa, Artificial Sequence
  N-Cap (randomized)
  VARIANT 6,13,14: wherein Xaa denotes any amino acid wherein Xaa denotes any amino acid
GSDLGXKLLQAAXXGQLDEVRILLKAGADVNA

SEQ ID NO: 74 - protein, 28 aa, Artificial Sequence
  C-cap
QDKFGKTPADIAADNGHEDIAEVLQKLN

SEQ ID NO: 75 - protein, 28 aa, Artificial Sequence
  C-cap
QDKSGKTPADLAARAGHQDIAEVLQKAA

SEQ ID NO: 76 - protein, 28 aa, Artificial Sequence
  C-cap
QDSSGFTPADLAALVGHEDIAEVLQKAA

SEQ ID NO: 77 - protein, 28 aa, Artificial Sequence
  C-cap (randomized)
  VARIANT 3,4,6,15: wherein Xaa denotes any amino acid
QDXXGXTPADLAARXGHQDIAEVLQKAA

SEQ ID NO: 78 - protein, 38 aa, Artificial Sequence
  PT-rich peptide linker
GSPTPTPTTPTPTPTTPTPTPTTPTPTPTTPTPTPTGS

SEQ ID NO: 79 - protein, 18 aa, Artificial Sequence
  PT-rich peptide linker
GSPTPTPTTPTPTPTTGS

SEQ ID NO: 80 - protein, 11 aa, Artificial Sequence
  PT-rich peptide linker
GSPTPTPTTGS

SEQ ID NO: 81 - protein, 24 aa, Artificial Sequence
  PT-rich peptide linker
GSPTPTPTTPTPTPTTPTPTPTGS

SEQ ID NO: 82 - protein, 22 aa, Artificial Sequence
  PT-rich peptide linker
GSPTPTPTTPTPTPTTPTPTPT

SEQ ID NO: 83 - protein, 5 aa, Artificial Sequence
  Consensus GS linker
  VARIANT 1..5: [Gly-Gly-Gly-Gly-Ser]n, wherein n is 1, 2, 3, 4, 5, or 6
GGGGS